(19) United States
(12) Patent Application Publication
(10) Pub. No.: US 2026/0016486 A1
(43) Pub. Date: Jan. 15, 2026

(54) COMPOSITIONS, DEVICES, AND METHODS OF CROHN'S DISEASE SENSITIVITY TESTING

(71) Applicant: Biomerica, Inc., Irvine, CA (US)

(72) Inventors: Zackary Irani-Cohen, Irvine, CA (US); Elisabeth Laderman, Irvine, CA (US)

(21) Appl. No.: 19/211,870

(22) Filed: May 19, 2025

Related U.S. Application Data
(63) Continuation of application No. 16/171,154, filed on Oct. 25, 2018, now abandoned.
Application No. 16/171,154 is a continuation of application No. PCT/US2017/028666, filed on Apr. 20, 2017.
(60) Provisional application No. 62/327,917, filed on Apr. 26, 2016.

(51) Int. Cl. G01N 33/68; G01N 33/543; G01N 33/544; G01N 33/564

(52) U.S. Cl. CPC G01N 33/6854; G01N 33/543; G01N 33/544; G01N 33/564; G01N 2800/065

(57) ABSTRACT

Contemplated test kits and methods for food sensitivity are based on rational-based selection of food preparations with established discriminatory p-value. Particularly preferred kits include those with a minimum number of food preparations that have an average discriminatory p-value of ≤0.07 as determined by their raw p-value or an average discriminatory p-value of ≤0.10 as determined by FDR multiplicity adjusted p-value. In further contemplated aspects, compositions and methods for food sensitivity are also stratified by gender to further enhance predictive value.

### RELATED APPLICATIONS

This application is a continuation of U.S. patent application Ser. No. 16/171,154, filed Oct. 25, 2018, which is a continuation of International Application No. PCT/US2017/028666, filed Apr. 20, 2017, which claims priority to U.S. Provisional Patent Application No. 62/327,917, filed Apr. 26, 2016. Each of the foregoing applications is incorporated herein by reference in their entirety.

### FIELD OF THE INVENTION

The field of the invention is sensitivity testing for food intolerance, and especially as it relates to testing and possible elimination of selected food items as trigger foods for patients diagnosed with or suspected to have Crohn's Disease.

### BACKGROUND

The background description includes information that may be useful in understanding the present invention. It is not an admission that any of the information provided herein is prior art or relevant to the presently claimed invention, or that any publication specifically or implicitly referenced is prior art.

Food sensitivity, especially as it relates to Crohn's Disease (a type of inflammatory bowel disease), often presents with diarrhea, rectal bleeding, abdominal cramps and pain, and/or change in bowel habits and underlying causes of Crohn's disease are not well understood in the medical community. Most typically, Crohn's Disease is diagnosed by endoscopic and radiological tests, along with blood tests to identify inflammatory conditions. Unfortunately, treatment of Crohn's disease is often less than effective and may present new difficulties due to immune suppressive or modulatory effects. Elimination of other one or more food items has also shown promise in at least reducing incidence and/or severity of the symptoms. However, Crohn's disease is often quite diverse with respect to dietary items triggering symptoms, and no standardized test to help identify trigger food items with a reasonable degree of certainty is known, leaving such patients often to trial-and-error.

While there are some commercially available tests and labs to help identify trigger foods, the quality of the test results from these labs is generally poor as is reported by a consumer advocacy group (e.g., http://www.which.co.uk/news/2008/08/food-allergy-tests-could-risk-your-health-154711/). Most notably, problems associated with these tests and labs were high false positive rates, high false negative rates, high intra-patient variability, and inter-laboratory variability, rendering such tests nearly useless. Similarly, further inconclusive and highly variable test results were also reported elsewhere (Alternative Medicine Review, Vol. 9, No. 2, 2004: pp 198-207), and the authors concluded that this may be due to food reactions and food sensitivities occurring via a number of different mechanisms. For example, not all Crohn's Disease patients show positive response to food A, and not all Crohn's Disease patients show negative response to food B. Thus, even if a Crohn's Disease patient shows positive response to food A, removal of food A from the patient's diet may not relieve the patient's Crohn's Disease symptoms. In other words, it is not well determined whether food samples used in the currently available tests are properly selected based on the high probabilities to correlate sensitivities to those food samples to Crohn's Disease.

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Thus, even though various tests for food sensitivities are known in the art, all or almost all of them suffer from one or more disadvantages. Therefore, there is still a need for improved compositions, devices, and methods of food sensitivity testing, especially for identification and possible elimination of trigger foods for patients identified with or suspected of having Crohn's Disease.

### SUMMARY

The subject matter described herein provides systems and methods for testing food intolerance in patients diagnosed with or suspected to have Crohn's Disease. One aspect of the disclosure is a test kit with for testing food intolerance in patients diagnosed with or suspected to have Crohn's Disease. The test kit includes a plurality of distinct food preparations coupled to individually addressable respective solid carriers. The plurality of distinct food preparations have an average discriminatory p-value of ≤0.07 as determined by raw p-value or an average discriminatory p-value of ≤0.10 as determined by FDR multiplicity adjusted p-value. In some embodiments, the average discriminatory p-value is determined by a process, which includes comparing assay values of a first patient test cohort that is diagnosed with or suspected of having Crohn's Disease with assay values of a second patient test cohort that is not diagnosed with or suspected of having Crohn's Disease.

Another aspect of the embodiments described herein includes a method of testing food intolerance in patients diagnosed with or suspected to have Crohn's Disease. The method includes a step of contacting a food preparation with a bodily fluid of a patient that is diagnosed with or suspected to have Crohn's Disease. The bodily fluid is associated with gender identification. In certain embodiments, the step of contacting is performed under conditions that allow IgG from the bodily fluid to bind to at least one component of the food preparation. The method continues with a step of measuring IgG bound to the at least one component of the food preparation to obtain a signal, and then comparing the signal to a gender-stratified reference value for the food preparation using the gender identification to obtain a result. Then, the method also includes a step of updating or generating a report using the result.

Another aspect of the embodiments described herein includes a method of generating a test for food intolerance in patients diagnosed with or suspected to have Crohn's Disease. The method includes a step of obtaining test results for a plurality of distinct food preparations. The test results are based on bodily fluids of patients diagnosed with or suspected to have Crohn's Disease and bodily fluids of a control group not diagnosed with or not suspected to have Crohn's Disease. The method also includes a step of stratifying the test results by gender for each of the distinct food preparations. Then the method continues with a step of assigning for a predetermined percentile rank a different cutoff value for male and female patients for each of the distinct food preparations.

Still another aspect of the embodiments described herein includes a use of a plurality of distinct food preparations coupled to individually addressable respective solid carriers in a diagnosis of Crohn's Disease. The plurality of distinct food preparations are selected based on their average discriminatory p-value of ≤0.07 as determined by raw p-value or an average discriminatory p-value of ≤0.10 as determined by FDR multiplicity adjusted p-value.

Various objects, features, aspects and advantages of the embodiments described herein will become more apparent from the following detailed description of preferred embodiments, along with the accompanying drawing figures in which like numerals represent like components.

### BRIEF DESCRIPTION OF THE DRAWINGS

FIG. 1A illustrates ELISA signal score of male Crohn's Disease patients and control tested with almond.

FIG. 1B illustrates a distribution of percentage of male Crohn's Disease subjects exceeding the 90^{th} and 95^{th} percentile tested with almond.

FIG. 1C illustrates a signal distribution in women along with the 95^{th} percentile cutoff as determined from the female control population tested with almond.

FIG. 1D illustrates a distribution of percentage of female Crohn's Disease subjects exceeding the 90^{th} and 95^{th} percentile tested with almond.

FIG. 2A illustrates ELISA signal score of male Crohn's Disease patients and control tested with apple.

FIG. 2B illustrates a distribution of percentage of male Crohn's Disease subjects exceeding the 90^{th} and 95^{th} percentile tested with apple.

FIG. 2C illustrates a signal distribution in women along with the 95^{th} percentile cutoff as determined from the female control population tested with apple.

FIG. 2D illustrates a distribution of percentage of female Crohn's Disease subjects exceeding the 90^{th} and 95^{th} percentile tested with apple.

FIG. 3A illustrates ELISA signal score of male Crohn's Disease patients and control tested with avocado.

FIG. 3B illustrates a distribution of percentage of male Crohn's Disease subjects exceeding the 90^{th} and 95^{th} percentile tested with avocado.

FIG. 3C illustrates a signal distribution in women along with the 95^{th} percentile cutoff as determined from the female control population tested with avocado.

FIG. 3D illustrates a distribution of percentage of female Crohn's Disease subjects exceeding the 90^{th} and 95^{th} percentile tested with avocado.

FIG. 4A illustrates ELISA signal score of male Crohn's Disease patients and control tested with barley.

FIG. 4B illustrates a distribution of percentage of male Crohn's Disease subjects exceeding the 90^{th} and 95^{th} percentile tested with barley.

FIG. 4C illustrates a signal distribution in women along with the 95^{th} percentile cutoff as determined from the female control population tested with barley.

FIG. 4D illustrates a distribution of percentage of female Crohn's Disease subjects exceeding the 90^{th} and 95^{th} percentile tested with barley.

FIG. 5A illustrates distributions of Crohn's Disease subjects by number of foods that were identified as trigger foods at the 90^{th} percentile.

FIG. 5B illustrates distributions of Crohn's Disease subjects by number of foods that were identified as trigger foods at the 95^{th} percentile.

FIG. 6A illustrates a box and whisker plot of data shown in Table 5A.

FIG. 6B illustrates a notched box and whisker plot of data shown in Table 5A.

FIG. 6C illustrates a box and whisker plot of data shown in Table 5B.

FIG. 6D illustrates a notched box and whisker plot of data shown in Table 5B.

FIG. 7A illustrates the ROC curve corresponding to the statistical data shown in Table 12A.

FIG. 7B illustrates the ROC curve corresponding to the statistical data shown in Table 12B.

### BRIEF DESCRIPTION OF THE TABLES

Table 1 shows a list of food items from which food preparations can be prepared.

Table 2 shows statistical data of foods ranked according to 2-tailed FDR multiplicity-adjusted p-values.

Table 3 shows statistical data of ELISA score by food and gender.

Table 4 shows cutoff values of foods for a predetermined percentile rank.

Table 5A shows raw data of Crohn's Disease patients and control with number of positive results based on the 90^{th} percentile.

Table 5B shows raw data of Crohn's Disease patients and control with number of positive results based on the 95^{th} percentile.

Table 6A shows statistical data summarizing the raw data of Crohn's Disease patient populations shown in Table 5A.

Table 6B shows statistical data summarizing the raw data of Crohn's Disease patient populations shown in Table 5B.

Table 7A shows statistical data summarizing the raw data of control populations shown in Table 5A.

Table 7B shows statistical data summarizing the raw data of control populations shown in Table 5B.

Table 8A shows statistical data summarizing the raw data of Crohn's Disease patient populations shown in Table 5A transformed by logarithmic transformation.

Table 8B shows statistical data summarizing the raw data of Crohn's Disease patient populations shown in Table 5B transformed by logarithmic transformation.

Table 9A shows statistical data summarizing the raw data of control populations shown in Table 5A transformed by logarithmic transformation.

Table 9B shows statistical data summarizing the raw data of control populations shown in Table 5B transformed by logarithmic transformation.

Table 10A shows statistical data of an independent T-test to compare the geometric mean number of positive foods between the Crohn's Disease and non-Crohn's Disease samples based on the 90^{th} percentile.

Table 10B shows statistical data of an independent T-test to compare the geometric mean number of positive foods between the Crohn's Disease and non-Crohn's Disease samples based on the 95^{th} percentile.

Table 11A shows statistical data of a Mann-Whitney test to compare the geometric mean number of positive foods between the Crohn's Disease and non-Crohn's Disease samples based on the 90^{th} percentile.

Table 11B shows statistical data of a Mann-Whitney test to compare the geometric mean number of positive foods between the Crohn's Disease and non-Crohn's Disease samples based on the 95^{th} percentile.

Table 12A shows statistical data of a Receiver Operating Characteristic (ROC) curve analysis of data shown in Tables 5A-11A.

Table 12B shows statistical data of a Receiver Operating Characteristic (ROC) curve analysis of data shown in Tables 5B-11B.

Table 13A shows a statistical data of performance metrics in predicting Crohn's Disease status among female patients from number of positive foods based on the 90^{th} percentile.

Table 13B shows a statistical data of performance metrics in predicting Crohn's Disease status among male patients from number of positive foods based on the 90^{th} percentile.

Table 14A shows a statistical data of performance metrics in predicting Crohn's Disease status among female patients from number of positive foods based on the 95^{th} percentile.

Table 14B shows a statistical data of performance metrics in predicting Crohn's Disease status among male patients from number of positive foods based on the 95^{th} percentile.

### DETAILED DESCRIPTION

The inventors have discovered that food preparations used in food tests to identify trigger foods in patients diagnosed with or suspected to have Crohn's Disease are not equally well predictive and/or associated with Crohn's Disease/Crohn's Disease symptoms. Indeed, various experiments have revealed that among a wide variety of food items certain food items are highly predictive/associated with Crohn's Disease whereas others have no statistically significant association with Crohn's Disease.

Even more unexpectedly, the inventors discovered that in addition to the high variability of food items, gender variability with respect to response in a test plays a substantial role in the determination of association or a food item with Crohn's Disease. Consequently, based on the inventors' findings and further contemplations, test kits and methods are now presented with substantially higher predictive power in the choice of food items that could be eliminated for reduction of Crohn's Disease signs and symptoms.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

In some embodiments, the numbers expressing quantities or ranges, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements. Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

In one aspect, the inventors therefore contemplate a test kit or test panel that is suitable for testing food intolerance in patients where the patient is diagnosed with or suspected to have Crohn's Disease. Most preferably, such test kit or panel will include a plurality of distinct food preparations (e.g., raw or processed extract, preferably aqueous extract with optional co-solvent, which may or may not be filtered) that are coupled to individually addressable respective solid carriers (e.g., in a form of an array or a micro well plate), wherein the distinct food preparations have an average discriminatory p-value of ≤0.07 as determined by raw p-value or an average discriminatory p-value of ≤0.10 as determined by FDR multiplicity adjusted p-value.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements. Moreover, and unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

While not limiting to the inventive subject matter, food preparations will typically be drawn from foods generally known or suspected to trigger signs or symptoms of Crohn's Disease. Particularly suitable food preparations may be identified by the experimental procedures outlined below. Thus, it should be appreciated that the food items need not be limited to the items described herein, but that all items are contemplated that can be identified by the methods presented herein. Therefore, exemplary food preparations include at least two, at least four, at least eight, or at least 12 food preparations prepared from foods 1-83 of Table 2. Still further especially contemplated food items and food additives from which food preparations can be prepared are listed in Table 1.

Using bodily fluids from patients diagnosed with or suspected to have Crohn's Disease and healthy control group individuals (i.e., those not diagnosed with or not suspected to have Crohn's Disease), numerous additional food items may be identified. Preferably, such identified food items will have high discriminatory power and as such have a p-value of ≤0.15, more preferably ≤0.10, and most preferably ≤0.05 as determined by raw p-value, and/or a p-value of ≤0.10, more preferably ≤0.08, and most preferably ≤0.07 as determined by False Discovery Rate (FDR) multiplicity adjusted p-value.

In certain embodiments, such identified food preparations will have high discriminatory power and, as such, will have a p-value of ≤0.15, ≤0.10, or even≤0.05 as determined by raw p-value, and/or a p-value of ≤0.10, ≤0.08, or even≤0.07 as determined by False Discovery Rate (FDR) multiplicity adjusted p-value.

Therefore, where a panel has multiple food preparations, it is contemplated that the plurality of distinct food preparations has an average discriminatory p-value of ≤0.05 as determined by raw p-value or an average discriminatory p-value of ≤0.08 as determined by FDR multiplicity adjusted p-value, or even more preferably an average discriminatory p-value of ≤ 0.025 as determined by raw p-value or an average discriminatory p-value of ≤0.07 as determined by FDR multiplicity adjusted p-value. In further preferred aspects, it should be appreciated that the FDR multiplicity adjusted p-value may be adjusted for at least one of age and gender, and most preferably adjusted for both age and gender. On the other hand, where a test kit or panel is stratified for use with a single gender, it is also contemplated that in a test kit or panel at least 50% (and more typically 70% or all) of the plurality of distinct food preparations, when adjusted for a single gender, have an average discriminatory p-value of ≤ 0.07 as determined by raw p-value or an average discriminatory p-value of ≤0.10 as determined by FDR multiplicity adjusted p-value. Furthermore, it should be appreciated that other stratifications (e.g., dietary preference, ethnicity, place of residence, genetic predisposition or family history, etc.) are also contemplated, and the person of ordinary skill in the art (PHOSITA) will be readily appraised of the appropriate choice of stratification.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Of course, it should be noted that the particular format of the test kit or panel may vary considerably and contemplated formats include micro well plates, dip sticks, membrane-bound arrays, etc. Consequently, the solid carrier to which the food preparations are coupled may include wells of a multiwell plate, a bead (e.g., color-coded or magnetic), or an adsorptive film (e.g., nitrocellulose or micro/nanoporous polymeric film), or an electrical sensor (e.g., a printed copper sensor or microchip).

Consequently, the inventors also contemplate a method of testing food intolerance in patients that are diagnosed with or suspected to have Crohn's Disease. Most typically, such methods will include a step of contacting a food preparation with a bodily fluid (e.g., whole blood, plasma, serum, saliva, or a fecal suspension) of a patient that is diagnosed with or suspected to have Crohn's Disease, and wherein the bodily fluid is associated with a gender identification. As noted before, the step of contacting is preferably performed under conditions that allow IgG (or IgE or IgA or IgM) from the bodily fluid to bind to at least one component of the food preparation, and the IgG bound to the component(s) of the food preparation are then quantified/measured to obtain a signal. In some embodiments, the signal is then compared against a gender-stratified reference value (e.g., at least a 90th percentile value) for the food preparation using the gender identification to obtain a result, which is then used to update or generate a report (e.g., written medical report; oral report of results from doctor to patient; written or oral directive from physician based on results).

In certain embodiments, such methods will not be limited to a single food preparation, but will employ multiple different food preparations. As noted before, suitable food preparations can be identified using various methods as described below, however, especially preferred food preparations include foods 1-83 of Table 2, and/or items of Table 1. As also noted above, it is generally preferred that at least some, or all of the different food preparations have an average discriminatory p-value of ≤0.07 (or ≤0.05, or ≤0.025) as determined by raw p-value, and/or or an average discriminatory p-value of ≤0.10 (or ≤0.08, or ≤0.07) as determined by FDR multiplicity adjusted p-value.

While in certain embodiments food preparations are prepared from single food items as crude extracts, or crude filtered extracts, it is contemplated that food preparations can be prepared from mixtures of a plurality of food items (e.g., a mixture of citrus comprising lemon, orange, and a grapefruit, a mixture of yeast comprising baker's yeast and brewer's yeast, a mixture of rice comprising a brown rice and white rice, a mixture of sugars comprising honey, malt, and cane sugar. In some embodiments, it is also contemplated that food preparations can be prepared from purified food antigens or recombinant food antigens.

As it is generally preferred that the food preparation is immobilized on a solid surface (typically in an addressable manner), it is contemplated that the step of measuring the IgG or other type of antibody bound to the component of the food preparation is performed via an ELISA test. Exemplary solid surfaces include, but are not limited to, wells in a multiwell plate, such that each food preparation may be isolated to a separate microwell. In certain embodiments, the food preparation will be coupled to, or immobilized on, the solid surface. In other embodiments, the food preparation(s) will be coupled to a molecular tag that allows for binding to human immunoglobulins (e.g., IgG) in solution.

Viewed from a different perspective, the inventors also contemplate a method of generating a test for food intolerance in patients diagnosed with or suspected to have Crohn's Disease. Because the test is applied to patients already diagnosed with or suspected to have Crohn's Disease, the authors do not contemplate that the method has a diagnostic purpose. Instead, the method is for identifying triggering food items among already diagnosed or suspected Crohn's Disease patients. Such test will typically include a step of obtaining one or more test results (e.g., ELISA) for various distinct food preparations, wherein the test results are based on bodily fluids (e.g., blood saliva, fecal suspension) of patients diagnosed with or suspected to have Crohn's Disease and bodily fluids of a control group not diagnosed with or not suspected to have Crohn's Disease. Most preferably, the test results are then stratified by gender for each of the distinct food preparations, a different cutoff value for male and female patients for each of the distinct food preparations (e.g., cutoff value for male and female patients has a difference of at least 10% (abs)) is assigned for a predetermined percentile rank (e.g., 90th or 95th percentile).

As noted earlier, and while not limiting to the inventive subject matter, it is contemplated that the distinct food preparations include at least two (or six, or ten, or 15) food preparations prepared from food items selected from the group consisting of foods 1-83 of Table 2, and/or items of Table 1. On the other hand, where new food items are tested, it should be appreciated that the distinct food preparations include a food preparation prepared from a food items other than foods 1-83 of Table 2. Regardless of the particular choice of food items, it is generally preferred however, that the distinct food preparations have an average discriminatory p-value of ≤0.07 (or ≤0.05, or ≤0.025) as determined by raw p-value or an average discriminatory p-value of≤0.10 (or ≤0.08, or≤0.07) as determined by FDR multiplicity adjusted p-value. Exemplary aspects and protocols, and considerations are provided in the experimental description below.

Thus, it should be appreciated that by having a high-confidence test system as described herein, the rate of false-positive and false negatives can be significantly reduced, and especially where the test systems and methods are gender stratified or adjusted for gender differences as shown below. Such advantages have heretofore not been realized and it is expected that the systems and methods presented herein will substantially increase the predictive power of food sensitivity tests for patients diagnosed with or suspected to have Crohn's Disease.

### Experiments

General Protocol for food preparation generation: Commercially available food extracts (available from Biomerica Inc., 17571 Von Karman Ave, Irvine, CA 92614) prepared from the edible portion of the respective raw foods were used to prepare ELISA plates following the manufacturer's instructions.

For some food extracts, the inventors expect that food extracts prepared with specific procedures to generate food extracts provides more superior results in detecting elevated IgG reactivity in Crohn's Disease patients compared to commercially available food extracts. For example, for grains and nuts, a three-step procedure of generating food extracts is preferred. The first step is a defatting step. In this step, lipids from grains and nuts are extracted by contacting the flour of grains and nuts with a non-polar solvent and collecting residue. Then, the defatted grain or nut flour are extracted by contacting the flour with elevated pH to obtain a mixture and removing the solid from the mixture to obtain the liquid extract. Once the liquid extract is generated, the liquid extract is stabilized by adding an aqueous formulation. In a preferred embodiment, the aqueous formulation includes a sugar alcohol, a metal chelating agent, protease inhibitor, mineral salt, and buffer component 20-50 mM of buffer from 4-9 pH. This formulation allowed for long term storage at −70° C. and multiple freeze-thaws without a loss of activity.

For another example, for meats and fish, a two step procedure of generating food extract is preferred. The first step is an extraction step. In this step, extracts from raw, uncooked meats or fish are generated by emulsifying the raw, uncooked meats or fish in an aqueous buffer formulation in a high impact pressure processor. Then, solid materials are removed to obtain liquid extract. Once the liquid extract is generated, the liquid extract is stabilized by adding an aqueous formulation. In a preferred embodiment, the aqueous formulation includes a sugar alcohol, a metal chelating agent, protease inhibitor, mineral salt, and buffer component 20-50 mM of buffer from 4-9 pH. This formulation allowed for long term storage at −70° C. and multiple freeze-thaws without a loss of activity.

For still another example, for fruits and vegetables, a two step procedure of generating food extract is preferred. The first step is an extraction step. In this step, liquid extracts from fruits or vegetables are generated using an extractor (e.g., masticating juicer, etc) to pulverize foods and extract juice. Then, solid materials are removed to obtain liquid extract. Once the liquid extract is generated, the liquid extract is stabilized by adding an aqueous formulation. In a preferred embodiment, the aqueous formulation includes a sugar alcohol, a metal chelating agent, protease inhibitor, mineral salt, and buffer component 20-50 mM of buffer from 4-9 pH. This formulation allowed for long term storage at −70° C. and multiple freeze-thaws without a loss of activity.

Blocking of ELISA plates: To optimize signal to noise, plates will be blocked with a proprietary blocking buffer. In a preferred embodiment, the blocking buffer includes 20-50 mM of buffer from 4-9 pH, a protein of animal origin and a short chain alcohol. Other blocking buffers, including several commercial preparations, can be attempted but may not provide adequate signal to noise and low assay variability required.

ELISA preparation and sample testing: Food antigen preparations were immobilized onto respective microtiter wells following the manufacturer's instructions. For the assays, the food antigens were allowed to react with antibodies present in the patients' serum, and excess serum proteins were removed by a wash step. For detection of IgG antibody binding, enzyme labeled anti-IgG antibody conjugate was allowed to react with antigen-antibody complex. A color was developed by the addition of a substrate that reacts with the coupled enzyme. The color intensity was measured and is directly proportional to the concentration of IgG antibody specific to a particular food antigen.

Methodology to determine ranked food list in order of ability of ELISA signals to distinguish Crohn's Disease from control subjects: Out of an initial selection (e.g., 100 food items, or 150 food items, or even more), samples can be eliminated prior to analysis due to low consumption in an intended population. In addition, specific food items can be used as being representative of a larger generic food group, especially where prior testing has established a correlation among different species within a generic group (most preferably in both genders, but also suitable for correlation for a single gender). For example, green pepper could be dropped in favor of chili pepper as representative of the “pepper” food group, or sweet potato could be dropped in favor of potato as representative of the “potato” food group. In further preferred aspects, the final list foods will be shorter than 50 food items, and more preferably equal or less than of 40 food items.

Since the foods ultimately selected for the food intolerance panel will not be specific for a particular gender, a gender-neutral food list is necessary. Since the observed sample will be at least initially imbalanced by gender (e.g., Controls: 40% female, Crohn's Disease: 58% female), differences in ELISA signal magnitude strictly due to gender will be removed by modeling signal scores against gender using a two-sample t-test and storing the residuals for further analysis. For each of the tested foods, residual signal scores will be compared between Crohn's Disease and controls using a permutation test on a two-sample t-test with a relative high number of resamplings (e.g., >1,000, more preferably >10,000, even more preferably >50,000). The Satterthwaite approximation can then be used for the denominator degrees of freedom to account for lack of homogeneity of variances, and the 2-tailed permuted p-value will represent the raw p-value for each food. False Discovery Rates (FDR) among the comparisons, will be adjusted by any acceptable statistical procedures (e.g., Benjamini-Hochberg, Family-wise Error Rate (FWER), Per Comparison Error Rate (PCER), etc.).

Foods were then ranked according to their 2-tailed FDR multiplicity-adjusted p-values. Foods with adjusted p-values equal to or lower than the desired FDR threshold are deemed to have significantly higher signal scores among Crohn's Disease than control subjects and therefore deemed candidates for inclusion into a food intolerance panel. A typical result that is representative of the outcome of the statistical procedure is provided in Table 2. Here the ranking of foods is according to 2-tailed permutation T-test p-values with FDR adjustment.

Based on earlier experiments (data not shown here, see U.S. 62/327,917), the inventors contemplate that even for the same food preparation tested, the ELISA score for at least several food items will vary dramatically, and exemplary raw data are provided in Table 3. As should be readily appreciated, data unstratified by gender will therefore lose significant explanatory power where the same cutoff value is applied to raw data for male and female data. To overcome such disadvantage, the inventors therefore contemplate stratification of the data by gender as described below.

Statistical Method for Cutpoint Selection for each Food: The determination of what ELISA signal scores would constitute a “positive” response can be made by summarizing the distribution of signal scores among the Control subjects. For each food, Crohn's Disease subjects who have observed scores greater than or equal to selected quantiles of the Control subject distribution will be deemed “positive”. To attenuate the influence of any one subject on cutpoint determination, each food-specific and gender-specific dataset will be bootstrap resampled 1000 times. Within each bootstrap replicate, the 90th and 95th percentiles of the Control signal scores will be determined. Each Crohn's Disease subject in the bootstrap sample will be compared to the 90th and 95% percentiles to determine whether he/she had a “positive” response. The final 90th and 95th percentile-based cutpoints for each food and gender will be computed as the average 90th and 95th percentiles across the 1000 samples. The number of foods for which each Crohn's Disease subject will be rated as “positive” was computed by pooling data across foods. Using such method, the inventors will be now able to identify cutoff values for a predetermined percentile rank that in most cases was substantially different as can be taken from Table 4.

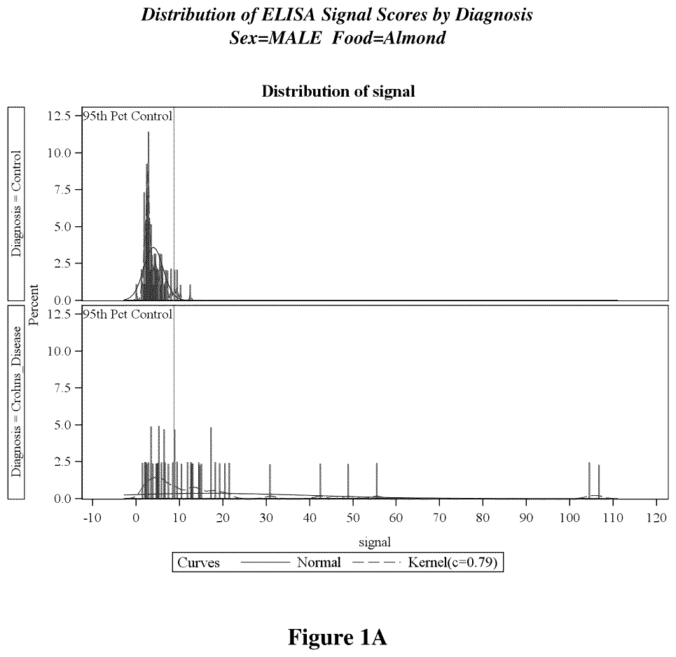

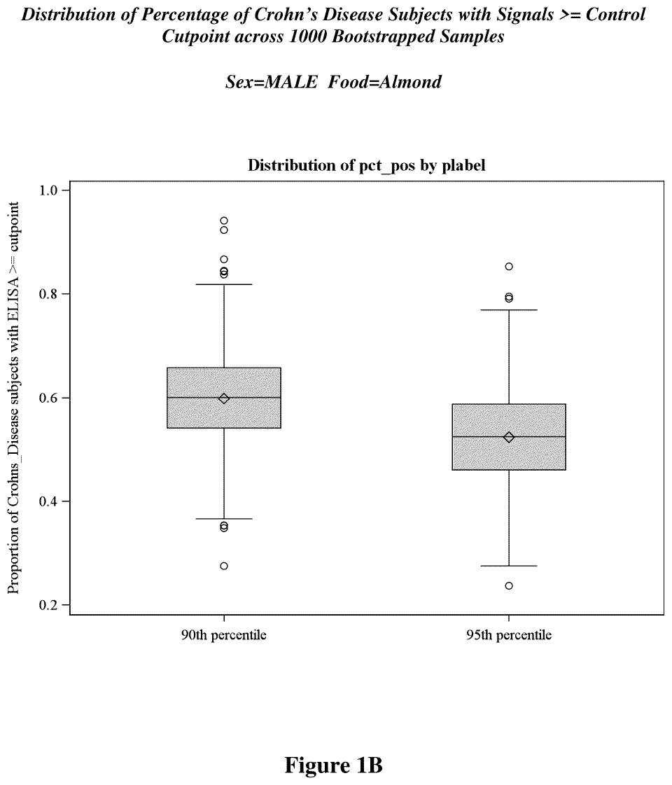

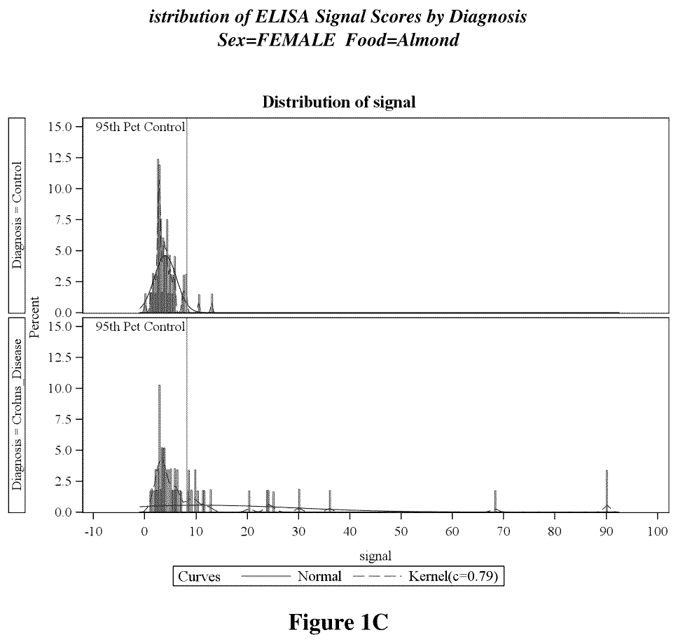

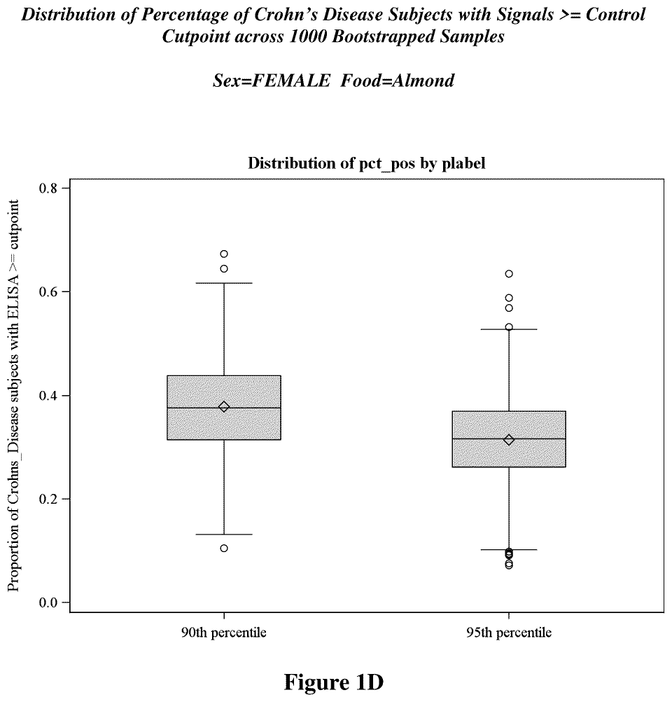

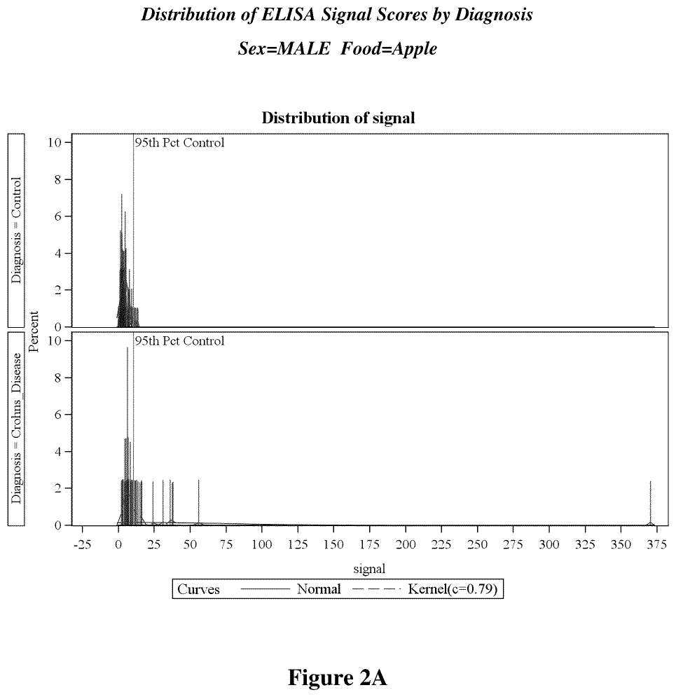

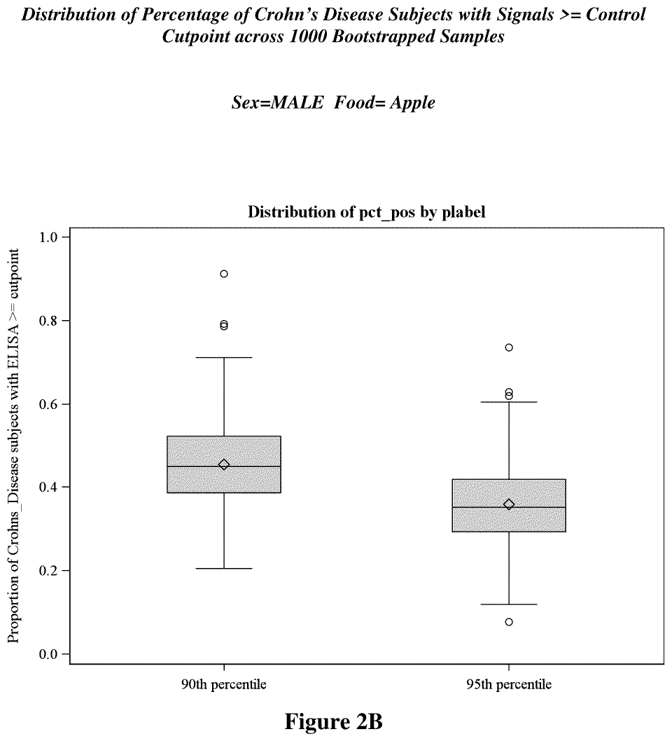

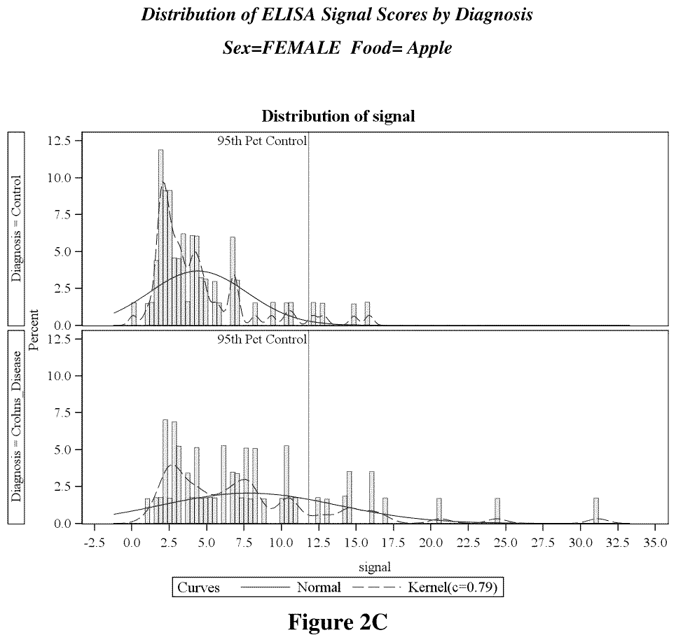

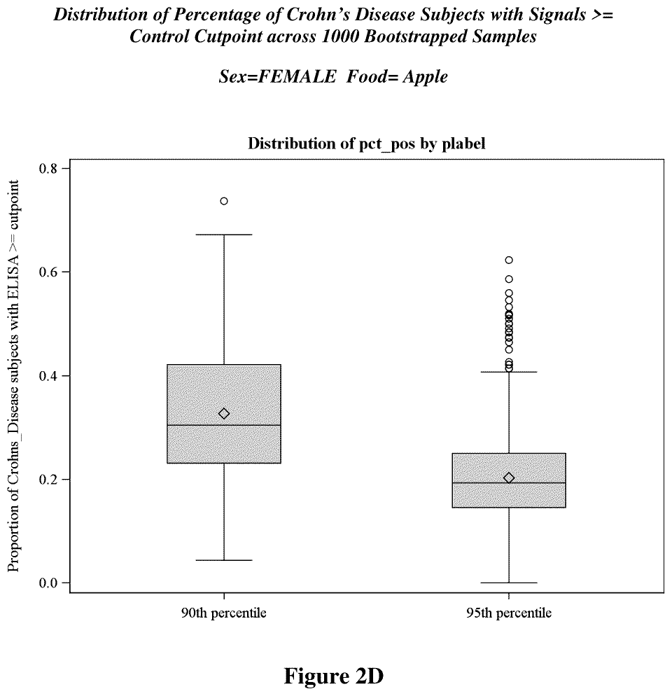

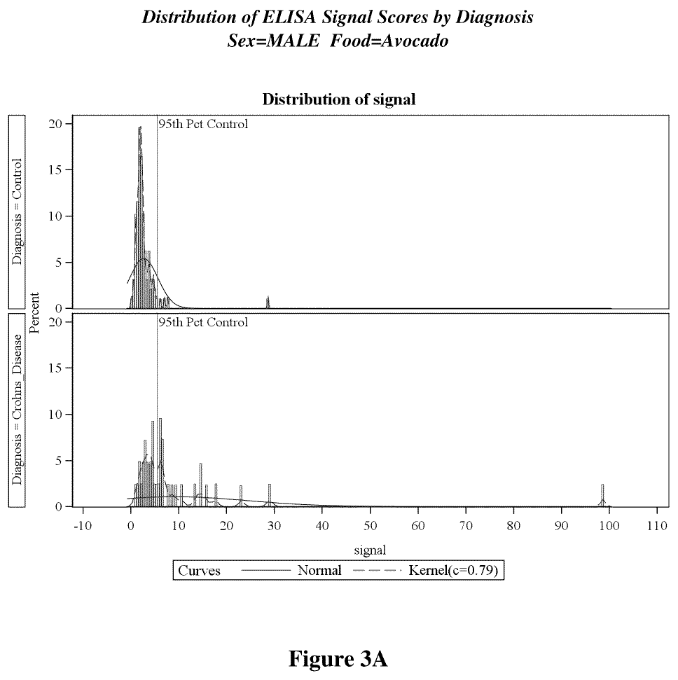

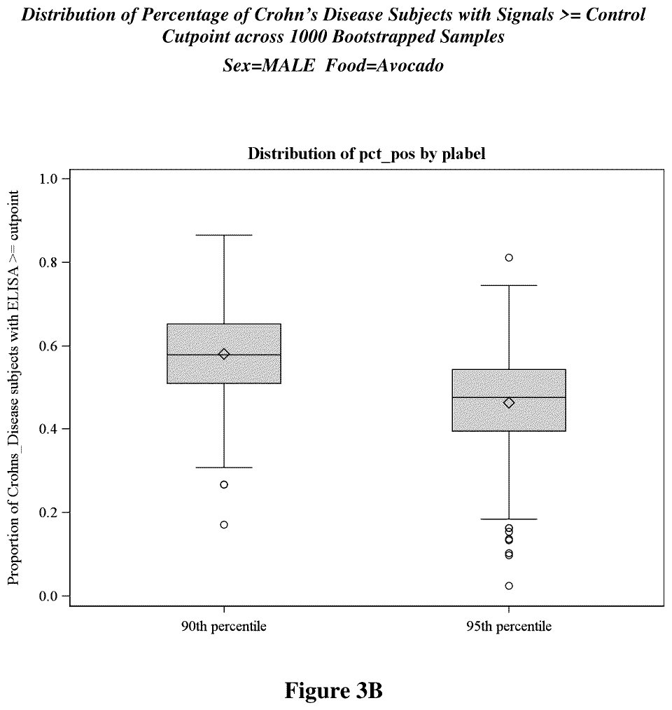

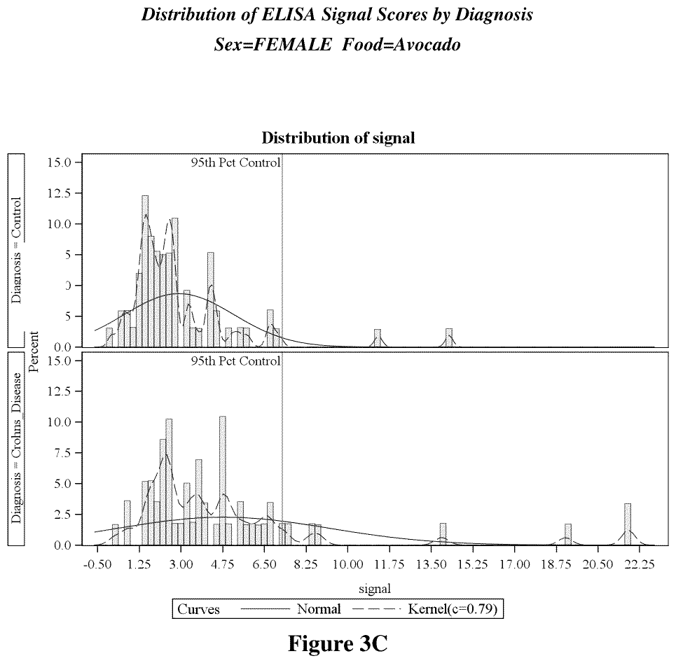

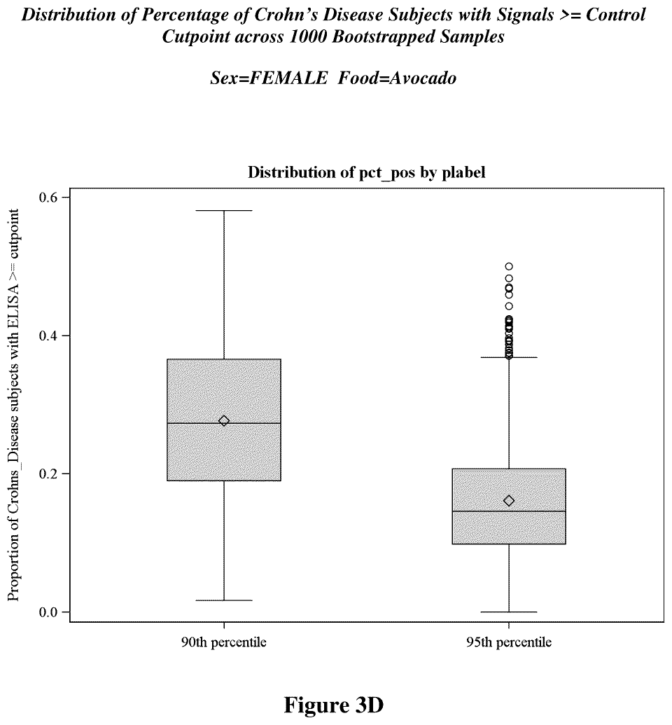

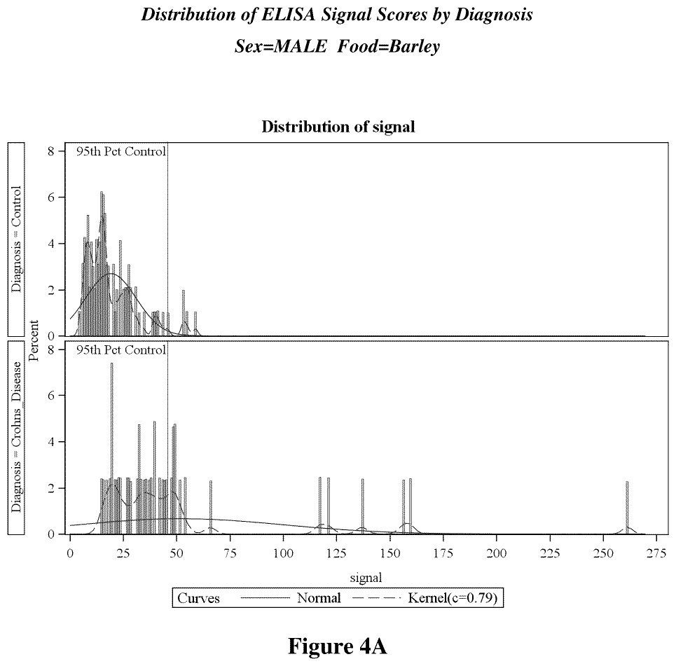

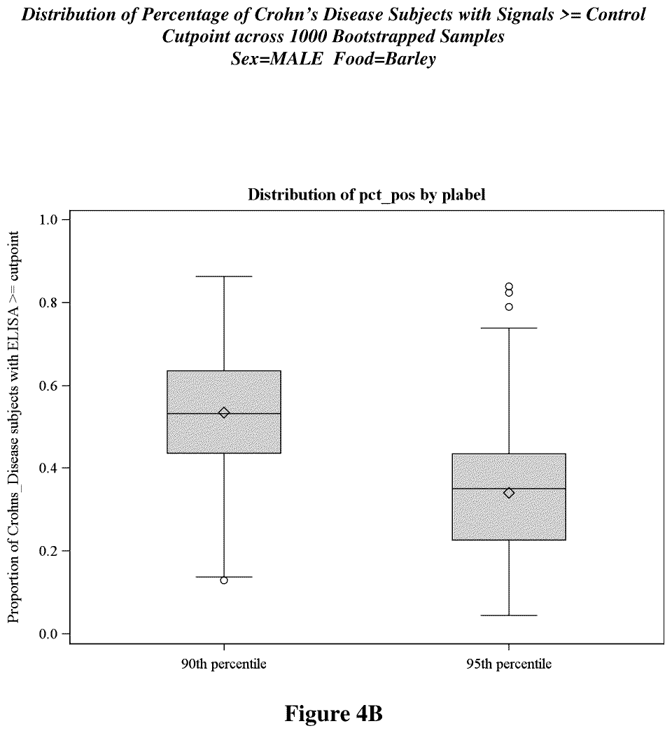

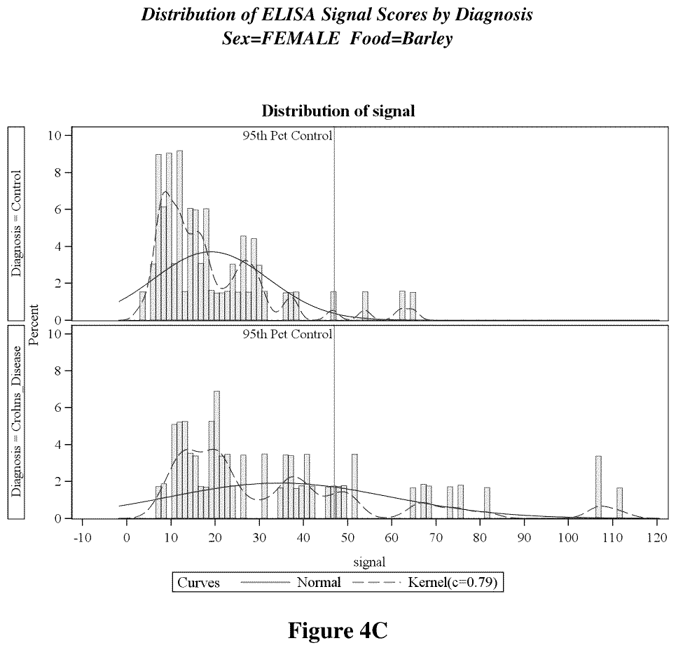

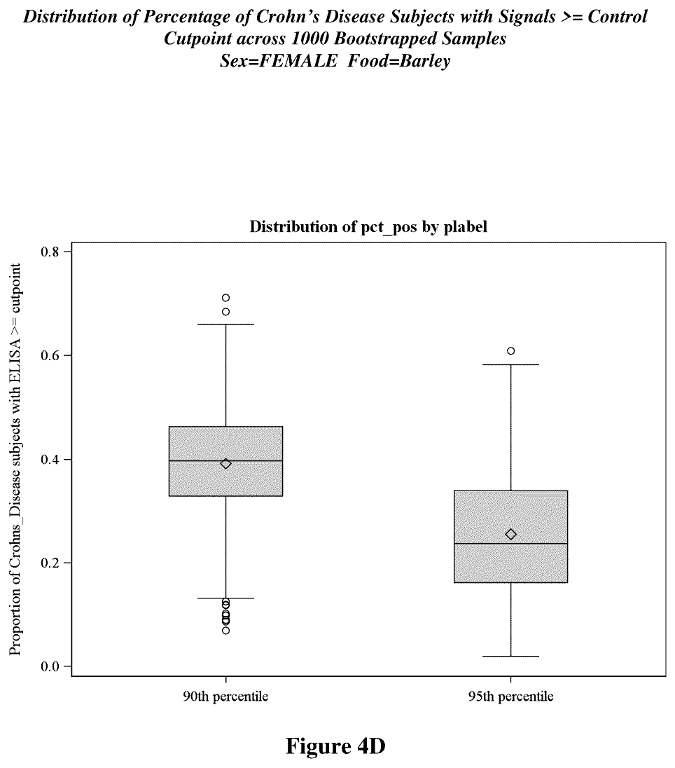

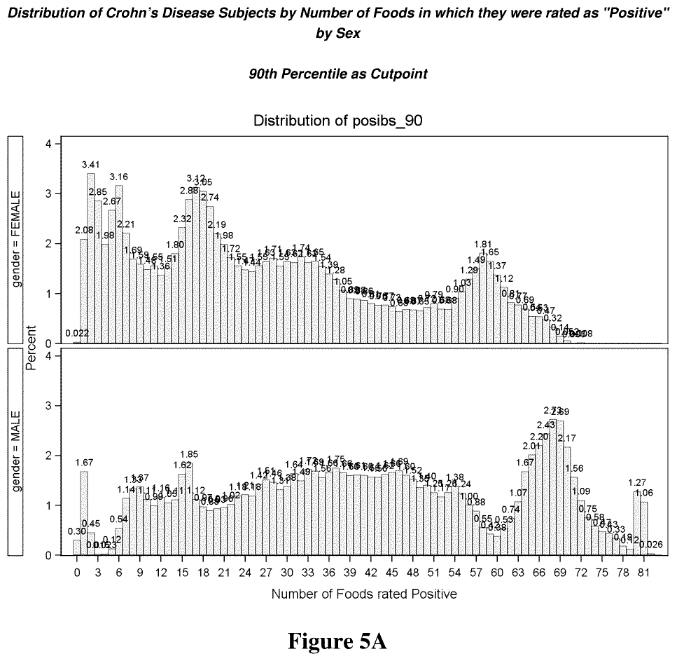

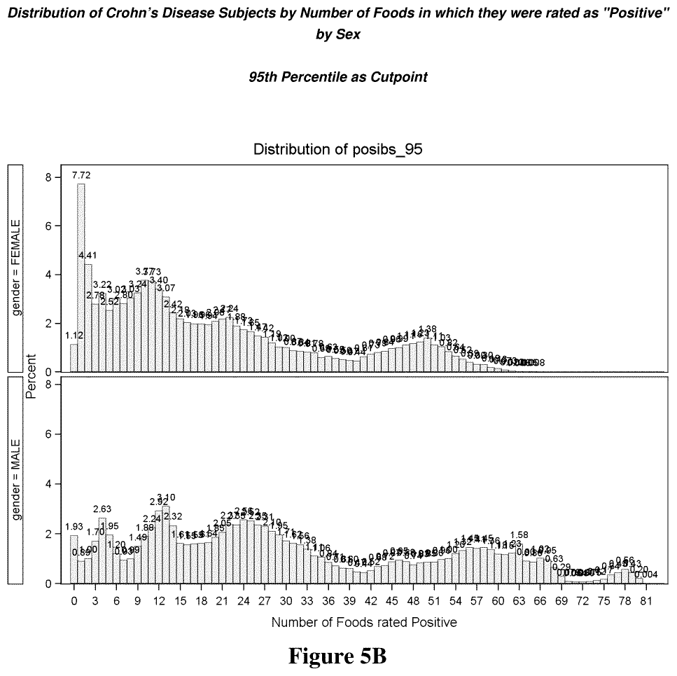

Typical examples for the gender difference in IgG response in blood with respect to almond is shown in FIGS. 1A-1D, where FIG. 1A shows the signal distribution in men along with the 95^{th} percentile cutoff as determined from the male control population. FIG. 1B shows the distribution of percentage of male Crohn's Disease subjects exceeding the 90^{th} and 95^{th} percentile, while FIG. 1C shows the signal distribution in women along with the 95^{th} percentile cutoff as determined from the female control population. FIG. 1D shows the distribution of percentage of female Crohn's Disease subjects exceeding the 90^{th} and 95^{th} percentile. In the same fashion, FIGS. 2A-2D exemplarily depict the differential response to apple, FIGS. 3A-3D exemplarily depict the differential response to avocado, and FIGS. 4A-4D exemplarily depict the differential response to barley. FIGS. 5A-5B show the distribution of Crohn's Disease subjects by number of foods that were identified as trigger foods at the 90^{th} percentile (5A) and 95^{th} percentile (5B). Inventors contemplate that regardless of the particular food items, male and female responses will be notably distinct.

It should be noted that nothing in the art have provided any predictable food groups related to Crohn's Disease that is gender-stratified. Thus, a discovery of food items that show distinct responses by gender is a surprising result, which could not be obviously expected in view of all previously available arts. In other words, selection of food items based on gender stratification provides an unexpected technical effect such that statistical significances for particular food items as triggering food among male or female Crohn's Disease patients have been significantly improved.

Normalization of IgG Response Data: While the raw data of the patient's IgG response results can be used to compare strength of response among given foods, it is also contemplated that the IgG response results of a patient are normalized and indexed to generate unit-less numbers for comparison of relative strength of response to a given food. For example, one or more of a patient's food specific IgG results (e.g., IgG specific to orange and IgG specific to malt) can be normalized to the patient's total IgG. The normalized value of the patient's IgG specific to orange can be 0.1 and the normalized value of the patient's IgG specific to malt can be 0.3. In this scenario, the relative strength of the patient's response to malt is three times higher compared to orange. Then, the patient's sensitivity to malt and orange can be indexed as such.

In other examples, one or more of a patient's food specific IgG results (e.g., IgG specific to shrimp and IgG specific to pork) can be normalized to the global mean of that patient's food specific IgG results. The global means of the patient's food specific IgG can be measured by total amount of the patient's food specific IgG. In this scenario, the patient's specific IgG to shrimp can be normalized to the mean of patient's total food specific IgG (e.g., mean of IgG levels to shrimp, pork, Dungeness crab, chicken, peas, etc.). However, it is also contemplated that the global means of the patient's food specific IgG can be measured by the patient's IgG levels to a specific type of food via multiple tests. If the patient have been tested for his sensitivity to shrimp five times and to pork seven times previously, the patient's new IgG values to shrimp or to pork are normalized to the mean of five-times test results to shrimp or the mean of seven-times test results to pork. The normalized value of the patient's IgG specific to shrimp can be 6.0 and the normalized value of the patient's IgG specific to pork can be 1.0. In this scenario, the patient has six times higher sensitivity to shrimp at this time compared to his average sensitivity to shrimp, but substantially similar sensitivity to pork. Then, the patient's sensitivity to shrimp and pork can be indexed based on such comparison.

Methodology to determine the subset of Crohn's Disease patients with food sensitivities that underlie Crohn's Disease: While it is suspected that food sensitivities plays a substantial role in signs and symptoms of Crohn's Disease, some Crohn's Disease patients may not have food sensitivities that underlie Crohn's Disease. Those patients would not be benefit from dietary intervention to treat signs and symptoms of Crohn's Disease. To determine the subset of such patients, body fluid samples of Crohn's Disease patients and non-Crohn's Disease patients can be tested with ELISA test using test devices with up to 83 food samples.

Table 5A and Table 5B provide exemplary raw data. As should be readily appreciated, the data indicate number of positive results out of 83 sample foods based on 90^{th} percentile value (Table 5A) or 95^{th} percentile value (Table 5B). The first column is Crohn's Disease (n=100); second column is non-Crohn's Disease (n=163) by ICD-10 code. Average and median number of positive foods was computed for Crohn's Disease and non-Crohn's Disease patients. From the raw data shown in Table 5A and Table 5B, average and standard deviation of the number of positive foods was computed for Crohn's Disease and non-Crohn's Disease patients. Additionally, the number and percentage of patients with zero positive foods was calculated for both Crohn's Disease and non-Crohn's Disease. The number and percentage of patients with zero positive foods in the Crohn's Disease population is dramatically lower than the percentage of patients with zero positive foods in the non-Crohn's Disease population (0% vs. 12.3%, respectively) based on 90^{th} percentile value (Table 5A), and the percentage of patients in the Crohn's Disease population with zero positive foods is also significantly lower (i.e. 12-fold lower) than that seen in the non-Crohn's Disease population (2% vs. 24%, respectively) based on 95^{th} percentile value (Table 5B). Thus, it can be easily appreciated that the Crohn's Disease patient having sensitivity to zero positive foods is unlikely to have food sensitivities underlying their signs and symptoms of Crohn's Disease.

Table 6A and Table 7A show exemplary statistical data summarizing the raw data of two patient populations shown in Table 5A. The statistical data includes normality, arithmetic mean, median, percentiles and 95% confidence interval (CI) for the mean and median representing number of positive foods in the Crohn's Disease population and the non-Crohn's Disease population. Table 6B and Table 7B show exemplary statistical data summarizing the raw data of two patient populations shown in Table 5B. The statistical data includes normality, arithmetic mean, median, percentiles and 95% confidence interval (CI) for the mean and median representing number of positive foods in the Crohn's Disease population and the non-Crohn's Disease population.

Table 8A and Table 9A show exemplary statistical data summarizing the raw data of two patient populations shown in Table 5A. In Tables 8A and 9A, the raw data was transformed by logarithmic transformation to improve the data interpretation. Table 8B and Table 9B show another exemplary statistical data summarizing the raw data of two patient populations shown in Table 5B. In Tables 8B and 9B, the raw data was transformed by logarithmic transformation to improve the data interpretation.

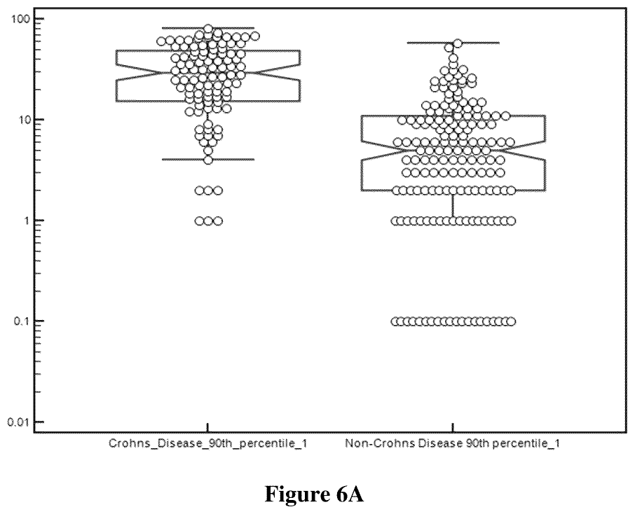

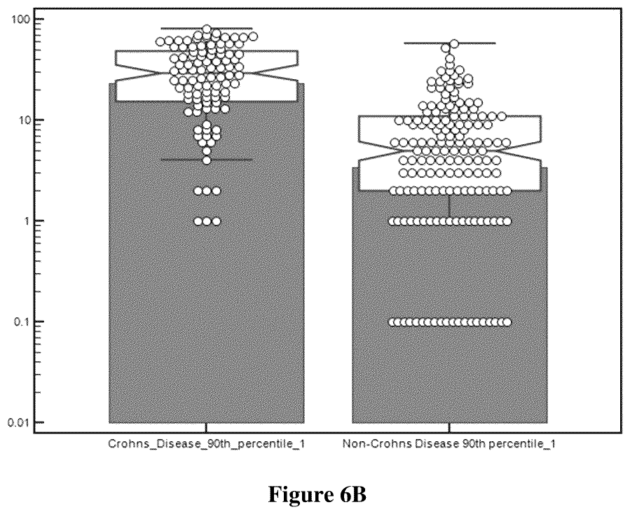

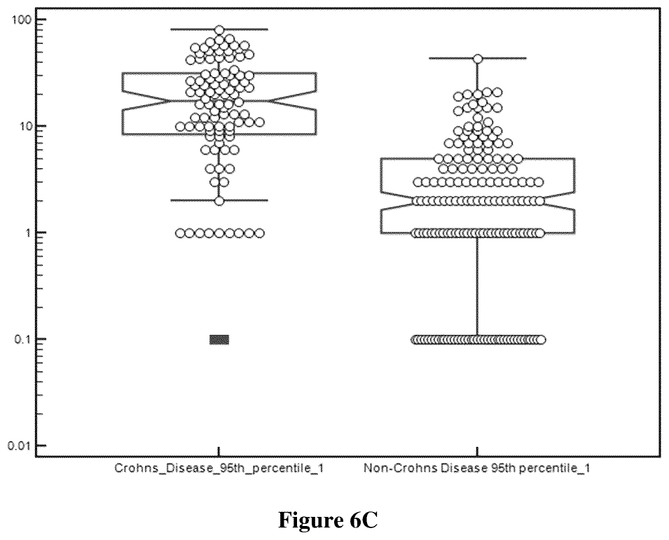

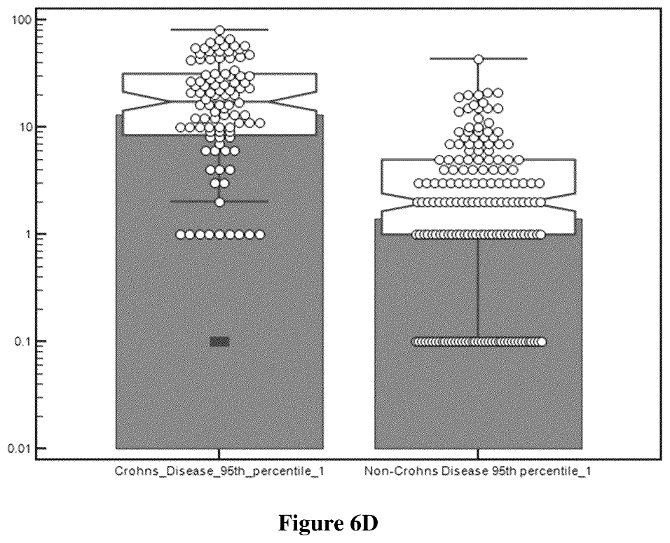

Table 10A and Table 11A show exemplary statistical data of an independent T-test (Table 10A, logarithmically transformed data) and a Mann-Whitney test (Table 11A) to compare the geometric mean number of positive foods between the Crohn's Disease and non-Crohn's Disease samples. The data shown in Table 10A and Table 11A indicate statistically significant differences in the geometric mean of positive number of foods between the Crohn's Disease population and the non-Crohn's Disease population. In both statistical tests, it is shown that the number of positive responses with 83 food samples is significantly higher in the Crohn's Disease population than in the non-Crohn's Disease population with an average discriminatory p-value of ≤0.0001. These statistical data is also illustrated as a box and whisker plot in FIG. 6A, and a notched box and whisker plot in FIG. 6B.

Table 10B and Table 11B show exemplary statistical data of an independent T-test (Table 10A, logarithmically transformed data) and a Mann-Whitney test (Table 11B) to compare the geometric mean number of positive foods between the Crohn's Disease and non-Crohn's Disease samples. The data shown in Table 10B and Table 11B indicate statistically significant differences in the geometric mean of positive number of foods between the Crohn's Disease population and the non-Crohn's Disease population. In both statistical tests, it is shown that the number of positive responses with 83 food samples is significantly higher in the Crohn's Disease population than in the non-Crohn's Disease population with an average discriminatory p-value of ≤0.0001. These statistical data is also illustrated as a box and whisker plot in FIG. 6C, and a notched box and whisker plot in FIG. 6D.

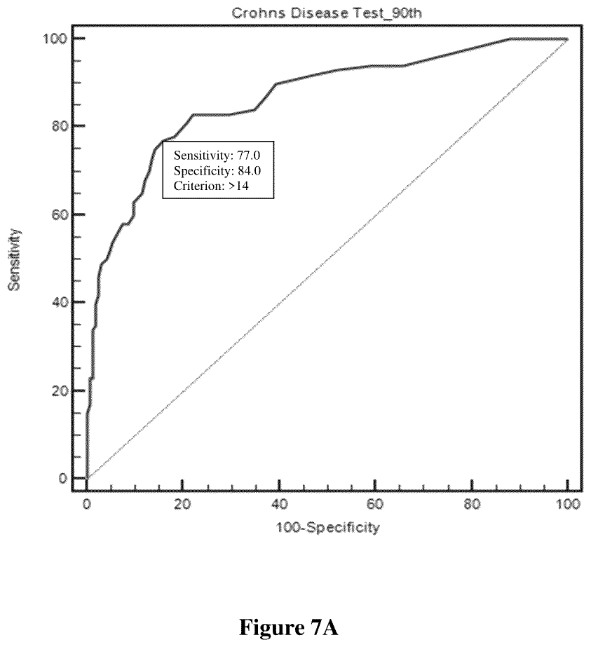

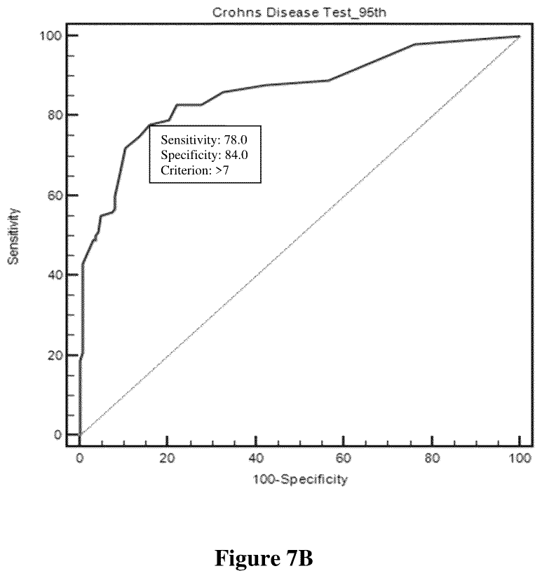

Table 12A shows exemplary statistical data of a Receiver Operating Characteristic (ROC) curve analysis of data shown in Tables 5A-11A to determine the diagnostic power of the test used in Table 5 at discriminating Crohn's Disease from non-Crohn's Disease subjects. When a cutoff criterion of more than 14 positive foods is used, the test yields a data with 77% sensitivity and 84% specificity, with an area under the curve (AUROC) of 0.865. The p-value for the ROC is significant at a p-value of <0.0001. FIG. 7A illustrates the ROC curve corresponding to the statistical data shown in Table 12A. Because the statistical difference between the Crohn's Disease population and the non-Crohn's Disease population is significant when the test results are cut off to a positive number of 14, the number of foods for which a patient tests positive could be used as a confirmation of the primary clinical diagnosis of Crohn's Disease, and whether it is likely that food sensitivities underlies on the patient's signs and symptoms of Crohn's Disease. Therefore, the above test can be used as another ‘rule in’ test to add to currently available clinical criteria for diagnosis for Crohn's Disease.

As shown in Tables 5A-12A, and FIG. 7A, based on 90^{th} percentile data, the number of positive foods seen in Crohn's Disease vs. non-Crohn's Disease subjects is significantly different whether the geometric mean or median of the data is compared. The number of positive foods that a person has is indicative of the presence of Crohn's Disease in subjects. The test has discriminatory power to detect Crohn's Disease with 77% sensitivity and 84% specificity. Additionally, the absolute number and percentage of subjects with 0 positive foods is also very different in Crohn's Disease vs. non-Crohn's Disease subjects, with a far lower percentage of Crohn's Disease subjects (0%) having 0 positive foods than non-Crohn's Disease subjects (12.3%). The data suggests a subset of Crohn's Disease patients may have Crohn's Disease due to other factors than diet, and may not benefit from dietary restriction.

Table 12B shows exemplary statistical data of a Receiver Operating Characteristic (ROC) curve analysis of data shown in Tables 5B-11B to determine the diagnostic power of the test used in Table 5 at discriminating Crohn's Disease from non-Crohn's Disease subjects. When a cutoff criterion of more than 7 positive foods is used, the test yields a data with 78% sensitivity and 84% specificity, with an area under the curve (AUROC) of 0.863. The p-value for the ROC is significant at a p-value of <0.0001. FIG. 7B illustrates the ROC curve corresponding to the statistical data shown in Table 12B. Because the statistical difference between the Crohn's Disease population and the non-Crohn's Disease population is significant when the test results are cut off to positive number of >7, the number of foods that a patient tests positive could be used as a confirmation of the primary clinical diagnosis of Crohn's Disease, and whether it is likely that food sensitivities underlies on the patient's signs and symptoms of Crohn's Disease. Therefore, the above test can be used as another ‘rule in’ test to add to currently available clinical criteria for diagnosis for Crohn's Disease.

As shown in Tables 5B-12B, and FIG. 7B, based on 95^{th} percentile data, the number of positive foods seen in Crohn's Disease vs. non-Crohn's Disease subjects is significantly different whether the geometric mean or median of the data is compared. The number of positive foods that a person has is indicative of the presence of Crohn's Disease in subjects. The test has discriminatory power to detect Crohn's Disease with 78% sensitivity and 84% specificity. Additionally, the absolute number and percentage of subjects with 0 positive foods is also very different in Crohn's Disease vs. non-Crohn's Disease subjects, with a far lower percentage of Crohn's Disease subjects (2%) having 0 positive foods than non-Crohn's Disease subjects (24%). The data suggests a subset of Crohn's Disease patients may have Crohn's Disease due to other factors than diet, and may not benefit from dietary restriction.

Method for determining distribution of per-person number of foods declared “positive”: To determine the distribution of number of “positive” foods per person and measure the diagnostic performance, the analysis will be performed with 83 food items from Table 2, which shows most positive responses to Crohn's Disease patients. To attenuate the influence of any one subject on this analysis, each food-specific and gender-specific dataset will be bootstrap resampled 1000 times. Then, for each food item in the bootstrap sample, sex-specific cutpoint will be determined using the 90th and 95th percentiles of the control population. Once the sex-specific cutpoints are determined, the sex-specific cutpoints will be compared with the observed ELISA signal scores for both control and Crohn's Disease subjects. In this comparison, if the observed signal is equal or more than the cutpoint value, then it will be determined “positive” food, and if the observed signal is less than the cutpoint value, then it will be determined “negative” food.

Once all food items were determined either positive or negative, the results of the 166 (83 foods×2 cutpoints) calls for each subject will be saved within each bootstrap replicate. Then, for each subject, 83 calls will be summed using 90^{th} percentile as cutpoint to get “Number of Positive Foods (90^{th}),” and the rest of 83 calls will be summed using 95^{th} percentile to get “Number of Positive Foods (95^{th}).” Then, within each replicate, “Number of Positive Foods (90^{th})” and “Number of Positive Foods (95^{th})” will be summarized across subjects to get descriptive statistics for each replicate as follows: 1) overall means equals to the mean of means, 2) overall standard deviation equals to the mean of standard deviations, 3) overall medial equals to the mean of medians, 4) overall minimum equals to the minimum of minimums, and 5) overall maximum equals to maximum of maximum. In this analysis, to avoid non-integer “Number of Positive Foods” when computing frequency distribution and histogram, the authors will pretend that the 1000 repetitions of the same original dataset were actually 999 sets of new subjects of the same size added to the original sample. Once the summarization of data is done, frequency distributions and histograms will be generated for both “Number of Positive Foods (90^{th})” and “Number of Positive Foods (95^{th})” for both genders and for both Crohn's Disease subjects and control subjects using programs “a_pos_foods.sas, a pos_foods_by_dx.sas”.

Method for measuring diagnostic performance: To measure diagnostic performance for each food items for each subject, we will use data of “Number of Positive Foods (90^{th})” and “Number of Positive Foods (95^{th})” for each subject within each bootstrap replicate described above. In this analysis, the cutpoint was set to 1. Thus, if a subject has one or more “Number of Positive Foods (90^{th})”, then the subject will be called “Has Crohn's Disease.” If a subject has less than one “Number of Positive Foods (90^{th})”, then the subject will be called “Does Not Have Crohn's Disease.” When all calls were made, the calls were compared with actual diagnosis to determine whether a call was a True Positive (TP), True Negative (TN), False Positive (FP), or False Negative (FN). The comparisons will be summarized across subjects to get the performance metrics of sensitivity, specificity, positive predictive value, and negative predictive value for both “Number of Positive Foods (90^{th})” and “Number of Positive Foods (95^{th})” when the cutpoint is set to 1 for each method. Each (sensitivity, 1-specificity) pair becomes a point on the ROC curve for this replicate.

To increase the accuracy, the analysis above will be repeated by incrementing cutpoint from 2 up to 83, and repeated for each of the 1000 bootstrap replicates. Then the performance metrics across the 1000 bootstrap replicates will be summarized by calculating averages using a program “t_pos_foods_by_dx.sas”. The results of diagnostic performance for female and male are shown in Tables 13A and 13B (90th percentile) and Tables 14A and 14B (95th percentile).

Of course, it should be appreciated that certain variations in the food preparations may be made without altering the inventive subject matter presented herein. For example, where the food item was yellow onion, that item should be understood to also include other onion varieties that were demonstrated to have equivalent activity in the tests. Indeed, the inventors have noted that for each tested food preparation, certain other related food preparations also tested in the same or equivalent manner (data not shown). Thus, it should be appreciated that each tested and claimed food preparation will have equivalent related preparations with demonstrated equal or equivalent reactions in the test.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

TABLE 1

| | | | |
|---|---|---|---|
| Abalone | Cured Cheese | Onion | Walnut, black |
| Adlay | Cuttlefish | Orange | Watermelon |
| Almond | Duck | Oyster | Welch Onion |
| American Cheese | Durian | Papaya | Wheat |
| Apple | Eel | Paprika | Wheat bran |
| Artichoke | Egg White (separate) | Parsley | Yeast (S. cerevisiae) |
| Asparagus | Egg Yolk (separate) | Peach | Yogurt |
| Avocado | Egg, white/yolk (comb.) | Peanut | FOOD ADDITIVES |
| Baby Bok Choy | Eggplant | Pear | Arabic Gum |
| Bamboo shoots | Garlic | Pepper, Black | Carboxymethyl Cellulose |
| Banana | Ginger | Pineapple | Carrageneenan |
| Barley, whole grain | Gluten - Gliadin | Pinto bean | FD&C Blue #1 |
| Beef | Goat's milk | Plum | FD&C Red #3 |
| Beets | Grape, white/concord | Pork | FD&C Red #40 |
| Beta-lactoglobulin | Grapefruit | Potato | FD&C Yellow #5 |
| Blueberry | Grass Carp | Rabbit | FD&C Yellow #6 |
| Broccoli | Green Onion | Rice | Gelatin |
| Buckwheat | Green pea | Roquefort Cheese | Guar Gum |
| Butter | Green pepper | Rye | Maltodextrin |
| Cabbage | Guava | Saccharine | Pectin |
| Cane sugar | Hair Tail | Safflower seed | Whey |
| Cantaloupe | Hake | Salmon | Xanthan Gum |
| Caraway | Halibut | Sardine | |
| Carrot | Hazelnut | Scallop | |
| Casein | Honey | Sesame | |
| Cashew | Kelp | Shark fin | |
| Cauliflower | Kidney bean | Sheep's milk | |
| Celery | Kiwi Fruit | Shrimp | |
| Chard | Lamb | Sole | |
| Cheddar Cheese | Leek | Soybean | |
| Chick Peas | Lemon | Spinach | |
| Chicken | Lentils | Squashes | |
| Chili pepper | Lettuce, Iceberg | Squid | |
| Chocolate | Lima bean | Strawberry | |
| Cinnamon | Lobster | String bean | |
| Clam | Longan | Sunflower seed | |
| Cocoa Bean | Mackerel | Sweet potato | |
| Coconut | Malt | Swiss cheese | |
| Codfish | Mango | Taro | |
| Coffee | Marjoram | Tea, black | |
| Cola nut | Millet | Tobacco | |
| Corn | Mung bean | Tomato | |
| Cottage cheese | Mushroom | Trout | |
| Cow's milk | Mustard seed | Tuna | |
| Crab | Oat | Turkey | |
| Cucumber | Olive | Vanilla | |

TABLE 2
Ranking of Foods according to 2-tailed Permutation
T-test p-values with FDR adjustment

| | | Raw | FDR Multiplicity- |
|---|---|---|---|
| Rank | Food | p-value | adj p-value |
| 1 | Almond | 0.0000 | 0.0000 |
| 2 | Apple | 0.0000 | 0.0000 |
| 3 | Avocado | 0.0000 | 0.0000 |
| 4 | Barley | 0.0000 | 0.0000 |
| 5 | Broccoli | 0.0000 | 0.0000 |
| 6 | Buck_Wheat | 0.0000 | 0.0000 |
| 7 | Cabbage | 0.0000 | 0.0000 |
| 8 | Cane_Sugar | 0.0000 | 0.0000 |
| 9 | Cantaloupe | 0.0000 | 0.0000 |
| 10 | Carrot | 0.0000 | 0.0000 |
| 11 | Cauliflower | 0.0000 | 0.0000 |
| 12 | Celery | 0.0000 | 0.0000 |
| 13 | Chili_Pepper | 0.0000 | 0.0000 |
| 14 | Chocolate | 0.0000 | 0.0000 |
| 15 | Clam | 0.0000 | 0.0000 |
| 16 | Cola_Nut | 0.0000 | 0.0000 |
| 17 | Corn | 0.0000 | 0.0000 |
| 18 | Cucumber | 0.0000 | 0.0000 |
| 19 | Eggplant | 0.0000 | 0.0000 |
| 20 | Garlic | 0.0000 | 0.0000 |
| 21 | Grapefruit | 0.0000 | 0.0000 |
| 22 | Green_Pea | 0.0000 | 0.0000 |
| 23 | Green_Pepper | 0.0000 | 0.0000 |
| 24 | Honey | 0.0000 | 0.0000 |
| 25 | Lemon | 0.0000 | 0.0000 |
| 26 | Lettuce | 0.0000 | 0.0000 |
| 27 | Lima_Bean | 0.0000 | 0.0000 |
| 28 | Malt | 0.0000 | 0.0000 |
| 29 | Mustard | 0.0000 | 0.0000 |
| 30 | Oat | 0.0000 | 0.0000 |
| 31 | Olive | 0.0000 | 0.0000 |
| 32 | Onion | 0.0000 | 0.0000 |
| 33 | Orange | 0.0000 | 0.0000 |
| 34 | Oyster | 0.0000 | 0.0000 |
| 35 | Peach | 0.0000 | 0.0000 |
| 36 | Pinto_Bean | 0.0000 | 0.0000 |
| 37 | Potato | 0.0000 | 0.0000 |
| 38 | Rice | 0.0000 | 0.0000 |
| 39 | Rye | 0.0000 | 0.0000 |
| 40 | Safflower | 0.0000 | 0.0000 |
| 41 | Sardine | 0.0000 | 0.0000 |
| 42 | Scallop | 0.0000 | 0.0000 |
| 43 | Soybean | 0.0000 | 0.0000 |
| 44 | Spinach | 0.0000 | 0.0000 |
| 45 | Squashes | 0.0000 | 0.0000 |
| 46 | Strawberry | 0.0000 | 0.0000 |
| 47 | String_Bean | 0.0000 | 0.0000 |
| 48 | Sunflower_Sd | 0.0000 | 0.0000 |
| 49 | Sweet_Pot_{—} | 0.0000 | 0.0000 |
| 50 | Tea | 0.0000 | 0.0000 |
| 51 | Tobacco | 0.0000 | 0.0000 |
| 52 | Tomato | 0.0000 | 0.0000 |
| 53 | Walnut_Blk | 0.0000 | 0.0000 |
| 54 | Wheat | 0.0000 | 0.0000 |
| 55 | Yeast_Baker | 0.0000 | 0.0000 |
| 56 | Yeast_Brewer | 0.0000 | 0.0000 |
| 57 | Peanut | 0.0000 | 0.0000 |
| 58 | Pineapple | 0.0000 | 0.0000 |
| 59 | Sole | 0.0000 | 0.0001 |
| 60 | Blueberry | 0.0001 | 0.0001 |
| 61 | Grape | 0.0001 | 0.0001 |
| 62 | Chicken | 0.0003 | 0.0004 |
| 63 | Cinnamon | 0.0009 | 0.0013 |
| 64 | Turkey | 0.0012 | 0.0016 |
| 65 | Butter | 0.0017 | 0.0023 |
| 66 | Cottage_Ch_{—} | 0.0023 | 0.0032 |
| 67 | Cashew | 0.0029 | 0.0039 |
| 68 | Yogurt | 0.0036 | 0.0048 |
| 69 | Cow_Milk | 0.0037 | 0.0048 |
| 70 | Egg | 0.0045 | 0.0057 |
| 71 | Millet | 0.0067 | 0.0085 |
| 72 | Coffee | 0.0086 | 0.0108 |
| 73 | Halibut | 0.0129 | 0.0159 |
| 74 | Beef | 0.0282 | 0.0343 |
| 75 | Swiss_Ch_{—} | 0.0424 | 0.0509 |
| 76 | Lobster | 0.0455 | 0.0539 |
| 77 | Parsley | 0.0469 | 0.0548 |
| 78 | Pork | 0.0530 | 0.0610 |
| 79 | Shrimp | 0.0536 | 0.0610 |
| 80 | Cheddar_Ch_{—} | 0.0608 | 0.0684 |
| 81 | Goat_Milk | 0.0704 | 0.0783 |
| 82 | Banana | 0.0799 | 0.0877 |
| 83 | Amer_Cheese | 0.0910 | 0.0987 |
| 84 | Sesame | 0.0955 | 0.1023 |
| 85 | Crab | 0.2208 | 0.2338 |
| 86 | Mushroom | 0.3495 | 0.3658 |
| 87 | Tuna | 0.4650 | 0.4810 |
| 88 | Trout | 0.5180 | 0.5298 |
| 89 | Codfish | 0.7573 | 0.7658 |
| 90 | Salmon | 0.7671 | 0.7671 |

TABLE 3
Basic Descriptive Statistics of ELISA Score by Food
and Gender Comparing Crohn's Disease to Control

| | | | | ELISA Score | | | |
|---|---|---|---|---|---|---|---|
| Sex | Food | Diagnosis | N | Mean | SD | Min | Max |
| FEMALE | Almond | Crohns | 58 | 11.414 | 18.499 | 1.236 | 90.234 |
| | | Control | 66 | 4.034 | 2.187 | 0.100 | 13.068 |
| | | Diff (1-2) | — | 7.380 | 12.745 | — | — |
| | Amer_Cheese | Crohns | 58 | 17.738 | 20.387 | 0.899 | 105.54 |
| | | Control | 66 | 23.434 | 52.616 | 0.100 | 400.00 |
| | | Diff (1-2) | — | −5.696 | 40.855 | — | — |
| | Apple | Crohns | 58 | 7.858 | 5.919 | 1.011 | 31.172 |
| | | Control | 66 | 4.432 | 3.291 | 0.100 | 15.890 |
| | | Diff (1-2) | — | 3.426 | 4.705 | — | — |
| | Avocado | Crohns | 58 | 4.821 | 4.470 | 0.225 | 21.788 |
| | | Control | 66 | 2.930 | 2.339 | 0.100 | 14.256 |
| | | Diff (1-2) | — | 1.891 | 3.500 | — | — |
| | Banana | Crohns | 58 | 11.624 | 17.193 | 1.236 | 96.643 |
| | | Control | 66 | 8.063 | 14.962 | 0.100 | 83.654 |
| | | Diff (1-2) | — | 3.561 | 16.043 | — | — |
| | Barley | Crohns | 58 | 34.802 | 25.434 | 7.684 | 111.82 |
| | | Control | 66 | 19.090 | 12.984 | 3.026 | 64.831 |
| | | Diff (1-2) | — | 15.711 | 19.800 | — | — |
| | Beef | Crohns | 58 | 11.190 | 13.116 | 2.584 | 94.265 |
| | | Control | 66 | 10.288 | 13.960 | 3.026 | 104.76 |
| | | Diff (1-2) | — | 0.902 | 13.572 | — | — |
| | Blueberry | Crohns | 58 | 7.041 | 4.009 | 1.971 | 21.953 |
| | | Control | 66 | 5.440 | 3.773 | 0.100 | 26.772 |
| | | Diff (1-2) | — | 1.600 | 3.885 | — | — |
| | Broccoli | Crohns | 58 | 15.509 | 15.704 | 2.667 | 88.361 |
| | | Control | 66 | 6.280 | 5.292 | 0.100 | 36.378 |
| | | Diff (1-2) | — | 9.229 | 11.408 | — | — |
| | Buck_Wheat | Crohns | 58 | 15.966 | 16.986 | 2.696 | 93.463 |
| | | Control | 66 | 8.034 | 4.990 | 1.316 | 29.397 |
| | | Diff (1-2) | — | 7.932 | 12.168 | — | — |
| | Butter | Crohns | 58 | 23.583 | 23.727 | 1.910 | 103.78 |
| | | Control | 66 | 21.874 | 29.162 | 0.100 | 204.33 |
| | | Diff (1-2) | — | 1.710 | 26.761 | — | — |
| | Cabbage | Crohns | 58 | 16.197 | 21.711 | 0.449 | 128.92 |
| | | Control | 66 | 7.362 | 10.123 | 0.100 | 56.932 |
| | | Diff (1-2) | — | 8.834 | 16.578 | — | — |
| | Cane_Sugar | Crohns | 58 | 42.344 | 24.843 | 8.794 | 120.18 |
| | | Control | 66 | 18.288 | 9.172 | 2.632 | 43.466 |
| | | Diff (1-2) | — | 24.056 | 18.253 | — | — |
| | Cantaloupe | Crohns | 58 | 17.507 | 19.360 | 1.011 | 100.55 |
| | | Control | 66 | 6.154 | 6.160 | 0.100 | 48.752 |
| | | Diff (1-2) | — | 11.353 | 13.977 | — | — |
| | Carrot | Crohns | 58 | 9.812 | 9.209 | 0.674 | 44.652 |
| | | Control | 66 | 4.813 | 3.705 | 0.100 | 24.141 |
| | | Diff (1-2) | — | 4.998 | 6.851 | — | — |
| | Cashew | Crohns | 58 | 13.184 | 16.448 | 1.405 | 80.692 |
| | | Control | 66 | 9.924 | 16.382 | 0.100 | 94.907 |
| | | Diff (1-2) | — | 3.260 | 16.413 | — | — |
| | Cauliflower | Crohns | 58 | 12.566 | 17.316 | 1.685 | 93.058 |
| | | Control | 66 | 5.977 | 8.336 | 0.100 | 58.808 |
| | | Diff (1-2) | — | 6.588 | 13.309 | — | — |
| | Celery | Crohns | 58 | 18.593 | 16.602 | 2.359 | 90.905 |
| | | Control | 66 | 9.634 | 5.975 | 0.395 | 32.141 |
| | | Diff (1-2) | — | 8.959 | 12.157 | — | — |
| | Cheddar_Ch_{—} | Crohns | 58 | 19.798 | 21.711 | 0.674 | 87.567 |
| | | Control | 66 | 26.852 | 55.697 | 0.100 | 400.00 |
| | | Diff (1-2) | — | −7.054 | 43.278 | — | — |
| | Chicken | Crohns | 58 | 22.202 | 13.096 | 5.864 | 70.295 |
| | | Control | 66 | 18.303 | 10.514 | 4.743 | 61.887 |
| | | Diff (1-2) | — | 3.899 | 11.791 | — | — |
| | Chili_Pepper | Crohns | 58 | 17.935 | 20.096 | 2.815 | 98.081 |
| | | Control | 66 | 8.577 | 7.784 | 0.100 | 42.583 |
| | | Diff (1-2) | — | 9.359 | 14.865 | — | — |
| | Chocolate | Crohns | 58 | 26.657 | 16.486 | 7.637 | 74.691 |
| | | Control | 66 | 14.350 | 6.578 | 3.006 | 35.317 |
| | | Diff (1-2) | — | 12.307 | 12.249 | — | — |
| | Cinnamon | Crohns | 58 | 43.483 | 30.988 | 4.494 | 176.02 |
| | | Control | 66 | 32.170 | 24.180 | 5.374 | 132.49 |
| | | Diff (1-2) | — | 11.314 | 27.571 | — | — |
| | Clam | Crohns | 58 | 68.044 | 57.734 | 9.622 | 400.00 |
| | | Control | 66 | 52.166 | 58.253 | 7.819 | 400.00 |
| | | Diff (1-2) | — | 15.878 | 58.011 | — | — |
| | Codfish | Crohns | 58 | 26.268 | 27.674 | 3.932 | 165.78 |
| | | Control | 66 | 29.652 | 31.720 | 6.200 | 168.28 |
| | | Diff (1-2) | — | −3.384 | 29.898 | — | — |
| | Coffee | Crohns | 58 | 38.597 | 61.691 | 3.815 | 333.28 |
| | | Control | 66 | 29.631 | 46.880 | 5.215 | 346.81 |
| | | Diff (1-2) | — | 8.966 | 54.305 | — | — |
| | Cola_Nut | Crohns | 58 | 40.632 | 20.269 | 14.168 | 132.60 |
| | | Control | 66 | 29.138 | 12.588 | 8.723 | 58.129 |
| | | Diff (1-2) | — | 11.494 | 16.624 | — | — |
| | Corn | Crohns | 58 | 46.036 | 64.842 | 2.022 | 289.00 |
| | | Control | 66 | 11.407 | 23.137 | 0.100 | 187.68 |
| | | Diff (1-2) | — | 34.628 | 47.430 | — | — |
| | Cottage_Ch_{—} | Crohns | 58 | 80.159 | 99.443 | 4.530 | 400.00 |
| | | Control | 66 | 76.158 | 92.333 | 0.100 | 400.00 |
| | | Diff (1-2) | — | 4.002 | 95.721 | — | — |
| | Cow_Milk | Crohns | 58 | 78.912 | 98.984 | 2.179 | 400.00 |
| | | Control | 66 | 75.882 | 86.959 | 0.100 | 400.00 |
| | | Diff (1-2) | — | 3.030 | 92.772 | — | — |
| | Crab | Crohns | 58 | 32.848 | 56.589 | 4.831 | 400.00 |
| | | Control | 66 | 23.583 | 17.654 | 3.803 | 93.236 |
| | | Diff (1-2) | — | 9.266 | 40.770 | — | — |
| | Cucumber | Crohns | 58 | 25.168 | 23.609 | 1.123 | 114.91 |
| | | Control | 66 | 8.461 | 8.149 | 0.100 | 38.939 |
| | | Diff (1-2) | — | 16.708 | 17.199 | — | — |
| | Egg | Crohns | 58 | 62.358 | 78.126 | 0.225 | 397.18 |
| | | Control | 66 | 55.102 | 89.966 | 0.100 | 400.00 |
| | | Diff (1-2) | — | 7.257 | 84.640 | — | — |
| | Eggplant | Crohns | 58 | 13.760 | 12.767 | 0.786 | 62.017 |
| | | Control | 66 | 5.732 | 5.993 | 0.100 | 31.330 |
| | | Diff (1-2) | — | 8.027 | 9.762 | — | — |
| | Garlic | Crohns | 58 | 27.792 | 21.477 | 4.382 | 90.966 |
| | | Control | 66 | 11.174 | 5.779 | 3.380 | 28.482 |
| | | Diff (1-2) | — | 16.617 | 15.274 | — | — |
| | Goat_Milk | Crohns | 58 | 13.060 | 16.554 | 0.112 | 93.821 |
| | | Control | 66 | 15.413 | 28.452 | 0.100 | 180.08 |
| | | Diff (1-2) | — | −2.353 | 23.650 | — | — |
| | Grape | Crohns | 58 | 25.633 | 16.200 | 7.623 | 96.989 |
| | | Control | 66 | 20.276 | 6.827 | 10.650 | 47.817 |
| | | Diff (1-2) | — | 5.358 | 12.143 | — | — |
| | Grapefruit | Crohns | 58 | 9.534 | 14.318 | 0.337 | 81.588 |
| | | Control | 66 | 3.278 | 2.446 | 0.100 | 14.364 |
| | | Diff (1-2) | — | 6.256 | 9.948 | — | — |
| | Green_Pea | Crohns | 58 | 25.898 | 21.338 | 1.236 | 93.790 |
| | | Control | 66 | 8.631 | 7.160 | 0.496 | 32.502 |
| | | Diff (1-2) | — | 17.267 | 15.493 | — | — |
| | Green_Pepper | Crohns | 58 | 12.633 | 17.165 | 0.674 | 94.004 |
| | | Control | 66 | 4.149 | 2.875 | 0.100 | 14.364 |
| | | Diff (1-2) | — | 8.484 | 11.919 | — | — |
| | Halibut | Crohns | 58 | 18.449 | 24.993 | 2.584 | 150.08 |
| | | Control | 66 | 11.119 | 7.129 | 2.729 | 44.884 |
| | | Diff (1-2) | — | 7.330 | 17.858 | — | — |
| | Honey | Crohns | 58 | 17.863 | 9.464 | 3.932 | 45.286 |
| | | Control | 66 | 10.185 | 4.203 | 4.227 | 19.876 |
| | | Diff (1-2) | — | 7.678 | 7.160 | — | — |
| | Lemon | Crohns | 58 | 4.934 | 4.420 | 0.112 | 23.142 |
| | | Control | 66 | 2.482 | 2.159 | 0.100 | 14.688 |
| | | Diff (1-2) | — | 2.452 | 3.407 | — | — |
| | Lettuce | Crohns | 58 | 20.793 | 20.627 | 2.696 | 92.059 |
| | | Control | 66 | 11.368 | 6.472 | 0.921 | 29.851 |
| | | Diff (1-2) | — | 9.425 | 14.870 | — | — |
| | Lima Bean | Crohns | 58 | 14.117 | 13.470 | 1.460 | 78.927 |
| | | Control | 66 | 6.624 | 8.761 | 0.100 | 65.634 |
| | | Diff (1-2) | — | 7.493 | 11.210 | — | — |
| | Lobster | Crohns | 58 | 23.321 | 51.681 | 4.831 | 400.00 |
| | | Control | 66 | 13.398 | 8.359 | 3.938 | 46.560 |
| | | Diff (1-2) | — | 9.922 | 35.849 | — | — |
| | Malt | Crohns | 58 | 30.370 | 15.705 | 9.125 | 76.468 |
| | | Control | 66 | 21.743 | 11.326 | 3.684 | 57.151 |
| | | Diff (1-2) | — | 8.627 | 13.549 | — | — |
| | Millet | Crohns | 58 | 5.256 | 2.978 | 0.899 | 15.741 |
| | | Control | 66 | 4.889 | 7.091 | 0.100 | 46.663 |
| | | Diff (1-2) | — | 0.367 | 5.562 | — | — |
| | Mushroom | Crohns | 58 | 13.830 | 15.920 | 1.891 | 88.006 |
| | | Control | 66 | 13.174 | 12.549 | 1.117 | 49.656 |
| | | Diff (1-2) | — | 0.656 | 14.224 | — | — |
| | Mustard | Crohns | 58 | 17.318 | 16.612 | 3.050 | 96.989 |
| | | Control | 66 | 8.842 | 5.224 | 0.100 | 23.452 |
| | | Diff (1-2) | — | 8.476 | 11.978 | — | — |
| | Oat | Crohns | 58 | 53.104 | 37.632 | 3.662 | 156.14 |
| | | Control | 66 | 16.237 | 14.506 | 0.100 | 76.165 |
| | | Diff (1-2) | — | 36.867 | 27.816 | — | — |
| | Olive | Crohns | 58 | 44.340 | 41.643 | 7.740 | 203.38 |
| | | Control | 66 | 23.704 | 14.281 | 5.272 | 59.488 |
| | | Diff (1-2) | — | 20.636 | 30.313 | — | — |
| | Onion | Crohns | 58 | 34.303 | 46.106 | 2.134 | 325.23 |
| | | Control | 66 | 11.329 | 16.935 | 1.184 | 114.37 |
| | | Diff (1-2) | — | 22.973 | 33.852 | — | — |
| | Orange | Crohns | 58 | 56.646 | 55.436 | 5.934 | 320.01 |
| | | Control | 66 | 15.289 | 11.608 | 1.489 | 47.125 |
| | | Diff (1-2) | — | 41.356 | 38.828 | — | — |
| | Oyster | Crohns | 58 | 90.522 | 100.157 | 11.256 | 400.00 |
| | | Control | 66 | 42.674 | 33.485 | 5.656 | 168.59 |
| | | Diff (1-2) | — | 47.848 | 72.692 | — | — |
| | Parsley | Crohns | 58 | 8.252 | 15.254 | 1.011 | 96.373 |
| | | Control | 66 | 5.005 | 6.541 | 0.100 | 34.932 |
| | | Diff (1-2) | — | 3.247 | 11.468 | — | — |
| | Peach | Crohns | 58 | 54.845 | 90.153 | 2.022 | 400.00 |
| | | Control | 66 | 7.145 | 7.742 | 0.100 | 33.820 |
| | | Diff (1-2) | — | 47.700 | 61.881 | — | — |
| | Peanut | Crohns | 58 | 8.647 | 11.328 | 1.522 | 54.418 |
| | | Control | 66 | 5.563 | 4.941 | 0.100 | 26.567 |
| | | Diff (1-2) | — | 3.084 | 8.542 | — | — |
| | Pineapple | Crohns | 58 | 49.801 | 51.537 | 2.359 | 237.27 |
| | | Control | 66 | 23.710 | 46.114 | 0.100 | 278.44 |
| | | Diff (1-2) | — | 26.092 | 48.723 | — | — |
| | Pinto_Bean | Crohns | 58 | 22.566 | 26.899 | 1.573 | 142.91 |
| | | Control | 66 | 10.138 | 8.167 | 0.100 | 48.623 |
| | | Diff (1-2) | — | 12.428 | 19.328 | — | — |
| | Pork | Crohns | 58 | 11.755 | 5.998 | 3.050 | 37.673 |
| | | Control | 66 | 15.347 | 10.345 | 4.339 | 65.759 |
| | | Diff (1-2) | — | −3.592 | 8.592 | — | — |
| | Potato | Crohns | 58 | 22.508 | 22.453 | 5.160 | 126.21 |
| | | Control | 66 | 13.615 | 6.063 | 6.200 | 40.802 |
| | | Diff (1-2) | — | 8.893 | 15.972 | — | — |
| | Rice | Crohns | 58 | 42.919 | 43.195 | 7.363 | 215.30 |
| | | Control | 66 | 21.551 | 16.950 | 3.350 | 92.642 |
| | | Diff (1-2) | — | 21.367 | 32.013 | — | — |
| | Rye | Crohns | 58 | 9.310 | 6.750 | 1.837 | 31.281 |
| | | Control | 66 | 5.237 | 3.633 | 0.100 | 22.824 |
| | | Diff (1-2) | — | 4.073 | 5.322 | — | — |
| | Safflower | Crohns | 58 | 13.373 | 9.139 | 2.247 | 47.332 |
| | | Control | 66 | 8.776 | 8.189 | 1.722 | 48.833 |
| | | Diff (1-2) | — | 4.597 | 8.646 | — | — |
| | Salmon | Crohns | 58 | 9.308 | 10.206 | 1.123 | 79.957 |
| | | Control | 66 | 9.377 | 7.261 | 2.862 | 56.530 |
| | | Diff (1-2) | — | −0.069 | 8.761 | — | — |
| | Sardine | Crohns | 58 | 61.987 | 33.053 | 20.859 | 220.92 |
| | | Control | 66 | 37.084 | 16.695 | 7.190 | 88.964 |
| | | Diff (1-2) | — | 24.903 | 25.670 | — | — |
| | Scallop | Crohns | 58 | 87.917 | 47.804 | 16.309 | 237.55 |
| | | Control | 66 | 64.291 | 29.551 | 18.605 | 148.58 |
| | | Diff (1-2) | — | 23.626 | 39.153 | — | — |
| | Sesame | Crohns | 58 | 81.590 | 101.498 | 4.452 | 400.00 |
| | | Control | 66 | 80.704 | 93.902 | 5.984 | 400.00 |
| | | Diff (1-2) | — | 0.886 | 97.525 | — | — |
| | Shrimp | Crohns | 58 | 28.277 | 33.840 | 4.770 | 233.61 |
| | | Control | 66 | 33.150 | 27.875 | 6.607 | 113.66 |
| | | Diff (1-2) | — | −4.874 | 30.806 | — | — |
| | Sole | Crohns | 58 | 9.218 | 16.720 | 2.584 | 131.38 |
| | | Control | 66 | 6.440 | 6.960 | 0.100 | 54.883 |
| | | Diff (1-2) | — | 2.778 | 12.507 | — | — |
| | Soybean | Crohns | 58 | 25.942 | 27.051 | 4.926 | 149.91 |
| | | Control | 66 | 15.294 | 9.373 | 2.481 | 49.071 |
| | | Diff (1-2) | — | 10.648 | 19.716 | — | — |
| | Spinach | Crohns | 58 | 33.758 | 27.556 | 6.450 | 152.37 |
| | | Control | 66 | 20.485 | 13.172 | 6.051 | 66.626 |
| | | Diff (1-2) | — | 13.273 | 21.147 | — | — |
| | Squashes | Crohns | 58 | 20.712 | 12.860 | 4.494 | 62.663 |
| | | Control | 66 | 13.415 | 11.597 | 1.842 | 74.279 |
| | | Diff (1-2) | — | 7.298 | 12.204 | — | — |
| | Strawberry | Crohns | 58 | 9.591 | 6.255 | 1.877 | 34.746 |
| | | Control | 66 | 5.563 | 5.305 | 0.100 | 35.745 |
| | | Diff (1-2) | — | 4.028 | 5.768 | — | — |
| | String_Bean | Crohns | 58 | 78.838 | 59.978 | 21.629 | 400.00 |
| | | Control | 66 | 41.957 | 22.678 | 9.539 | 125.69 |
| | | Diff (1-2) | — | 36.881 | 44.212 | — | — |
| | Sunflower_Sd | Crohns | 58 | 19.008 | 20.344 | 2.471 | 110.48 |
| | | Control | 66 | 9.948 | 6.094 | 2.632 | 33.347 |
| | | Diff (1-2) | — | 9.060 | 14.600 | — | — |
| | Sweet_Pot | Crohns | 58 | 24.700 | 37.844 | 1.460 | 224.37 |
| | | Control | 66 | 8.592 | 4.479 | 0.395 | 25.009 |
| | | Diff (1-2) | — | 16.108 | 26.074 | — | — |
| | Swiss_Ch_{—} | Crohns | 58 | 30.278 | 39.042 | 0.899 | 182.30 |
| | | Control | 66 | 39.219 | 73.725 | 0.100 | 400.00 |
| | | Diff (1-2) | — | −8.942 | 60.067 | — | — |
| | Tea | Crohns | 58 | 46.386 | 18.239 | 14.861 | 93.341 |
| | | Control | 66 | 29.771 | 12.014 | 11.634 | 64.535 |
| | | Diff (1-2) | — | 16.615 | 15.242 | — | — |
| | Tobacco | Crohns | 58 | 65.703 | 46.048 | 19.182 | 302.94 |
| | | Control | 66 | 33.566 | 16.789 | 7.809 | 82.097 |
| | | Diff (1-2) | — | 32.137 | 33.777 | — | — |
| | Tomato | Crohns | 58 | 40.117 | 50.209 | 3.146 | 291.70 |
| | | Control | 66 | 9.066 | 7.694 | 0.100 | 42.078 |
| | | Diff (1-2) | — | 31.051 | 34.776 | — | — |
| | Trout | Crohns | 58 | 16.435 | 18.602 | 4.921 | 142.68 |
| | | Control | 66 | 16.138 | 10.667 | 5.596 | 76.221 |
| | | Diff (1-2) | — | 0.297 | 14.910 | — | — |
| | Tuna | Crohns | 58 | 15.967 | 14.389 | 4.157 | 107.15 |
| | | Control | 66 | 18.092 | 12.707 | 3.873 | 64.090 |
| | | Diff (1-2) | — | −2.125 | 13.519 | — | — |
| | Turkey | Crohns | 58 | 17.841 | 10.299 | 3.362 | 52.713 |
| | | Control | 66 | 14.461 | 6.976 | 4.094 | 32.151 |
| | | Diff (1-2) | — | 3.379 | 8.688 | — | — |
| | Walnut_Blk | Crohns | 58 | 50.033 | 52.244 | 5.843 | 306.51 |
| | | Control | 66 | 25.386 | 17.254 | 6.943 | 117.46 |
| | | Diff (1-2) | — | 24.647 | 37.866 | — | — |
| | Wheat | Crohns | 58 | 30.673 | 29.650 | 4.831 | 143.22 |
| | | Control | 66 | 18.402 | 29.364 | 0.790 | 209.95 |
| | | Diff (1-2) | — | 12.271 | 29.498 | — | — |
| | Yeast_Baker | Crohns | 58 | 31.263 | 39.826 | 2.346 | 153.39 |
| | | Control | 66 | 5.545 | 3.349 | 0.526 | 18.811 |
| | | Diff (1-2) | — | 25.718 | 27.332 | — | — |
| | Yeast_Brewer | Crohns | 58 | 76.650 | 101.592 | 3.519 | 400.00 |
| | | Control | 66 | 10.847 | 7.818 | 0.100 | 43.887 |
| | | Diff (1-2) | — | 65.803 | 69.675 | — | — |
| | Yogurt | Crohns | 58 | 22.658 | 16.068 | 5.142 | 71.316 |
| | | Control | 66 | 22.930 | 30.973 | 0.100 | 215.73 |
| | | Diff (1-2) | — | −0.272 | 25.134 | — | — |
| MALE | Almond | Crohns | 42 | 17.262 | 23.363 | 1.436 | 106.76 |
| | | Control | 97 | 4.049 | 2.231 | 0.100 | 12.591 |
| | | Diff (1-2) | — | 13.213 | 12.916 | — | — |
| | Amer_Cheese | Crohns | 42 | 58.923 | 86.967 | 1.794 | 400.00 |
| | | Control | 97 | 22.619 | 34.069 | 0.468 | 197.38 |
| | | Diff (1-2) | — | 36.304 | 55.469 | — | — |
| | Apple | Crohns | 42 | 20.657 | 56.474 | 2.034 | 370.43 |
| | | Control | 97 | 4.383 | 2.900 | 0.100 | 13.795 |
| | | Diff (1-2) | — | 16.274 | 30.990 | — | — |
| | Avocado | Crohns | 42 | 9.228 | 15.333 | 1.077 | 98.692 |
| | | Control | 97 | 2.720 | 2.992 | 0.100 | 28.693 |
| | | Diff (1-2) | — | 6.509 | 8.754 | — | — |
| | Banana | Crohns | 42 | 15.772 | 21.258 | 1.842 | 83.534 |
| | | Control | 97 | 8.576 | 36.151 | 0.100 | 350.69 |
| | | Diff (1-2) | — | 7.196 | 32.420 | — | — |
| | Barley | Crohns | 42 | 52.245 | 49.203 | 14.828 | 261.29 |
| | | Control | 97 | 19.214 | 11.923 | 4.612 | 58.865 |
| | | Diff (1-2) | — | 33.030 | 28.708 | — | — |
| | Beef | Crohns | 42 | 27.550 | 62.343 | 3.714 | 400.00 |
| | | Control | 97 | 9.327 | 11.981 | 2.059 | 93.494 |
| | | Diff (1-2) | — | 18.223 | 35.549 | — | — |
| | Blueberry | Crohns | 42 | 14.311 | 21.667 | 2.034 | 120.26 |
| | | Control | 97 | 5.393 | 2.868 | 0.100 | 19.410 |
| | | Diff (1-2) | — | 8.918 | 12.094 | — | — |
| | Broccoli | Crohns | 42 | 22.097 | 26.056 | 2.993 | 116.59 |
| | | Control | 97 | 6.790 | 8.012 | 0.131 | 72.543 |
| | | Diff (1-2) | — | 15.307 | 15.753 | — | — |
| | Buck_Wheat | Crohns | 42 | 25.016 | 25.714 | 4.067 | 120.81 |
| | | Control | 97 | 6.978 | 3.384 | 2.656 | 24.338 |
| | | Diff (1-2) | — | 18.037 | 14.349 | — | — |
| | Butter | Crohns | 42 | 50.920 | 65.643 | 6.818 | 400.00 |
| | | Control | 97 | 17.846 | 20.091 | 1.490 | 131.60 |
| | | Diff (1-2) | — | 33.074 | 39.654 | — | — |
| | Cabbage | Crohns | 42 | 31.716 | 54.498 | 1.612 | 318.14 |
| | | Control | 97 | 6.540 | 18.133 | 0.100 | 174.96 |
| | | Diff (1-2) | — | 25.175 | 33.455 | — | — |
| | Cane_Sugar | Crohns | 42 | 53.073 | 42.539 | 15.994 | 239.63 |
| | | Control | 97 | 22.356 | 18.718 | 2.789 | 100.82 |
| | | Diff (1-2) | — | 30.718 | 28.054 | — | — |
| | Cantaloupe | Crohns | 42 | 39.473 | 57.587 | 3.799 | 254.55 |
| | | Control | 97 | 6.052 | 5.569 | 0.468 | 38.706 |
| | | Diff (1-2) | — | 33.421 | 31.846 | — | — |
| | Carrot | Crohns | 42 | 20.693 | 24.226 | 2.188 | 100.85 |
| | | Control | 97 | 4.684 | 3.636 | 0.468 | 28.593 |
| | | Diff (1-2) | — | 16.009 | 13.598 | — | — |
| | Cashew | Crohns | 42 | 18.420 | 19.797 | 2.905 | 108.51 |
| | | Control | 97 | 8.362 | 10.271 | 0.100 | 55.749 |
| | | Diff (1-2) | — | 10.058 | 13.828 | — | — |
| | Cauliflower | Crohns | 42 | 24.142 | 39.843 | 1.675 | 223.18 |
| | | Control | 97 | 4.385 | 4.396 | 0.100 | 36.593 |
| | | Diff (1-2) | — | 19.757 | 22.105 | — | — |
| | Celery | Crohns | 42 | 30.174 | 34.183 | 4.489 | 169.54 |
| | | Control | 97 | 8.930 | 4.985 | 2.394 | 26.982 |
| | | Diff (1-2) | — | 21.244 | 19.160 | — | — |
| | Cheddar_Ch_{—} | Crohns | 42 | 77.938 | 106.414 | 2.273 | 400.00 |
| | | Control | 97 | 28.479 | 49.022 | 1.169 | 298.91 |
| | | Diff (1-2) | — | 49.459 | 71.224 | — | — |
| | Chicken | Crohns | 42 | 27.328 | 18.319 | 8.092 | 95.333 |
| | | Control | 97 | 17.778 | 11.456 | 5.137 | 69.503 |
| | | Diff (1-2) | — | 9.549 | 13.870 | — | — |
| | Chili_Pepper | Crohns | 42 | 28.848 | 33.455 | 2.878 | 172.60 |
| | | Control | 97 | 7.802 | 5.945 | 1.591 | 31.070 |
| | | Diff (1-2) | — | 21.047 | 18.966 | — | — |
| | Chocolate | Crohns | 42 | 35.466 | 25.625 | 10.209 | 125.20 |
| | | Control | 97 | 16.536 | 11.276 | 1.726 | 63.673 |
| | | Diff (1-2) | — | 18.930 | 16.900 | — | — |
| | Cinnamon | Crohns | 42 | 62.380 | 62.899 | 11.721 | 400.00 |
| | | Control | 97 | 35.928 | 28.520 | 3.136 | 146.95 |
| | | Diff (1-2) | — | 26.452 | 41.880 | — | — |
| | Clam | Crohns | 42 | 77.819 | 55.453 | 21.341 | 368.73 |
| | | Control | 97 | 38.293 | 21.598 | 6.370 | 103.47 |
| | | Diff (1-2) | — | 39.526 | 35.315 | — | — |
| | Codfish | Crohns | 42 | 26.808 | 16.763 | 8.829 | 83.014 |
| | | Control | 97 | 22.538 | 29.644 | 4.176 | 269.16 |
| | | Diff (1-2) | — | 4.271 | 26.455 | — | — |
| | Coffee | Crohns | 42 | 51.458 | 77.296 | 5.413 | 369.56 |
| | | Control | 97 | 20.037 | 24.002 | 2.705 | 192.24 |
| | | Diff (1-2) | — | 31.421 | 46.816 | — | — |
| | Cola_Nut | Crohns | 42 | 50.915 | 21.913 | 27.513 | 133.23 |
| | | Control | 97 | 32.919 | 20.025 | 3.851 | 112.10 |
| | | Diff (1-2) | — | 17.997 | 20.608 | — | — |
| | Corn | Crohns | 42 | 77.338 | 97.088 | 5.307 | 400.00 |
| | | Control | 97 | 10.126 | 15.048 | 1.520 | 117.90 |
| | | Diff (1-2) | — | 67.213 | 54.586 | — | — |
| | Cottage_Ch_{—} | Crohns | 42 | 182.058 | 151.988 | 8.659 | 400.00 |
| | | Control | 97 | 74.814 | 101.386 | 1.446 | 400.00 |
| | | Diff (1-2) | — | 107.244 | 118.811 | — | — |
| | Cow_Milk | Crohns | 42 | 162.668 | 142.624 | 5.957 | 400.00 |
| | | Control | 97 | 68.606 | 94.032 | 1.343 | 400.00 |
| | | Diff (1-2) | — | 94.062 | 110.831 | — | — |
| | Crab | Crohns | 42 | 26.988 | 16.382 | 6.991 | 75.776 |
| | | Control | 97 | 24.550 | 29.311 | 3.108 | 252.41 |
| | | Diff (1-2) | — | 2.438 | 26.122 | — | — |
| | Cucumber | Crohns | 42 | 52.094 | 64.653 | 3.684 | 346.20 |
| | | Control | 97 | 8.320 | 9.298 | 0.234 | 69.188 |
| | | Diff (1-2) | — | 43.774 | 36.215 | — | — |
| | Egg | Crohns | 42 | 110.719 | 122.437 | 2.533 | 400.00 |
| | | Control | 97 | 44.335 | 66.828 | 0.100 | 400.00 |
| | | Diff (1-2) | — | 66.384 | 87.268 | — | — |
| | Eggplant | Crohns | 42 | 23.965 | 27.503 | 1.612 | 136.32 |
| | | Control | 97 | 5.856 | 10.455 | 0.100 | 92.376 |
| | | Diff (1-2) | — | 18.109 | 17.406 | — | — |
| | Garlic | Crohns | 42 | 39.211 | 57.002 | 3.110 | 336.25 |
| | | Control | 97 | 13.476 | 12.122 | 3.097 | 70.591 |
| | | Diff (1-2) | — | 25.736 | 32.793 | — | — |
| | Goat_Milk | Crohns | 42 | 46.468 | 68.485 | 1.914 | 400.00 |
| | | Control | 97 | 17.999 | 36.202 | 0.100 | 275.19 |
| | | Diff (1-2) | — | 28.469 | 48.187 | — | — |
| | Grape | Crohns | 42 | 35.644 | 19.334 | 8.253 | 98.030 |
| | | Control | 97 | 23.308 | 7.422 | 11.900 | 41.654 |
| | | Diff (1-2) | — | 12.336 | 12.267 | — | — |
| | Grapefruit | Crohns | 42 | 21.288 | 42.785 | 1.077 | 254.55 |
| | | Control | 97 | 3.049 | 2.306 | 0.100 | 14.648 |
| | | Diff (1-2) | — | 18.239 | 23.485 | — | — |
| | Green_Pea | Crohns | 42 | 42.880 | 42.302 | 4.144 | 195.47 |
| | | Control | 97 | 9.229 | 11.366 | 0.100 | 71.765 |
| | | Diff (1-2) | — | 33.651 | 25.021 | — | — |
| | Green_Pepper | Crohns | 42 | 22.243 | 27.678 | 1.957 | 125.37 |
| | | Control | 97 | 3.972 | 2.664 | 0.100 | 15.744 |
| | | Diff (1-2) | — | 18.271 | 15.305 | — | — |
| | Halibut | Crohns | 42 | 15.927 | 6.826 | 6.404 | 37.687 |
| | | Control | 97 | 12.657 | 15.451 | 0.818 | 142.09 |
| | | Diff (1-2) | — | 3.270 | 13.462 | — | — |
| | Honey | Crohns | 42 | 33.216 | 51.794 | 6.220 | 311.65 |
| | | Control | 97 | 11.082 | 6.215 | 2.434 | 31.202 |
| | | Diff (1-2) | — | 22.133 | 28.808 | — | — |
| | Lemon | Crohns | 42 | 8.874 | 11.301 | 1.077 | 68.148 |
| | | Control | 97 | 2.310 | 1.436 | 0.100 | 8.383 |
| | | Diff (1-2) | — | 6.564 | 6.298 | — | — |
| | Lettuce | Crohns | 42 | 26.717 | 22.581 | 4.905 | 111.56 |
| | | Control | 97 | 11.271 | 8.295 | 2.871 | 52.209 |
| | | Diff (1-2) | — | 15.446 | 14.171 | — | — |
| | Lima Bean | Crohns | 42 | 22.657 | 32.002 | 2.034 | 205.58 |
| | | Control | 97 | 5.994 | 5.650 | 0.100 | 37.640 |
| | | Diff (1-2) | — | 16.663 | 18.135 | — | — |
| | Lobster | Crohns | 42 | 21.549 | 27.138 | 4.834 | 155.05 |
| | | Control | 97 | 15.678 | 11.555 | 0.468 | 61.064 |
| | | Diff (1-2) | — | 5.871 | 17.719 | — | — |
| | Malt | Crohns | 42 | 41.328 | 29.793 | 11.178 | 155.10 |
| | | Control | 97 | 21.137 | 12.373 | 3.182 | 58.638 |
| | | Diff (1-2) | — | 20.191 | 19.311 | — | — |
| | Millet | Crohns | 42 | 7.941 | 7.520 | 1.914 | 50.638 |
| | | Control | 97 | 4.006 | 6.783 | 0.100 | 67.831 |
| | | Diff (1-2) | — | 3.935 | 7.011 | — | — |
| | Mushroom | Crohns | 42 | 15.893 | 14.335 | 2.695 | 67.757 |
| | | Control | 97 | 12.883 | 12.397 | 1.350 | 59.949 |
| | | Diff (1-2) | — | 3.011 | 13.007 | — | — |
| | Mustard | Crohns | 42 | 28.936 | 23.513 | 2.512 | 119.29 |
| | | Control | 97 | 9.168 | 5.413 | 1.044 | 28.538 |
| | | Diff (1-2) | — | 19.768 | 13.638 | — | — |
| | Oat | Crohns | 42 | 88.964 | 100.453 | 6.190 | 400.00 |
| | | Control | 97 | 20.964 | 22.946 | 1.461 | 107.25 |
| | | Diff (1-2) | — | 68.000 | 58.214 | — | — |
| | Olive | Crohns | 42 | 75.419 | 79.624 | 9.569 | 400.00 |
| | | Control | 97 | 24.794 | 22.708 | 5.137 | 160.63 |
| | | Diff (1-2) | — | 50.624 | 47.526 | — | — |
| | Onion | Crohns | 42 | 64.267 | 95.713 | 5.519 | 400.00 |
| | | Control | 97 | 11.600 | 17.551 | 1.175 | 158.57 |
| | | Diff (1-2) | — | 52.668 | 54.383 | — | — |
| | Orange | Crohns | 42 | 104.865 | 123.756 | 10.406 | 400.00 |
| | | Control | 97 | 17.767 | 16.361 | 2.146 | 79.419 |
| | | Diff (1-2) | — | 87.099 | 69.073 | — | — |
| | Oyster | Crohns | 42 | 99.339 | 73.045 | 11.003 | 400.00 |
| | | Control | 97 | 43.016 | 35.689 | 5.069 | 216.58 |
| | | Diff (1-2) | — | 56.322 | 49.893 | — | — |
| | Parsley | Crohns | 42 | 6.736 | 6.342 | 0.957 | 40.451 |
| | | Control | 97 | 4.867 | 7.352 | 0.100 | 58.674 |
| | | Diff (1-2) | — | 1.869 | 7.064 | — | — |
| | Peach | Crohns | 42 | 94.609 | 125.202 | 2.533 | 400.00 |
| | | Control | 97 | 8.390 | 8.373 | 0.100 | 50.444 |
| | | Diff (1-2) | — | 86.218 | 68.850 | — | — |
| | Peanut | Crohns | 42 | 13.239 | 13.788 | 2.122 | 53.403 |
| | | Control | 97 | 4.241 | 4.514 | 0.855 | 41.070 |
| | | Diff (1-2) | — | 8.998 | 8.436 | — | — |
| | Pineapple | Crohns | 42 | 62.940 | 75.107 | 2.871 | 290.38 |
| | | Control | 97 | 23.259 | 48.769 | 0.100 | 400.00 |
| | | Diff (1-2) | — | 39.681 | 57.921 | — | — |
| | Pinto_Bean | Crohns | 42 | 45.081 | 65.153 | 2.512 | 276.95 |
| | | Control | 97 | 8.132 | 5.524 | 0.664 | 28.288 |
| | | Diff (1-2) | — | 36.949 | 35.941 | — | — |
| | Pork | Crohns | 42 | 17.840 | 12.584 | 4.673 | 59.737 |
| | | Control | 97 | 13.403 | 10.218 | 1.637 | 57.274 |
| | | Diff (1-2) | — | 4.437 | 10.980 | — | — |
| | Potato | Crohns | 42 | 46.223 | 54.338 | 7.331 | 238.36 |
| | | Control | 97 | 14.555 | 5.951 | 5.259 | 49.002 |
| | | Diff (1-2) | — | 31.668 | 30.140 | — | — |
| | Rice | Crohns | 42 | 79.096 | 80.923 | 5.981 | 400.00 |
| | | Control | 97 | 25.220 | 18.948 | 5.149 | 118.12 |
| | | Diff (1-2) | — | 53.876 | 47.025 | — | — |
| | Rye | Crohns | 42 | 16.215 | 14.726 | 1.794 | 64.767 |
| | | Control | 97 | 4.801 | 2.690 | 0.653 | 15.288 |
| | | Diff (1-2) | — | 11.414 | 8.365 | — | — |
| | Safflower | Crohns | 42 | 26.206 | 23.147 | 3.230 | 91.530 |
| | | Control | 97 | 8.672 | 6.177 | 1.958 | 38.914 |
| | | Diff (1-2) | — | 17.534 | 13.678 | — | — |
| | Salmon | Crohns | 42 | 12.739 | 12.048 | 2.695 | 60.685 |
| | | Control | 97 | 10.920 | 13.350 | 0.100 | 125.74 |
| | | Diff (1-2) | — | 1.818 | 12.974 | — | — |
| | Sardine | Crohns | 42 | 78.052 | 43.740 | 23.170 | 235.45 |
| | | Control | 97 | 37.035 | 15.979 | 7.037 | 90.406 |
| | | Diff (1-2) | — | 41.017 | 27.413 | — | — |
| | Scallop | Crohns | 42 | 95.485 | 59.343 | 19.062 | 284.23 |
| | | Control | 97 | 60.721 | 32.618 | 8.942 | 167.75 |
| | | Diff (1-2) | — | 34.764 | 42.420 | — | — |
| | Sesame | Crohns | 42 | 103.488 | 125.523 | 1.675 | 400.00 |
| | | Control | 97 | 60.406 | 79.861 | 2.115 | 400.00 |
| | | Diff (1-2) | — | 43.082 | 95.835 | — | — |
| | Shrimp | Crohns | 42 | 22.964 | 18.934 | 4.943 | 90.318 |
| | | Control | 97 | 34.490 | 42.689 | 2.663 | 342.67 |
| | | Diff (1-2) | — | −11.526 | 37.205 | — | — |
| | Sole | Crohns | 42 | 10.212 | 4.988 | 4.604 | 34.993 |
| | | Control | 97 | 4.912 | 2.238 | 0.100 | 14.303 |
| | | Diff (1-2) | — | 5.300 | 3.310 | — | — |
| | Soybean | Crohns | 42 | 75.898 | 120.882 | 5.144 | 400.00 |
| | | Control | 97 | 15.880 | 9.273 | 4.912 | 71.264 |
| | | Diff (1-2) | — | 60.018 | 66.583 | — | — |
| | Spinach | Crohns | 42 | 60.138 | 65.262 | 4.785 | 358.33 |
| | | Control | 97 | 14.656 | 7.304 | 3.054 | 39.867 |
| | | Diff (1-2) | — | 45.482 | 36.222 | — | — |
| | Squashes | Crohns | 42 | 28.999 | 20.712 | 5.168 | 88.662 |
| | | Control | 97 | 12.688 | 7.539 | 1.637 | 49.775 |
| | | Diff (1-2) | — | 16.311 | 12.970 | — | — |
| | Strawberry | Crohns | 42 | 26.245 | 65.451 | 1.794 | 400.00 |
| | | Control | 97 | 4.767 | 4.446 | 0.100 | 30.664 |
| | | Diff (1-2) | — | 21.478 | 35.998 | — | — |
| | String_Bean | Crohns | 42 | 112.366 | 85.891 | 31.810 | 400.00 |
| | | Control | 97 | 40.720 | 22.088 | 5.609 | 141.76 |
| | | Diff (1-2) | — | 71.646 | 50.494 | — | — |
| | Sunflower_Sd | Crohns | 42 | 29.361 | 28.120 | 3.708 | 142.57 |
| | | Control | 97 | 9.071 | 5.842 | 2.523 | 46.948 |
| | | Diff (1-2) | — | 20.290 | 16.142 | — | — |
| | Sweet_Pot | Crohns | 42 | 33.068 | 42.788 | 3.708 | 219.80 |
| | | Control | 97 | 8.456 | 4.878 | 0.100 | 30.052 |
| | | Diff (1-2) | — | 24.611 | 23.761 | — | — |
| | Swiss_Ch_{—} | Crohns | 42 | 113.961 | 131.768 | 2.034 | 400.00 |
| | | Control | 97 | 43.413 | 79.791 | 0.100 | 400.00 |
| | | Diff (1-2) | — | 70.547 | 98.272 | — | — |
| | Tea | Crohns | 42 | 64.359 | 37.277 | 25.093 | 223.18 |
| | | Control | 97 | 31.353 | 13.716 | 8.890 | 70.271 |
| | | Diff (1-2) | — | 33.006 | 23.403 | — | — |
| | Tobacco | Crohns | 42 | 89.634 | 59.808 | 18.199 | 280.05 |
| | | Control | 97 | 39.354 | 26.787 | 6.106 | 134.30 |
| | | Diff (1-2) | — | 50.280 | 39.665 | — | — |
| | Tomato | Crohns | 42 | 78.851 | 104.229 | 4.828 | 400.00 |
| | | Control | 97 | 9.088 | 7.957 | 0.100 | 48.338 |
| | | Diff (1-2) | — | 69.763 | 57.407 | — | — |
| | Trout | Crohns | 42 | 20.187 | 17.827 | 5.730 | 101.47 |
| | | Control | 97 | 16.891 | 15.673 | 0.100 | 144.46 |
| | | Diff (1-2) | — | 3.297 | 16.347 | — | — |
| | Tuna | Crohns | 42 | 18.234 | 13.441 | 5.617 | 64.332 |
| | | Control | 97 | 18.392 | 16.755 | 3.156 | 110.69 |
| | | Diff (1-2) | — | −0.158 | 15.836 | — | — |
| | Turkey | Crohns | 42 | 20.817 | 11.269 | 5.742 | 55.914 |
| | | Control | 97 | 14.840 | 10.829 | 2.789 | 69.572 |
| | | Diff (1-2) | — | 5.977 | 10.963 | — | — |
| | Walnut_Blk | Crohns | 42 | 80.734 | 94.320 | 5.622 | 400.00 |
| | | Control | 97 | 25.520 | 14.492 | 4.249 | 71.927 |
| | | Diff (1-2) | — | 55.213 | 53.005 | — | — |
| | Wheat | Crohns | 42 | 61.572 | 76.994 | 5.742 | 400.00 |
| | | Control | 97 | 14.494 | 12.413 | 2.741 | 90.037 |
| | | Diff (1-2) | — | 47.078 | 43.383 | — | — |
| | Yeast_Baker | Crohns | 42 | 53.229 | 90.889 | 3.946 | 400.00 |
| | | Control | 97 | 9.617 | 17.250 | 1.305 | 116.43 |
| | | Diff (1-2) | — | 43.612 | 51.776 | — | — |
| | Yeast_Brewer | Crohns | 42 | 95.893 | 127.082 | 4.964 | 400.00 |
| | | Control | 97 | 22.646 | 47.630 | 1.931 | 308.34 |
| | | Diff (1-2) | — | 73.248 | 80.143 | — | — |
| | Yogurt | Crohns | 42 | 50.857 | 64.275 | 5.981 | 400.00 |
| | | Control | 97 | 19.210 | 20.751 | 0.234 | 120.51 |
| | | Diff (1-2) | — | 31.646 | 39.219 | — | — |

TABLE 4
Upper Quantiles of ELISA Signal Scores among Control Subjects
as Candidates for Test Cutpoints in Determining “Positive”
or “Negative” Top 83 Foods Ranked by Descending
order of Discriminatory Ability using Permutation Test
Crohn's Subjects vs. Controls

| Food | | | Cutpoint | |
|---|---|---|---|---|
| Ranking | Food | Sex | 90th percentile | 95th percentile |
| 1 | Almond | FEMALE | 6.784 | 8.230 |
| | | MALE | 7.220 | 8.752 |
| 2 | Apple | FEMALE | 9.112 | 11.832 |
| | | MALE | 8.574 | 10.526 |
| 3 | Avocado | FEMALE | 5.445 | 7.256 |
| | | MALE | 4.450 | 5.544 |
| 4 | Barley | FEMALE | 35.074 | 46.987 |
| | | MALE | 36.226 | 45.783 |
| 5 | Broccoli | FEMALE | 11.868 | 14.788 |
| | | MALE | 13.164 | 16.081 |
| 6 | Buck_Wheat | FEMALE | 14.821 | 18.522 |
| | | MALE | 11.366 | 12.764 |
| 7 | Cabbage | FEMALE | 18.329 | 28.855 |
| | | MALE | 9.780 | 18.430 |
| 8 | Cane_Sugar | FEMALE | 29.845 | 36.257 |
| | | MALE | 45.879 | 65.784 |
| 9 | Cantaloupe | FEMALE | 9.668 | 13.791 |
| | | MALE | 11.366 | 16.211 |
| 10 | Carrot | FEMALE | 9.210 | 11.335 |
| | | MALE | 7.709 | 10.652 |
| 11 | Cauliflower | FEMALE | 11.601 | 17.389 |
| | | MALE | 7.934 | 11.071 |
| 12 | Celery | FEMALE | 17.153 | 22.370 |
| | | MALE | 15.081 | 19.641 |
| 13 | Chili_Pepper | FEMALE | 16.351 | 25.034 |
| | | MALE | 13.873 | 21.294 |
| 14 | Chocolate | FEMALE | 23.547 | 25.870 |
| | | MALE | 32.778 | 38.001 |
| 15 | Clam | FEMALE | 98.048 | 157.97 |
| | | MALE | 66.421 | 78.340 |
| 16 | Cola_Nut | FEMALE | 48.364 | 53.590 |
| | | MALE | 60.115 | 72.797 |
| 17 | Corn | FEMALE | 19.964 | 31.012 |
| | | MALE | 19.652 | 29.904 |
| 18 | Cucumber | FEMALE | 20.943 | 26.865 |
| | | MALE | 17.834 | 23.952 |
| 19 | Eggplant | FEMALE | 12.669 | 18.880 |
| | | MALE | 9.335 | 14.470 |
| 20 | Garlic | FEMALE | 19.404 | 22.718 |
| | | MALE | 27.466 | 41.576 |
| 21 | Grapefruit | FEMALE | 6.228 | 7.631 |
| | | MALE | 5.286 | 7.613 |
| 22 | Green_Pea | FEMALE | 20.747 | 23.644 |
| | | MALE | 19.683 | 32.336 |
| 23 | Green_Pepper | FEMALE | 8.323 | 10.363 |
| | | MALE | 6.961 | 9.614 |
| 24 | Honey | FEMALE | 16.290 | 17.436 |
| | | MALE | 19.283 | 24.990 |
| 25 | Lemon | FEMALE | 4.582 | 5.956 |
| | | MALE | 4.132 | 5.172 |
| 26 | Lettuce | FEMALE | 20.526 | 24.133 |
| | | MALE | 18.497 | 28.530 |
| 27 | Lima_Bean | FEMALE | 12.681 | 18.987 |
| | | MALE | 10.695 | 14.574 |
| 28 | Malt | FEMALE | 36.583 | 41.718 |
| | | MALE | 39.324 | 45.906 |
| 29 | Mustard | FEMALE | 17.495 | 19.371 |
| | | MALE | 16.207 | 20.950 |
| 30 | Oat | FEMALE | 33.287 | 44.796 |
| | | MALE | 55.429 | 73.538 |
| 31 | Olive | FEMALE | 48.147 | 55.209 |
| | | MALE | 42.414 | 60.363 |
| 32 | Onion | FEMALE | 20.739 | 37.607 |
| | | MALE | 25.532 | 33.348 |
| 33 | Orange | FEMALE | 33.733 | 40.684 |
| | | MALE | 36.963 | 56.348 |
| 34 | Oyster | FEMALE | 85.694 | 114.99 |
| | | MALE | 82.753 | 119.27 |
| 35 | Peach | FEMALE | 18.124 | 26.741 |
| | | MALE | 17.565 | 26.495 |
| 36 | Pinto_Bean | FEMALE | 18.971 | 27.653 |
| | | MALE | 16.002 | 20.472 |
| 37 | Potato | FEMALE | 20.119 | 25.130 |
| | | MALE | 21.094 | 24.115 |
| 38 | Rice | FEMALE | 40.517 | 58.645 |
| | | MALE | 51.781 | 63.091 |
| 39 | Rye | FEMALE | 8.541 | 12.208 |
| | | MALE | 8.375 | 10.663 |
| 40 | Safflower | FEMALE | 16.119 | 24.720 |
| | | MALE | 16.213 | 21.375 |
| 41 | Sardine | FEMALE | 58.859 | 73.780 |
| | | MALE | 57.306 | 64.787 |
| 42 | Scallop | FEMALE | 103.91 | 117.22 |
| | | MALE | 108.83 | 127.84 |
| 43 | Soybean | FEMALE | 30.747 | 34.594 |
| | | MALE | 26.296 | 31.259 |
| 44 | Spinach | FEMALE | 38.040 | 48.124 |
| | | MALE | 24.903 | 28.543 |
| 45 | Squashes | FEMALE | 22.106 | 32.802 |
| | | MALE | 22.798 | 25.920 |
| 46 | Strawberry | FEMALE | 10.404 | 15.163 |
| | | MALE | 8.880 | 13.628 |
| 47 | String_Bean | FEMALE | 68.820 | 84.595 |
| | | MALE | 65.416 | 83.772 |
| 48 | Sunflower_Sd | FEMALE | 16.586 | 22.668 |
| | | MALE | 14.229 | 18.509 |
| 49 | Sweet_Pot_{—} | FEMALE | 14.612 | 17.269 |
| | | MALE | 13.809 | 18.111 |
| 50 | Tea | FEMALE | 46.190 | 53.329 |
| | | MALE | 49.935 | 56.719 |
| 51 | Tobacco | FEMALE | 57.851 | 64.450 |
| | | MALE | 74.551 | 102.34 |
| 52 | Tomato | FEMALE | 17.777 | 24.055 |
| | | MALE | 18.689 | 26.064 |
| 53 | Walnut_Blk | FEMALE | 45.379 | 56.909 |
| | | MALE | 45.121 | 56.368 |
| 54 | Wheat | FEMALE | 30.607 | 56.367 |
| | | MALE | 27.157 | 37.516 |
| 55 | Yeast_Baker | FEMALE | 9.254 | 12.440 |
| | | MALE | 15.276 | 36.374 |
| 56 | Yeast_Brewer | FEMALE | 20.592 | 26.569 |
| | | MALE | 40.875 | 97.645 |
| 57 | Peanut | FEMALE | 11.256 | 16.409 |
| | | MALE | 6.855 | 9.023 |
| 58 | Pineapple | FEMALE | 64.496 | 122.29 |
| | | MALE | 67.328 | 107.03 |
| 59 | Sole | FEMALE | 9.501 | 14.696 |
| | | MALE | 7.457 | 9.211 |
| 60 | Blueberry | FEMALE | 8.428 | 10.689 |
| | | MALE | 8.890 | 10.498 |
| 61 | Grape | FEMALE | 26.996 | 32.188 |
| | | MALE | 34.425 | 36.812 |
| 62 | Chicken | FEMALE | 32.645 | 39.638 |
| | | MALE | 31.388 | 38.932 |
| 63 | Cinnamon | FEMALE | 68.565 | 77.243 |
| | | MALE | 68.790 | 96.034 |
| 64 | Turkey | FEMALE | 25.025 | 29.329 |
| | | MALE | 27.468 | 34.845 |
| 65 | Butter | FEMALE | 47.272 | 70.707 |
| | | MALE | 44.283 | 58.138 |
| 66 | Cottage_Ch_{—} | FEMALE | 200.30 | 285.99 |
| | | MALE | 223.10 | 349.61 |
| 67 | Cashew | FEMALE | 23.342 | 45.186 |
| | | MALE | 17.535 | 32.327 |
| 68 | Yogurt | FEMALE | 45.514 | 63.745 |
| | | MALE | 43.700 | 66.542 |
| 69 | Cow_Milk | FEMALE | 198.53 | 247.06 |
| | | MALE | 184.55 | 316.82 |
| 70 | Egg | FEMALE | 142.74 | 281.40 |
| | | MALE | 106.90 | 198.06 |
| 71 | Millet | FEMALE | 7.808 | 17.593 |
| | | MALE | 5.898 | 7.419 |
| 72 | Coffee | FEMALE | 55.413 | 97.078 |
| | | MALE | 39.217 | 58.621 |
| 73 | Halibut | FEMALE | 17.373 | 25.326 |
| | | MALE | 21.523 | 31.890 |
| 74 | Beef | FEMALE | 16.869 | 27.375 |
| | | MALE | 16.113 | 29.309 |
| 75 | Swiss_Ch_{—} | FEMALE | 104.03 | 191.03 |
| | | MALE | 112.20 | 222.28 |
| 76 | Lobster | FEMALE | 23.224 | 29.796 |
| | | MALE | 29.842 | 39.104 |
| 77 | Parsley | FEMALE | 11.098 | 19.997 |
| | | MALE | 8.446 | 16.939 |
| 78 | Pork | FEMALE | 28.182 | 34.507 |
| | | MALE | 24.076 | 36.592 |
| 79 | Shrimp | FEMALE | 81.645 | 99.019 |
| | | MALE | 70.268 | 101.00 |
| 80 | Cheddar_Ch_{—} | FEMALE | 72.795 | 114.18 |
| | | MALE | 81.206 | 123.33 |
| 81 | Goat_Milk | FEMALE | 37.159 | 70.609 |
| | | MALE | 46.520 | 73.412 |
| 82 | Banana | FEMALE | 20.350 | 40.056 |
| | | MALE | 10.484 | 24.779 |
| 83 | Amer_Cheese | FEMALE | 54.269 | 90.667 |
| | | MALE | 56.316 | 96.580 |

TABLE 5A

| CROHN'S DISEASE POPULATION | | NON-CROHN'S DISEASE POPULATION | |
|---|---|---|---|
| | # of Positive | | # of Positive |
| | Results Based on | | Results Based on |
| Sample ID | 90th Percentile | Sample ID | 90th Percentile |
| 160905AAD0013 | 40 | BRH1244900 | 6 |
| 160905AAD0014 | 16 | BRH1244901 | 21 |
| 160905AAD0018 | 38 | BRH1244902 | 3 |
| 160905AAD0007 | 68 | BRH1244903 | 1 |
| 160905AAD0009 | 74 | BRH1244904 | 1 |
| BRH1281381 | 31 | BRH1244905 | 1 |
| BRH1281384 | 45 | BRH1244906 | 24 |
| BRH1281385 | 45 | BRH1244907 | 1 |
| BRH1281388 | 51 | BRH1244908 | 9 |
| BRH1281390 | 70 | BRH1244909 | 9 |
| BRH1281392 | 39 | BRH1244910 | 15 |
| BRH1281395 | 19 | BRH1244911 | 2 |
| BRH1281396 | 36 | BRH1244912 | 5 |
| BRH1274510 | 13 | BRH1244913 | 1 |
| BRH1274514 | 43 | BRH1244914 | 13 |
| BRH1274515 | 66 | BRH1244915 | 1 |
| BRH1274516 | 55 | BRH1244916 | 9 |
| BRH1274517 | 36 | BRH1244917 | 36 |
| BRH1274519 | 66 | BRH1244918 | 9 |
| BRH1274522 | 69 | BRH1244919 | 1 |
| BRH1274527 | 67 | BRH1244920 | 9 |
| BRH1274529 | 32 | BRH1244921 | 5 |
| BRH1274530 | 80 | BRH1244922 | 41 |
| BRH1274532 | 32 | BRH1244923 | 5 |
| BRH1274533 | 62 | BRH1244924 | 2 |
| BRH1282509 | 27 | BRH1244925 | 5 |
| BRH1282510 | 12 | BRH1244926 | 27 |
| BRH1282511 | 8 | BRH1244927 | 6 |
| BRH1282513 | 22 | BRH1244928 | 11 |
| BRH1282515 | 8 | BRH1244929 | 11 |
| BRH1282516 | 54 | BRH1244930 | 3 |
| BRH1282520 | 42 | BRH1244931 | 0 |
| BRH1282521 | 65 | BRH1244932 | 21 |
| BRH1282523 | 23 | BRH1244933 | 10 |
| BRH1282526 | 14 | BRH1244934 | 14 |
| BRH1282528 | 54 | BRH1244935 | 31 |
| BRH1282529 | 44 | BRH1244936 | 6 |
| KH16-18422 | 67 | BRH1244937 | 10 |
| KH16-18423 | 47 | BRH1244938 | 16 |
| KH16-18430 | 25 | BRH1244939 | 9 |
| KH16-19958 | 23 | BRH1244940 | 2 |
| KH16-20620 | 1 | BRH1244941 | 1 |
| 160905AAD0015 | 26 | BRH1244942 | 17 |
| 160905AAD0016 | 17 | BRH1244943 | 3 |
| 160905AAD0017 | 12 | BRH1244944 | 52 |
| 160905AAD0019 | 25 | BRH1244945 | 0 |
| 160905AAD0020 | 7 | BRH1244946 | 14 |
| 160905AAD0021 | 21 | BRH1244947 | 13 |
| 160905AAD0001 | 8 | BRH1244948 | 6 |
| 160905AAD0002 | 2 | BRH1244949 | 5 |
| 160905AAD0003 | 47 | BRH1244950 | 4 |
| 160905AAD0004 | 13 | BRH1244951 | 0 |
| 160905AAD0005 | 33 | BRH1244952 | 5 |
| 160905AAD0006 | 19 | BRH1244953 | 11 |
| 160905AAD0008 | 33 | BRH1244954 | 0 |
| 160905AAD0010 | 17 | BRH1244955 | 0 |
| 160905AAD0011 | 66 | BRH1244956 | 58 |
| 160905AAD0012 | 43 | BRH1244957 | 6 |
| BRH1281380 | 17 | BRH1244958 | 8 |
| BRH1281382 | 15 | BRH1244959 | 4 |
| BRH1281383 | 34 | BRH1244960 | 1 |
| BRH1281386 | 37 | BRH1244961 | 1 |
| BRH1281387 | 61 | BRH1244962 | 5 |
| BRH1281389 | 62 | BRH1244963 | 11 |
| BRH1281391 | 38 | BRH1244964 | 12 |
| BRH1281393 | 53 | BRH1244965 | 7 |
| BRH1281394 | 4 | BRH1244966 | 2 |
| BRH1281397 | 22 | BRH1244967 | 4 |
| BRH1281398 | 5 | BRH1244968 | 2 |
| BRH1281399 | 13 | BRH1244969 | 3 |
| BRH1281400 | 15 | BRH1244970 | 14 |
| BRH1281401 | 1 | BRH1244971 | 21 |
| BRH1274511 | 28 | BRH1244972 | 3 |
| BRH1274512 | 7 | BRH1244973 | 8 |
| BRH1274513 | 2 | BRH1244974 | 1 |
| BRH1274518 | 22 | BRH1244975 | 0 |
| BRH1274520 | 32 | BRH1244976 | 4 |
| BRH1274521 | 57 | BRH1244977 | 0 |
| BRH1274523 | 18 | BRH1244978 | 0 |
| BRH1274524 | 62 | BRH1244979 | 0 |
| BRH1274525 | 16 | BRH1244980 | 4 |
| BRH1274526 | 56 | BRH1244981 | 3 |
| BRH1274528 | 45 | BRH1244982 | 0 |
| BRH1274531 | 25 | BRH1244983 | 2 |
| BRH1274534 | 21 | BRH1244984 | 6 |
| BRH1282508 | 2 | BRH1244985 | 8 |
| BRH1282512 | 50 | BRH1244986 | 0 |
| BRH1282514 | 19 | BRH1244987 | 1 |
| BRH1282517 | 27 | BRH1244988 | 11 |
| BRH1282518 | 9 | BRH1244989 | 4 |
| BRH1282519 | 6 | BRH1244990 | 2 |
| BRH1282522 | 7 | BRH1244991 | 1 |
| BRH1282524 | 18 | BRH1244992 | 3 |
| BRH1282525 | 58 | BRH1267320 | 0 |
| BRH1282527 | 34 | BRH1267321 | 19 |
| BRH1282530 | 28 | BRH1267322 | 10 |
| BRH1282531 | 41 | BRH1267323 | 0 |
| KH16-18425 | 6 | BRH1244993 | 2 |
| KH16-19955 | 1 | BRH1244994 | 1 |
| KH16-19961 | 58 | BRH1244995 | 1 |
| No of Observations | 100 | BRH1244996 | 4 |
| Average Number | 32.5 | BRH1244997 | 4 |
| Median Number | 29.5 | BRH1244998 | 9 |
| # of Patients w/0 | 0 | BRH1244999 | 3 |
| Pos Results | | BRH1245000 | 10 |
| % Subjects w/0 | 0.0 | BRH1245001 | 4 |
| pos results | | BRH1245002 | 6 |
| | | BRH1245003 | 6 |
| | | BRH1245004 | 1 |
| | | BRH1245005 | 2 |
| | | BRH1245006 | 0 |
| | | BRH1245007 | 0 |
| | | BRH1245008 | 23 |
| | | BRH1245009 | 9 |
| | | BRH1245010 | 15 |
| | | BRH1245011 | 18 |
| | | BRH1245012 | 2 |
| | | BRH1245013 | 32 |
| | | BRH1245014 | 0 |
| | | BRH1245015 | 7 |
| | | BRH1245016 | 23 |
| | | BRH1245017 | 1 |
| | | BRH1245018 | 0 |
| | | BRH1245019 | 10 |
| | | BRH1245020 | 24 |
| | | BRH1245021 | 2 |
| | | BRH1245022 | 28 |
| | | BRH1245023 | 6 |
| | | BRH1245024 | 4 |
| | | BRH1245025 | 12 |
| | | BRH1245026 | 9 |
| | | BRH1245027 | 26 |
| | | BRH1245029 | 2 |
| | | BRH1245030 | 8 |
| | | BRH1245031 | 7 |
| | | BRH1245032 | 0 |
| | | BRH1245033 | 5 |
| | | BRH1245034 | 14 |
| | | BRH1245035 | 2 |
| | | BRH1245036 | 25 |
| | | BRH1245037 | 0 |
| | | BRH1245038 | 10 |
| | | BRH1245039 | 11 |
| | | BRH1245040 | 4 |
| | | BRH1245041 | 3 |
| | | BRH1267327 | 6 |
| | | BRH1267329 | 6 |
| | | BRH1267330 | 2 |
| | | BRH1267331 | 2 |
| | | BRH1267333 | 2 |
| | | BRH1267334 | 31 |
| | | BRH1267335 | 13 |
| | | BRH1267337 | 6 |
| | | BRH1267338 | 1 |
| | | BRH1267339 | 13 |
| | | BRH1267340 | 25 |
| | | BRH1267341 | 1 |
| | | BRH1267342 | 3 |
| | | BRH1267343 | 15 |
| | | BRH1267345 | 0 |
| | | BRH1267346 | 6 |
| | | BRH1267347 | 2 |
| | | BRH1267349 | 3 |
| | | No of Observations | 163 |
| | | Average Number | 8.1 |
| | | Median Number | 5 |
| | | # of Patients w/0 | 20 |
| | | Pos Results | |
| | | % Subjects w/0 | 12.3 |
| | | pos results | |

TABLE 5B

| CROHN'S DISEASE POPULATION | | NON-CROHN'S DISEASE POPULATION | |
|---|---|---|---|
| | # of Positive | | # of Positive |
| | Results Based on | | Results Based on |
| Sample ID | 95th Percentile | Sample ID | 95th Percentile |
| 160905AAD0013 | 26 | BRH1244900 | 2 |
| 160905AAD0014 | 13 | BRH1244901 | 9 |
| 160905AAD0018 | 12 | BRH1244902 | 3 |
| 160905AAD0007 | 57 | BRH1244903 | 0 |
| 160905AAD0009 | 65 | BRH1244904 | 1 |
| BRH1281381 | 9 | BRH1244905 | 0 |
| BRH1281384 | 32 | BRH1244906 | 10 |
| BRH1281385 | 30 | BRH1244907 | 1 |
| BRH1281388 | 27 | BRH1244908 | 4 |
| BRH1281390 | 67 | BRH1244909 | 6 |
| BRH1281392 | 23 | BRH1244910 | 7 |
| BRH1281395 | 12 | BRH1244911 | 1 |
| BRH1281396 | 22 | BRH1244912 | 2 |
| BRH1274510 | 3 | BRH1244913 | 0 |
| BRH1274514 | 27 | BRH1244914 | 7 |
| BRH1274515 | 54 | BRH1244915 | 0 |
| BRH1274516 | 44 | BRH1244916 | 5 |
| BRH1274517 | 21 | BRH1244917 | 21 |
| BRH1274519 | 62 | BRH1244918 | 1 |
| BRH1274522 | 58 | BRH1244919 | 1 |
| BRH1274527 | 57 | BRH1244920 | 5 |
| BRH1274529 | 20 | BRH1244921 | 2 |
| BRH1274530 | 80 | BRH1244922 | 21 |
| BRH1274532 | 25 | BRH1244923 | 3 |
| BRH1274533 | 51 | BRH1244924 | 2 |
| BRH1282509 | 21 | BRH1244925 | 1 |
| BRH1282510 | 4 | BRH1244926 | 20 |
| BRH1282511 | 1 | BRH1244927 | 3 |
| BRH1282513 | 9 | BRH1244928 | 3 |
| BRH1282515 | 4 | BRH1244929 | 7 |
| BRH1282516 | 42 | BRH1244930 | 1 |
| BRH1282520 | 25 | BRH1244931 | 0 |
| BRH1282521 | 51 | BRH1244932 | 8 |
| BRH1282523 | 9 | BRH1244933 | 3 |
| BRH1282526 | 10 | BRH1244934 | 5 |
| BRH1282528 | 34 | BRH1244935 | 17 |
| BRH1282529 | 30 | BRH1244936 | 3 |
| KH16-18422 | 55 | BRH1244937 | 3 |
| KH16-18423 | 28 | BRH1244938 | 5 |
| KH16-18430 | 16 | BRH1244939 | 2 |
| KH16-19958 | 13 | BRH1244940 | 1 |
| KH16-20620 | 0 | BRH1244941 | 1 |
| 160905AAD0015 | 18 | BRH1244942 | 11 |
| 160905AAD0016 | 11 | BRH1244943 | 2 |
| 160905AAD0017 | 7 | BRH1244944 | 19 |
| 160905AAD0019 | 17 | BRH1244945 | 0 |
| 160905AAD0020 | 6 | BRH1244946 | 7 |
| 160905AAD0021 | 10 | BRH1244947 | 4 |
| 160905AAD0001 | 1 | BRH1244948 | 0 |
| 160905AAD0002 | 1 | BRH1244949 | 3 |
| 160905AAD0003 | 31 | BRH1244950 | 1 |
| 160905AAD0004 | 10 | BRH1244951 | 0 |
| 160905AAD0005 | 16 | BRH1244952 | 2 |
| 160905AAD0006 | 11 | BRH1244953 | 3 |
| 160905AAD0008 | 22 | BRH1244954 | 0 |
| 160905AAD0010 | 8 | BRH1244955 | 0 |
| 160905AAD0011 | 55 | BRH1244956 | 43 |
| 160905AAD0012 | 24 | BRH1244957 | 4 |
| BRH1281380 | 10 | BRH1244958 | 1 |
| BRH1281382 | 10 | BRH1244959 | 1 |
| BRH1281383 | 20 | BRH1244960 | 0 |
| BRH1281386 | 26 | BRH1244961 | 1 |
| BRH1281387 | 45 | BRH1244962 | 2 |
| BRH1281389 | 58 | BRH1244963 | 3 |
| BRH1281391 | 24 | BRH1244964 | 5 |
| BRH1281393 | 43 | BRH1244965 | 3 |
| BRH1281394 | 0 | BRH1244966 | 1 |
| BRH1281397 | 12 | BRH1244967 | 1 |
| BRH1281398 | 1 | BRH1244968 | 1 |
| BRH1281399 | 6 | BRH1244969 | 1 |
| BRH1281400 | 11 | BRH1244970 | 3 |
| BRH1281401 | 1 | BRH1244971 | 10 |
| BRH1274511 | 16 | BRH1244972 | 2 |
| BRH1274512 | 1 | BRH1244973 | 4 |
| BRH1274513 | 2 | BRH1244974 | 1 |
| BRH1274518 | 13 | BRH1244975 | 0 |
| BRH1274520 | 20 | BRH1244976 | 2 |
| BRH1274521 | 51 | BRH1244977 | 0 |
| BRH1274523 | 8 | BRH1244978 | 0 |
| BRH1274524 | 43 | BRH1244979 | 0 |
| BRH1274525 | 8 | BRH1244980 | 2 |
| BRH1274526 | 44 | BRH1244981 | 2 |
| BRH1274528 | 29 | BRH1244982 | 0 |
| BRH1274531 | 10 | BRH1244983 | 2 |
| BRH1274534 | 16 | BRH1244984 | 2 |
| BRH1282508 | 1 | BRH1244985 | 3 |
| BRH1282512 | 32 | BRH1244986 | 0 |
| BRH1282514 | 6 | BRH1244987 | 0 |
| BRH1282517 | 23 | BRH1244988 | 8 |
| BRH1282518 | 6 | BRH1244989 | 1 |
| BRH1282519 | 1 | BRH1244990 | 1 |
| BRH1282522 | 4 | BRH1244991 | 1 |
| BRH1282524 | 14 | BRH1244992 | 1 |
| BRH1282525 | 49 | BRH1267320 | 0 |
| BRH1282527 | 21 | BRH1267321 | 15 |
| BRH1282530 | 15 | BRH1267322 | 3 |
| BRH1282531 | 30 | BRH1267323 | 0 |
| KH16-18425 | 3 | BRH1244993 | 0 |
| KH16-19955 | 1 | BRH1244994 | 0 |
| KH16-19961 | 47 | BRH1244995 | 1 |
| No of Observations | 100 | BRH1244996 | 2 |
| Average Number | 22.8 | BRH1244997 | 2 |
| Median Number | 17.5 | BRH1244998 | 5 |
| # of Patients w/0 | 2 | BRH1244999 | 2 |
| Pos Results | | BRH1245000 | 2 |
| % Subjects w/0 | 2.0 | BRH1245001 | 0 |
| pos results | | BRH1245002 | 1 |
| | | BRH1245003 | 2 |
| | | BRH1245004 | 0 |
| | | BRH1245005 | 1 |
| | | BRH1245006 | 0 |
| | | BRH1245007 | 0 |
| | | BRH1245008 | 16 |
| | | BRH1245009 | 5 |
| | | BRH1245010 | 5 |
| | | BRH1245011 | 9 |
| | | BRH1245012 | 0 |
| | | BRH1245013 | 9 |
| | | BRH1245014 | 0 |
| | | BRH1245015 | 2 |
| | | BRH1245016 | 7 |
| | | BRH1245017 | 0 |
| | | BRH1245018 | 0 |
| | | BRH1245019 | 8 |
| | | BRH1245020 | 14 |
| | | BRH1245021 | 0 |
| | | BRH1245022 | 15 |
| | | BRH1245023 | 2 |
| | | BRH1245024 | 1 |
| | | BRH1245025 | 7 |
| | | BRH1245026 | 6 |
| | | BRH1245027 | 15 |
| | | BRH1245029 | 0 |
| | | BRH1245030 | 4 |
| | | BRH1245031 | 4 |
| | | BRH1245032 | 0 |
| | | BRH1245033 | 1 |
| | | BRH1245034 | 8 |
| | | BRH1245035 | 0 |
| | | BRH1245036 | 9 |
| | | BRH1245037 | 0 |
| | | BRH1245038 | 9 |
| | | BRH1245039 | 5 |
| | | BRH1245040 | 0 |
| | | BRH1245041 | 0 |
| | | BRH1267327 | 4 |
| | | BRH1267329 | 3 |
| | | BRH1267330 | 2 |
| | | BRH1267331 | 1 |
| | | BRH1267333 | 1 |
| | | BRH1267334 | 15 |
| | | BRH1267335 | 7 |
| | | BRH1267337 | 4 |
| | | BRH1267338 | 0 |
| | | BRH1267339 | 6 |
| | | BRH1267340 | 20 |
| | | BRH1267341 | 1 |
| | | BRH1267342 | 1 |
| | | BRH1267343 | 12 |
| | | BRH1267345 | 0 |
| | | BRH1267346 | 3 |
| | | BRH1267347 | 1 |
| | | BRH1267349 | 2 |
| | | No of Observations | 163 |
| | | Average Number | 3.9 |
| | | Median Number | 2 |
| | | # of Patients w/0 | 39 |
| | | Pos Results | |
| | | % Subjects w/0 | 23.9 |
| | | pos results | |

TABLE 6A
Summary statistics

| | |
|---|---|
| Variable | Crohns_Disease_90th_percentile |
| | Crohns Disease 90th percentile |

| Sample size | 100 | |
|---|---|---|
| Lowest value | 1.0000 | |
| Highest value | 80.0000 | |
| Arithmetic mean | 32.5000 | |
| 95% Cl for the mean | 28.3134 to 36.6866 | |
| Median | 29.5000 | |
| 95% Cl for the median | 22.7234 to 36.2766 | |
| Variance | 445.1818 | |
| Standard deviation | 21.0993 | |
| Relative standard deviation | 0.6492 | (64.92%) |
| Standard error of the mean | 2.1099 | |
| Coefficient of Skewness | 0.3486 | (P = 0.1447) |
| Coefficient of Kurtosis | −0.9818 | (P = 0.0002) |

| | |
|---|---|
| D'Agostino-Pearson test | reject Normality |
| for Normal distribution | (P = 0.0004) |

| Percentiles | | 95% Confidence interval |
|---|---|---|
| 2.5 | 1.0000 | |
| 5 | 2.0000 | 1.0000 to 6.1396 |
| 10 | 6.5000 | 2.0000 to 8.7165 |
| 25 | 15.5000 | 9.8439 to 19.0000 |
| 75 | 48.5000 | 41.8038 to 57.7187 |
| 90 | 65.5000 | 58.0000 to 67.9461 |
| 95 | 67.5000 | 65.8604 to 75.5695 |
| 97.5 | 70.0000 | |

TABLE 6B
Summary statistics

| | |
|---|---|
| Variable | Crohns_Disease_95th_percentile |
| | Crohns Disease 95th percentile |

| Sample size | 100 | |
|---|---|---|
| Lowest value | 0.0000 | |
| Highest value | 80.0000 | |
| Arithmetic mean | 22.7800 | |
| 95% Cl for the mean | 18.9990 to 26.5610 | |
| Median | 17.5000 | |
| 95% Cl for the median | 12.7234 to 23.0000 | |
| Variance | 363.1026 | |
| Standard deviation | 19.0553 | |
| Relative standard deviation | 0.8365 | (83.65%) |
| Standard error of the mean | 1.9055 | |
| Coefficient of Skewness | 0.9010 | (P = 0.0006) |
| Coefficient of Kurtosis | −0.05980 | (P = 0.9588) |

| | |
|---|---|
| D'Agostino-Pearson test | reject Normality |
| for Normal distribution | (P = 0.0030) |

| Percentiles | | 95% Confidence interval |
|---|---|---|
| 2.6 | 1.0000 | |
| 5 | 1.0000 | 0.0000 to 1.0000 |
| 10 | 1.0000 | 1.0000 to 4.0000 |
| 25 | 8.5000 | 4.5626 to 10.1962 |
| 75 | 31.5000 | 26.0000 to 44.7187 |
| 90 | 54.5000 | 45.5670 to 58.0000 |
| 95 | 58.0000 | 54.8604 to 70.4006 |
| 97.5 | 65.0000 | |

TABLE 7A
Summary statistics

| | |
|---|---|
| Variable | Non_Crohns_Disease_90th_percentile |
| | Non-Crohns Disease 90th percentile |

| Sample size | 163 | |
|---|---|---|
| Lowest value | 0.0000 | |
| Highest value | 58.0000 | |
| Arithmetic mean | 8.1227 | |
| 95% Cl for the mean | 6.6171 to 9.6283 | |
| Median | 5.0000 | |
| 95% Cl for the median | 4.0000 to 6.0000 | |
| Variance | 94.7503 | |
| Standard deviation | 9.7340 | |
| Relative standard deviation | 1.1984 | (119.84%) |
| Standard error of the mean | 0.7624 | |
| Coefficient of Skewness | 2.2775 | (P < 0.0001) |
| Coefficient of Kurtosis | 6.6587 | (P < 0.0001) |

| | |
|---|---|
| D'Agostino-Pearson test | reject Normality |
| for Normal distribution | (P < 0.0001) |

| Percentiles | | 95% Confidence interval |
|---|---|---|
| 2.5 | 0.0000 | 0.0000 to 0.0000 |
| 5 | 0.0000 | 0.0000 to 0.0000 |
| 10 | 0.0000 | 0.0000 to 1.0000 |
| 25 | 2.0000 | 1.0000 to 2.0000 |
| 75 | 11.0000 | 9.0000 to 13.3243 |
| 90 | 21.4000 | 15.0000 to 26.2863 |
| 95 | 27.3500 | 23.5173 to 37.5705 |
| 97.5 | 33.7000 | 27.1327 to 56.7192 |

TABLE 7B
Summary statistics

| | |
|---|---|
| Variable | Non_Crohns_Disease_95th_percentile |
| | Non-Crohns Disease 95th percentile |

| Sample size | 163 | |
|---|---|---|
| Lowest value | 0.0000 | |
| Highest value | 43.0000 | |
| Arithmetic mean | 3.9325 | |
| 95% Cl for the mean | 3.0553 to 4.8097 | |
| Median | 2.0000 | |
| 95% Cl for the median | 1.0000 to 2.4934 | |
| Variance | 32.1621 | |
| Standard deviation | 5.6712 | |
| Relative standard deviation | 1.4421 | (144.21%) |
| Standard error of the mean | 0.4442 | |
| Coefficient of Skewness | 3.1127 | (P < 0.0001) |
| Coefficient of Kurtosis | 14.4768 | (P < 0.0001) |

| | |
|---|---|
| D'Agostino-Pearson test | reject Normality |
| for Normal distribution | (P < 0.0001) |

| Percentiles | | 95% Confidence interval |
|---|---|---|
| 2.5 | 0.0000 | 0.0000 to 0.0000 |
| 5 | 0.0000 | 0.0000 to 0.0000 |
| 10 | 0.0000 | 0.0000 to 0.0000 |
| 25 | 1.0000 | 0.0000 to 1.0000 |
| 75 | 5.0000 | 4.0000 to 7.0000 |
| 90 | 10.0000 | 8.0000 to 15.0000 |
| 95 | 15.3500 | 11.5173 to 20.3141 |
| 97.5 | 20.0000 | 15.1327 to 38.3037 |

TABLE 8A
Summary statistics

| | |
|---|---|
| Variable | Crohns_Disease_90th_percentile_1 |

Back-transformed after logarithmic transformation.

| Sample size | 100 | |
|---|---|---|
| Lowest value | 1.0000 | |
| Highest value | 80.1000 | |
| Geometric mean | 23.1743 | |
| 95% Cl for the mean | 18.9874 to 28.2845 | |
| Median | 29.4618 | |
| 95% Cl for the median | 22.7190 to 36.2738 | |
| Coefficient of Skewness | −1.3659 | (P < 0.0001) |
| Coefficient of Kurtosis | 1.7757 | (P = 0.0111) |

| | |
|---|---|
| D'Agostino-Pearson test | reject Normality |
| for Normal distribution | (P < 0.0001) |

| Percentiles | | 95% Confidence interval |
|---|---|---|
| 2.5 | 1.0000 | |
| 5 | 2.0000 | 1.0000 to 6.1305 |
| 10 | 6.4807 | 2.0000 to 8.7044 |
| 25 | 15.4919 | 9.7586 to 19.0000 |
| 75 | 48.5253 | 41.8018 to 57.7169 |
| 90 | 65.4981 | 58.0000 to 67.9458 |
| 95 | 67.4981 | 65.8595 to 75.5493 |
| 97.5 | 70.1000 | |

TABLE 8B
Summary statistics

| | |
|---|---|
| Variable | Crohns_Disease_95th_percentile_1 |

Back-transformed after logarithmic transformation.

| Sample size | 100 | |
|---|---|---|
| Lowest value | 0.1000 | |
| Highest value | 80.1000 | |
| Geometric mean | 13.1096 | |
| 95% Cl for the mean | 10.0330 to 17.1297 | |
| Median | 17.4929 | |
| 95% Cl for the median | 12.7154 to 23.0000 | |
| Coefficient of Skewness | −1.4090 | (P < 0.0001) |
| Coefficient of Kurtosis | 2.2772 | (P = 0.0035) |

| | |
|---|---|
| D'Agostino-Pearson test | reject Normality |
| for Normal distribution | (P < 0.0001) |

| Percentiles | | 95% Confidence interval |
|---|---|---|
| 2.5 | 1.0000 | |
| 5 | 1.0000 | 0.10000 to 1.0000 |
| 10 | 1.0000 | 1.0000 to 4.0000 |
| 25 | 8.4853 | 4.4833 to 10.2706 |
| 75 | 31.4960 | 26.0000 to 44.7164 |
| 90 | 54.4977 | 45.5582 to 58.0000 |
| 95 | 58.0000 | 54.8593 to 70.2042 |
| 97.5 | 65.0000 | |

TABLE 9A
Summary statistics

| | |
|---|---|
| Variable | Non_Crohns_Disease_90th_percentile_1 |
| | Non-Crohns Disease 90th percentile 1 |

Back-transformed after logarithmic transformation.

| Sample size | 163 | |
|---|---|---|
| Lowest value | 0.1000 | |
| Highest value | 58.0000 | |
| Geometric mean | 3.4215 | |
| 95% Cl for the mean | 2.6519 to 4.4146 | |
| Median | 5.0000 | |
| 95% Cl for the median | 4.0000 to 6.0000 | |
| Coefficient of Skewness | −0.8999 | (P < 0.0001) |
| Coefficient of Kurtosis | 0.1620 | (P = 0.5642) |

| | |
|---|---|
| D'Agostino-Pearson test | reject Normality |
| for Normal distribution | (P = 0.0001) |

| Percentiles | | 95% Confidence interval |
|---|---|---|
| 2.5 | 0.10000 | 0.10000 to 0.10000 |
| 5 | 0.10000 | 0.10000 to 0.10000 |
| 10 | 0.10000 | 0.10000 to 1.0000 |
| 25 | 2.0000 | 1.0000 to 2.0000 |
| 75 | 11.0000 | 9.0000 to 13.3162 |
| 90 | 21.3856 | 15.0000 to 26.2825 |
| 95 | 27.3459 | 23.5120 to 37.5010 |
| 97.5 | 33.6426 | 27.1306 to 56.6636 |

TABLE 9B
Summary statistics

| | |
|---|---|
| Variable | Non_Crohns_Disease_95th_percentile_1 |
| | Non-Crohns Disease 95th percentile_1 |

Back-transformed after logarithmic transformation.

| Sample size | 163 | |
|---|---|---|
| Lowest value | 0.1000 | |
| Highest value | 43.0000 | |
| Geometric mean | 1.4011 | |
| 95% Cl for the mean | 1.0770 to 1.8229 | |
| Median | 2.0000 | |
| 95% Cl for the median | 1.0000 to 2.4429 | |
| Coefficient of Skewness | −0.4141 | (P = 0.0313) |
| Coefficient of Kurtosis | −0.9300 | (P < 0.0001) |

| | |
|---|---|
| D'Agostino-Pearson test | reject Normality |
| for Normal distribution | (P < 0.0001) |

| Percentiles | | 95% Confidence interval |
|---|---|---|
| 2.5 | 0.10000 | 0.10000 to 0.10000 |
| 5 | 0.10000 | 0.10000 to 0.10000 |
| 10 | 0.10000 | 0.10000 to 0.10000 |
| 25 | 1.0000 | 0.10000 to 1.0000 |
| 75 | 5.0000 | 4.0000 to 7.0000 |
| 90 | 10.1000 | 8.0000 to 15.0000 |
| 95 | 15.3427 | 11.5065 to 20.3785 |
| 97.5 | 20.1000 | 15.1290 to 36.9001 |

TABLE 10A
Independent samples t-test
Sample 1

| | |
|---|---|
| Variable | Crohns_Disease_90th_percentile_1 |

Sample 2

| | |
|---|---|
| Variable | Non_Crohns_Disease_90th_percentile_1 |
| | Non-Crohns Disease 90th percentile_1 |

Back-transformed after logarithmic transformation.

| | Sample 1 | Sample 2 |
|---|---|---|
| Sample size | 100 | 163 |
| Geometric mean | 23.1743 | 3.4215 |
| 95% Cl for the mean | 18.9874 to 28.2845 | 2.6519 to 4.4146 |
| Variance of Logs | 0.1902 | 0.5119 |

| | |
|---|---|
| F-test for equal variances | P < 0.001 |

T-test (assuming equal variances )
Difference on Log-transformed scale

| | |
|---|---|
| Difference | −0.8308 |
| Standard Error | 0.07932 |
| 95% Cl of difference | −0.9870 to −0.6746 |
| Test statistic t | −10.474 |
| Degrees of Freedom (DF) | 261 |
| Two-tailed probability | P < 0.0001 |

Back-transformed results

| | |
|---|---|
| Ratio of geometric means | 0.1476 |
| 95% Cl of ratio | 0.1030 to 0.2115 |

TABLE 10B
Independent samples t-test
Sample 1

| | |
|---|---|
| Variable | Crohns_Disease_95th_percentile_1 |

Sample 2

| | |
|---|---|
| Variable | Non_Crohns_Disease_95th_percentile_1 |
| | Non-Crohns Disease 95th percentile_1 |

Back-transformed after logarithmic transformation.

| | Sample 1 | Sample 2 |
|---|---|---|
| Sample size | 100 | 163 |
| Geometric mean | 13.1096 | 1.4011 |
| 95% Cl for the mean | 10.0330 to 17.1297 | 1.0770 to 1.8229 |
| Variance of Logs | 0.3427 | 0.5459 |

| | |
|---|---|
| F-test for equal variances | P = 0.012 |

T-test (assuming equal variances)
Difference on Log-transformed scale

| | |
|---|---|
| Difference | −0.9711 |
| Standard Error | 0.08697 |
| 95% Cl of difference | −1.1424 to −0.7999 |
| Test statistic t | −11.166 |
| Degrees of Freedom (DF) | 261 |
| Two-tailed probability | P < 0.0001 |

Back-transformed results

| | |
|---|---|
| Ratio of geometric means | 0.1069 |
| 95% Cl of ratio | 0.07205 to 0.1585 |

TABLE 11A
Mann-Whitney test (independent samples)
Sample 1

| | |
|---|---|
| Variable | Crohns_Disease_90th_percentile |
| | Crohns Disease 90th percentile |

Sample 2

| | |
|---|---|
| Variable | Non_Crohns_Disease_90th_percentile |
| | Non-Crohns Disease 90th percentile |

| | Sample 1 | Sample 2 |
|---|---|---|
| Sample size | 100 | 163 |
| Lowest value | 1.0000 | 0.0000 |
| Highest value | 80.0000 | 58.0000 |
| Median | 29.5000 | 5.0000 |
| 95% Cl for the median | 22.7234 to 36.2766 | 4.0000 to 6.0000 |
| Interquartile range | 15.5000 to 48.5000 | 2.0000 to 11.0000 |

Mann-Whitney test (independent samples )

| | |
|---|---|
| Average rank of first group | 191.4500 |
| Average rank of second group | 95.5276 |
| Mann-Whitney U | 2205.00 |
| Test statistic Z (corrected for ties) | 9.936 |
| Two-tailed probability | P < 0.0001 |

TABLE 11B
Mann-Whitney test (independent samples)
Sample 1

| | |
|---|---|
| Variable | Crohns_Disease_95th_percentile |
| | Crohns Disease 95th percentile |

Sample 2

| | |
|---|---|
| Variable | Non_Crohns_Disease_95th_percentile |
| | Non-Crohns Disease 95th percentile |

| | Sample 1 | Sample 2 |
|---|---|---|
| Sample size | 100 | 163 |
| Lowest value | 0.0000 | 0.0000 |
| Highest value | 80.0000 | 43.0000 |
| Median | 17.5000 | 2.0000 |
| 95% Cl for the median | 12.7234 to 23.0000 | 1.0000 to 2.4934 |
| Interquartile range | 8.5000 to 31.5000 | 1.0000 to 5.0000 |

Mann -Whitney test (independent samples)

| | |
|---|---|
| Average rank of first group | 191.1850 |
| Average rank of second group | 95.6902 |
| Mann-Whitney U | 2231.50 |
| Test statistic Z (corrected for ties) | 9.924 |
| Two-tailed probability | P < 0.0001 |

TABLE 12A
ROC curve

| | |
|---|---|
| Variable | Crohns_Disease_Test_90th |
| | Crohns Disease Test_90th |
| Classification | Diagnosis_{——}1_Crohns_0_Non_Crohns_Disease_{—} |
| variable | Diagnosis(1_Crohns 0_Non-Crohns Disease) |

| | |
|---|---|
| Sample size | 263 |
| Positive group ^{a} | 100 (38.02%) |
| Negative group ^{b} | 163 (61.98%) |

| | |
|---|---|
| Disease prevalence (%) | unknown |

Area under the ROC curve (AUC)

| | |
|---|---|
| Area under the ROC curve (AUC) | 0.865 |
| Standard Error ^{a} | 0.0238 |
| 95% Confidence interval ^{b} | 0.817 to 0.904 |
| z statistic | 15.343 |
| Significance level P (Area = 0.5) | <0.0001 |

Youden index

| | |
|---|---|
| Youden index J | 0.6105 |
| 95% Confidence interval ² | 0.4833 to 0.6773 |
| Associated criterion | >14 |
| 95% Confidence interval ² | >11 to >21 |
| Sensitivity | 77.00 |
| Specificity | 84.05 |

^{a} Diagnosis_{——}1_Crohns_0_Non_Crohns_Disease_ = 1
^{b} Diagnosis_{——}1_Crohns_0_Non_Crohns_Disease_ = 0
^{a} DeLong et al.. 1988
^{b} Binomial exact
² BC₂ bootstrap confidence interval (1000 iterations; random number seed: 978).

TABLE 12B
ROC curve

| | |
|---|---|
| Variable | Crohns_Disease_Test_95th |
| | Crohns Disease Test_95th |
| Classification | Diagnosis_{——}1_Crohns_0_Non_Crohns_Disease_{—} |
| variable | Diagnosis(1_Crohns 0_Non-Crohns Disease) |

| | |
|---|---|
| Sample size | 263 |
| Positive group ^{a} | 100 (38.02%) |
| Negative group ^{b} | 163 (61.98%) |

| | |
|---|---|
| Disease prevalence (%) | unknown |

Area under the ROC curve (AUC)

| | |
|---|---|
| Area under the ROC curve (AUC) | 0.863 |
| Standard Error ^{a} | 0.0247 |
| 95% Confidence interval ^{b} | 0.816 to 0.902 |
| z statistic | 14.690 |
| Significance level P (Area = 0.5) | <0.0001 |

Youden index

| | |
|---|---|
| Youden index J | 0.6205 |
| 95% Confidence interval ² | 0.4976 to 0.6859 |
| Associated criterion | >7 |
| 95% Confidence interval ² | >5 to >9 |
| Sensitivity | 78.00 |
| Specificity | 84.05 |

^{a} Diagnosis_{——}1_Crohns_0_Non_Crohns_Disease_ = 1
^{b} Diagnosis_{——}1_Crohns_0_Non_Crohns_Disease_ = 0
^{a} DeLong et al., 1988
^{b} Binomial exact
² BC₂ bootstrap confidence interval (1000 iterations; random number seed: 978).

TABLE 13A
Performance Metrics in Predicting Crohn's Disease Status from Number of
Positive Foods Using 90th Percentile of ELISA Signal to determine Positive

| | No. of | | | | | |
|---|---|---|---|---|---|---|
| | Positive | | | Positive | Negative | Overall |
| | Foods as | | | Predictive | Predictive | Percent |
| Sex | Cutoff | Sensitivity | Specificity | Value | Value | Agreement |
| FEMALE | 1 | 1.00 | 0.05 | 0.48 | 1.00 | 0.49 |
| | 2 | 0.97 | 0.17 | 0.51 | 0.90 | 0.55 |
| | 3 | 0.95 | 0.28 | 0.54 | 0.86 | 0.59 |
| | 4 | 0.92 | 0.36 | 0.56 | 0.82 | 0.62 |
| | 5 | 0.90 | 0.42 | 0.58 | 0.82 | 0.64 |
| | 6 | 0.87 | 0.48 | 0.60 | 0.81 | 0.66 |
| | 7 | 0.84 | 0.53 | 0.61 | 0.79 | 0.68 |
| | 8 | 0.82 | 0.57 | 0.63 | 0.78 | 0.69 |
| | 9 | 0.80 | 0.60 | 0.64 | 0.77 | 0.70 |
| | 10 | 0.78 | 0.64 | 0.66 | 0.77 | 0.71 |
| | 11 | 0.77 | 0.68 | 0.68 | 0.77 | 0.72 |
| | 12 | 0.76 | 0.71 | 0.69 | 0.77 | 0.73 |
| | 13 | 0.74 | 0.73 | 0.71 | 0.76 | 0.73 |
| | 14 | 0.73 | 0.76 | 0.72 | 0.76 | 0.74 |
| | 15 | 0.71 | 0.78 | 0.74 | 0.76 | 0.74 |
| | 16 | 0.69 | 0.79 | 0.74 | 0.74 | 0.74 |
| | 17 | 0.67 | 0.81 | 0.75 | 0.73 | 0.74 |
| | 18 | 0.62 | 0.83 | 0.76 | 0.71 | 0.73 |
| | 19 | 0.59 | 0.84 | 0.76 | 0.70 | 0.72 |
| | 20 | 0.56 | 0.84 | 0.76 | 0.69 | 0.71 |
| | 21 | 0.54 | 0.85 | 0.76 | 0.68 | 0.70 |
| | 22 | 0.52 | 0.86 | 0.76 | 0.67 | 0.70 |
| | 23 | 0.50 | 0.87 | 0.77 | 0.67 | 0.70 |
| | 24 | 0.49 | 0.88 | 0.77 | 0.66 | 0.69 |
| | 25 | 0.47 | 0.89 | 0.79 | 0.66 | 0.69 |
| | 26 | 0.46 | 0.90 | 0.80 | 0.65 | 0.69 |
| | 27 | 0.45 | 0.91 | 0.81 | 0.65 | 0.69 |
| | 28 | 0.43 | 0.92 | 0.83 | 0.65 | 0.69 |
| | 29 | 0.41 | 0.93 | 0.84 | 0.64 | 0.69 |
| | 30 | 0.40 | 0.95 | 0.86 | 0.64 | 0.68 |
| | 31 | 0.38 | 0.95 | 0.87 | 0.63 | 0.68 |
| | 32 | 0.36 | 0.96 | 0.88 | 0.63 | 0.68 |
| | 33 | 0.34 | 0.97 | 0.90 | 0.63 | 0.68 |
| | 34 | 0.33 | 0.98 | 0.92 | 0.62 | 0.67 |
| | 35 | 0.31 | 0.98 | 0.93 | 0.62 | 0.67 |
| | 36 | 0.30 | 1.00 | 1.00 | 0.61 | 0.66 |
| | 37 | 0.28 | 1.00 | 1.00 | 0.61 | 0.66 |
| | 38 | 0.27 | 1.00 | 1.00 | 0.61 | 0.65 |
| | 39 | 0.26 | 1.00 | 1.00 | 0.61 | 0.65 |
| | 40 | 0.25 | 1.00 | 1.00 | 0.60 | 0.65 |
| | 41 | 0.24 | 1.00 | 1.00 | 0.60 | 0.64 |
| | 42 | 0.24 | 1.00 | 1.00 | 0.60 | 0.64 |
| | 43 | 0.23 | 1.00 | 1.00 | 0.59 | 0.64 |
| | 44 | 0.22 | 1.00 | 1.00 | 0.59 | 0.63 |
| | 45 | 0.21 | 1.00 | 1.00 | 0.59 | 0.63 |
| | 46 | 0.21 | 1.00 | 1.00 | 0.59 | 0.63 |
| | 47 | 0.20 | 1.00 | 1.00 | 0.59 | 0.63 |
| | 48 | 0.19 | 1.00 | 1.00 | 0.58 | 0.62 |
| | 49 | 0.18 | 1.00 | 1.00 | 0.58 | 0.62 |
| | 50 | 0.18 | 1.00 | 1.00 | 0.58 | 0.61 |
| | 51 | 0.17 | 1.00 | 1.00 | 0.58 | 0.61 |
| | 52 | 0.16 | 1.00 | 1.00 | 0.58 | 0.61 |
| | 53 | 0.15 | 1.00 | 1.00 | 0.57 | 0.61 |
| | 54 | 0.15 | 1.00 | 1.00 | 0.57 | 0.60 |
| | 55 | 0.14 | 1.00 | 1.00 | 0.57 | 0.60 |
| | 56 | 0.13 | 1.00 | 1.00 | 0.57 | 0.59 |
| | 57 | 0.12 | 1.00 | 1.00 | 0.56 | 0.59 |
| | 58 | 0.10 | 1.00 | 1.00 | 0.56 | 0.58 |
| | 59 | 0.08 | 1.00 | 1.00 | 0.55 | 0.57 |
| | 60 | 0.06 | 1.00 | 1.00 | 0.55 | 0.56 |
| | 61 | 0.05 | 1.00 | 1.00 | 0.55 | 0.56 |
| | 62 | 0.04 | 1.00 | 1.00 | 0.54 | 0.55 |
| | 63 | 0.03 | 1.00 | 1.00 | 0.54 | 0.55 |
| | 64 | 0.03 | 1.00 | 1.00 | 0.54 | 0.54 |
| | 65 | 0.03 | 1.00 | 1.00 | 0.54 | 0.54 |
| | 66 | 0.00 | 1.00 | 1.00 | 0.53 | 0.54 |
| | 67 | 0.00 | 1.00 | 1.00 | 0.53 | 0.54 |
| | 68 | 0.00 | 1.00 | 1.00 | 0.53 | 0.53 |
| | 69 | 0.00 | 1.00 | 1.00 | 0.53 | 0.53 |
| | 70 | 0.00 | 1.00 | 1.00 | 0.53 | 0.53 |
| | 71 | 0.00 | 1.00 | 1.00 | 0.53 | 0.53 |
| | 72 | 0.00 | 1.00 | 1.00 | 0.53 | 0.53 |
| | 73 | 0.00 | 1.00 | . | 0.53 | 0.53 |
| | 74 | 0.00 | 1.00 | . | 0.53 | 0.53 |
| | 75 | 0.00 | 1.00 | . | 0.53 | 0.53 |
| | 76 | 0.00 | 1.00 | . | 0.53 | 0.53 |
| | 77 | 0.00 | 1.00 | . | 0.53 | 0.53 |
| | 78 | 0.00 | 1.00 | . | 0.53 | 0.53 |
| | 79 | 0.00 | 1.00 | . | 0.53 | 0.53 |
| | 80 | 0.00 | 1.00 | . | 0.53 | 0.53 |
| | 81 | 0.00 | 1.00 | . | 0.53 | 0.53 |
| | 82 | 0.00 | 1.00 | . | 0.53 | 0.53 |
| | 83 | 0.00 | 1.00 | . | 0.53 | 0.53 |

TABLE 13B
Performance Metrics in Predicting Crohn's Disease Status from Number of
Positive Foods Using 90th Percentile of ELISA Signal to determine Positive

| | No. of | | | | | |
|---|---|---|---|---|---|---|
| | Positive | | | Positive | Negative | Overall |
| | Foods as | | | Predictive | Predictive | Percent |
| Sex | Cutoff | Sensitivity | Specificity | Value | Value | Agreement |
| MALE | 1 | 1.00 | 0.11 | 0.33 | 1.00 | 0.38 |
| | 2 | 0.97 | 0.20 | 0.35 | 0.94 | 0.44 |
| | 3 | 0.97 | 0.31 | 0.38 | 0.95 | 0.51 |
| | 4 | 0.97 | 0.38 | 0.41 | 0.96 | 0.56 |
| | 5 | 0.97 | 0.44 | 0.43 | 0.97 | 0.60 |
| | 6 | 0.97 | 0.49 | 0.45 | 0.97 | 0.64 |
| | 7 | 0.96 | 0.56 | 0.48 | 0.97 | 0.68 |
| | 8 | 0.96 | 0.60 | 0.51 | 0.97 | 0.70 |
| | 9 | 0.96 | 0.63 | 0.52 | 0.97 | 0.72 |
| | 10 | 0.93 | 0.66 | 0.54 | 0.96 | 0.74 |
| | 11 | 0.92 | 0.69 | 0.57 | 0.95 | 0.76 |
| | 12 | 0.91 | 0.73 | 0.59 | 0.95 | 0.78 |
| | 13 | 0.90 | 0.77 | 0.62 | 0.94 | 0.81 |
| | 14 | 0.89 | 0.79 | 0.66 | 0.94 | 0.82 |
| | 15 | 0.88 | 0.82 | 0.68 | 0.94 | 0.84 |
| | 16 | 0.86 | 0.84 | 0.69 | 0.93 | 0.84 |
| | 17 | 0.84 | 0.85 | 0.71 | 0.93 | 0.85 |
| | 18 | 0.83 | 0.86 | 0.72 | 0.92 | 0.85 |
| | 19 | 0.82 | 0.87 | 0.74 | 0.92 | 0.86 |
| | 20 | 0.81 | 0.89 | 0.75 | 0.92 | 0.86 |
| | 21 | 0.81 | 0.90 | 0.78 | 0.91 | 0.87 |
| | 22 | 0.79 | 0.91 | 0.79 | 0.91 | 0.87 |
| | 23 | 0.79 | 0.92 | 0.80 | 0.91 | 0.88 |
| | 24 | 0.78 | 0.92 | 0.81 | 0.90 | 0.88 |
| | 25 | 0.76 | 0.93 | 0.81 | 0.90 | 0.88 |
| | 26 | 0.75 | 0.93 | 0.82 | 0.90 | 0.88 |
| | 27 | 0.73 | 0.93 | 0.83 | 0.89 | 0.87 |
| | 28 | 0.72 | 0.94 | 0.83 | 0.89 | 0.87 |
| | 29 | 0.70 | 0.94 | 0.83 | 0.88 | 0.87 |
| | 30 | 0.69 | 0.94 | 0.84 | 0.88 | 0.87 |
| | 31 | 0.68 | 0.95 | 0.84 | 0.87 | 0.87 |
| | 32 | 0.67 | 0.95 | 0.85 | 0.87 | 0.86 |
| | 33 | 0.65 | 0.95 | 0.85 | 0.86 | 0.86 |
| | 34 | 0.63 | 0.95 | 0.86 | 0.86 | 0.86 |
| | 35 | 0.62 | 0.95 | 0.86 | 0.85 | 0.85 |
| | 36 | 0.60 | 0.95 | 0.86 | 0.85 | 0.85 |
| | 37 | 0.58 | 0.96 | 0.86 | 0.84 | 0.85 |
| | 38 | 0.56 | 0.97 | 0.87 | 0.84 | 0.84 |
| | 39 | 0.54 | 0.97 | 0.87 | 0.83 | 0.84 |
| | 40 | 0.52 | 0.97 | 0.88 | 0.83 | 0.84 |
| | 41 | 0.52 | 0.97 | 0.88 | 0.82 | 0.83 |
| | 42 | 0.50 | 0.97 | 0.88 | 0.82 | 0.83 |
| | 43 | 0.48 | 0.97 | 0.89 | 0.81 | 0.83 |
| | 44 | 0.46 | 0.98 | 0.90 | 0.81 | 0.82 |
| | 45 | 0.45 | 0.98 | 0.91 | 0.80 | 0.82 |
| | 46 | 0.43 | 0.98 | 0.90 | 0.80 | 0.82 |
| | 47 | 0.41 | 0.98 | 0.90 | 0.79 | 0.81 |
| | 48 | 0.40 | 0.98 | 0.90 | 0.79 | 0.81 |
| | 49 | 0.38 | 0.98 | 0.90 | 0.79 | 0.80 |
| | 50 | 0.37 | 0.98 | 0.90 | 0.78 | 0.80 |
| | 51 | 0.35 | 0.98 | 0.90 | 0.78 | 0.80 |
| | 52 | 0.33 | 0.98 | 0.90 | 0.78 | 0.79 |
| | 53 | 0.33 | 0.98 | 0.90 | 0.77 | 0.79 |
| | 54 | 0.32 | 0.98 | 0.90 | 0.77 | 0.78 |
| | 55 | 0.30 | 0.98 | 0.90 | 0.77 | 0.78 |
| | 56 | 0.29 | 0.98 | 0.89 | 0.76 | 0.78 |
| | 57 | 0.28 | 0.98 | 0.89 | 0.76 | 0.78 |
| | 58 | 0.28 | 0.98 | 0.90 | 0.76 | 0.77 |
| | 59 | 0.27 | 0.98 | 0.90 | 0.76 | 0.77 |
| | 60 | 0.27 | 1.00 | 1.00 | 0.76 | 0.77 |
| | 61 | 0.26 | 1.00 | 1.00 | 0.76 | 0.77 |
| | 62 | 0.26 | 1.00 | 1.00 | 0.76 | 0.77 |
| | 63 | 0.25 | 1.00 | 1.00 | 0.75 | 0.77 |
| | 64 | 0.24 | 1.00 | 1.00 | 0.75 | 0.77 |
| | 65 | 0.22 | 1.00 | 1.00 | 0.75 | 0.76 |
| | 66 | 0.20 | 1.00 | 1.00 | 0.74 | 0.76 |
| | 67 | 0.18 | 1.00 | 1.00 | 0.74 | 0.75 |
| | 68 | 0.15 | 1.00 | 1.00 | 0.73 | 0.74 |
| | 69 | 0.12 | 1.00 | 1.00 | 0.73 | 0.74 |
| | 70 | 0.09 | 1.00 | 1.00 | 0.72 | 0.73 |
| | 71 | 0.07 | 1.00 | 1.00 | 0.72 | 0.72 |
| | 72 | 0.07 | 1.00 | 1.00 | 0.71 | 0.72 |
| | 73 | 0.04 | 1.00 | 1.00 | 0.71 | 0.72 |
| | 74 | 0.04 | 1.00 | 1.00 | 0.71 | 0.71 |
| | 75 | 0.04 | 1.00 | 1.00 | 0.71 | 0.71 |
| | 76 | 0.04 | 1.00 | 1.00 | 0.71 | 0.71 |
| | 77 | 0.04 | 1.00 | 1.00 | 0.71 | 0.71 |
| | 78 | 0.03 | 1.00 | 1.00 | 0.70 | 0.71 |
| | 79 | 0.03 | 1.00 | 1.00 | 0.70 | 0.71 |
| | 80 | 0.03 | 1.00 | 1.00 | 0.70 | 0.71 |
| | 81 | 0.00 | 1.00 | 1.00 | 0.70 | 0.70 |
| | 82 | 0.00 | 1.00 | 1.00 | 0.70 | 0.70 |
| | 83 | 0.00 | 1.00 | . | 0.70 | 0.70 |

TABLE 14A
Performance Metrics in Predicting Crohn's Disease Status from Number of
Positive Foods Using 95th Percentile of ELISA Signal to determine Positive

| | No. of | | | | | |
|---|---|---|---|---|---|---|
| | Positive | | | Positive | Negative | Overall |
| | Foods as | | | Predictive | Predictive | Percent |
| Sex | Cutoff | Sensitivity | Specificity | Value | Value | Agreement |
| FEMALE | 1 | 1.00 | 0.17 | 0.51 | 1.00 | 0.55 |
| | 2 | 0.92 | 0.34 | 0.55 | 0.82 | 0.61 |
| | 3 | 0.87 | 0.46 | 0.58 | 0.80 | 0.65 |
| | 4 | 0.84 | 0.53 | 0.61 | 0.79 | 0.68 |
| | 5 | 0.81 | 0.60 | 0.64 | 0.78 | 0.69 |
| | 6 | 0.78 | 0.64 | 0.66 | 0.77 | 0.71 |
| | 7 | 0.76 | 0.69 | 0.68 | 0.76 | 0.72 |
| | 8 | 0.73 | 0.73 | 0.70 | 0.76 | 0.73 |
| | 9 | 0.70 | 0.77 | 0.73 | 0.74 | 0.74 |
| | 10 | 0.67 | 0.81 | 0.75 | 0.73 | 0.74 |
| | 11 | 0.63 | 0.83 | 0.76 | 0.72 | 0.73 |
| | 12 | 0.58 | 0.85 | 0.77 | 0.70 | 0.72 |
| | 13 | 0.55 | 0.86 | 0.78 | 0.69 | 0.71 |
| | 14 | 0.51 | 0.88 | 0.79 | 0.67 | 0.71 |
| | 15 | 0.50 | 0.89 | 0.79 | 0.67 | 0.70 |
| | 16 | 0.47 | 0.90 | 0.81 | 0.66 | 0.70 |
| | 17 | 0.45 | 0.91 | 0.82 | 0.66 | 0.70 |
| | 18 | 0.44 | 0.93 | 0.83 | 0.65 | 0.70 |
| | 19 | 0.42 | 0.93 | 0.86 | 0.65 | 0.69 |
| | 20 | 0.39 | 0.95 | 0.88 | 0.64 | 0.69 |
| | 21 | 0.38 | 0.96 | 0.90 | 0.64 | 0.68 |
| | 22 | 0.35 | 0.98 | 0.92 | 0.63 | 0.68 |
| | 23 | 0.33 | 0.98 | 0.94 | 0.63 | 0.68 |
| | 24 | 0.31 | 1.00 | 1.00 | 0.62 | 0.67 |
| | 25 | 0.29 | 1.00 | 1.00 | 0.62 | 0.67 |
| | 26 | 0.28 | 1.00 | 1.00 | 0.61 | 0.66 |
| | 27 | 0.26 | 1.00 | 1.00 | 0.61 | 0.65 |
| | 28 | 0.24 | 1.00 | 1.00 | 0.60 | 0.65 |
| | 29 | 0.24 | 1.00 | 1.00 | 0.60 | 0.65 |
| | 30 | 0.23 | 1.00 | 1.00 | 0.60 | 0.64 |
| | 31 | 0.22 | 1.00 | 1.00 | 0.59 | 0.64 |
| | 32 | 0.21 | 1.00 | 1.00 | 0.59 | 0.63 |
| | 33 | 0.20 | 1.00 | 1.00 | 0.59 | 0.63 |
| | 34 | 0.19 | 1.00 | 1.00 | 0.59 | 0.62 |
| | 35 | 0.18 | 1.00 | 1.00 | 0.58 | 0.62 |
| | 36 | 0.18 | 1.00 | 1.00 | 0.58 | 0.62 |
| | 37 | 0.17 | 1.00 | 1.00 | 0.58 | 0.61 |
| | 38 | 0.17 | 1.00 | 1.00 | 0.58 | 0.61 |
| | 39 | 0.16 | 1.00 | 1.00 | 0.58 | 0.61 |
| | 40 | 0.16 | 1.00 | 1.00 | 0.57 | 0.61 |
| | 41 | 0.15 | 1.00 | 1.00 | 0.57 | 0.60 |
| | 42 | 0.15 | 1.00 | 1.00 | 0.57 | 0.60 |
| | 43 | 0.14 | 1.00 | 1.00 | 0.57 | 0.60 |
| | 44 | 0.14 | 1.00 | 1.00 | 0.57 | 0.59 |
| | 45 | 0.13 | 1.00 | 1.00 | 0.57 | 0.59 |
| | 46 | 0.11 | 1.00 | 1.00 | 0.56 | 0.59 |
| | 47 | 0.11 | 1.00 | 1.00 | 0.56 | 0.58 |
| | 48 | 0.09 | 1.00 | 1.00 | 0.56 | 0.58 |
| | 49 | 0.08 | 1.00 | 1.00 | 0.55 | 0.57 |
| | 50 | 0.06 | 1.00 | 1.00 | 0.55 | 0.56 |
| | 51 | 0.05 | 1.00 | 1.00 | 0.55 | 0.56 |
| | 52 | 0.03 | 1.00 | 1.00 | 0.54 | 0.55 |
| | 53 | 0.03 | 1.00 | 1.00 | 0.54 | 0.55 |
| | 54 | 0.03 | 1.00 | 1.00 | 0.54 | 0.54 |
| | 55 | 0.02 | 1.00 | 1.00 | 0.54 | 0.54 |
| | 56 | 0.00 | 1.00 | 1.00 | 0.54 | 0.54 |
| | 57 | 0.00 | 1.00 | 1.00 | 0.53 | 0.54 |
| | 58 | 0.00 | 1.00 | 1.00 | 0.53 | 0.53 |
| | 59 | 0.00 | 1.00 | 1.00 | 0.53 | 0.53 |
| | 60 | 0.00 | 1.00 | 1.00 | 0.53 | 0.53 |
| | 61 | 0.00 | 1.00 | 1.00 | 0.53 | 0.53 |
| | 62 | 0.00 | 1.00 | 1.00 | 0.53 | 0.53 |
| | 63 | 0.00 | 1.00 | 1.00 | 0.53 | 0.53 |
| | 64 | 0.00 | 1.00 | 1.00 | 0.53 | 0.53 |
| | 65 | 0.00 | 1.00 | 1.00 | 0.53 | 0.53 |
| | 66 | 0.00 | 1.00 | . | 0.53 | 0.53 |
| | 67 | 0.00 | 1.00 | . | 0.53 | 0.53 |
| | 68 | 0.00 | 1.00 | . | 0.53 | 0.53 |
| | 69 | 0.00 | 1.00 | . | 0.53 | 0.53 |
| | 70 | 0.00 | 1.00 | . | 0.53 | 0.53 |
| | 71 | 0.00 | 1.00 | . | 0.53 | 0.53 |
| | 72 | 0.00 | 1.00 | . | 0.53 | 0.53 |
| | 73 | 0.00 | 1.00 | . | 0.53 | 0.53 |
| | 74 | 0.00 | 1.00 | . | 0.53 | 0.53 |
| | 75 | 0.00 | 1.00 | . | 0.53 | 0.53 |
| | 76 | 0.00 | 1.00 | . | 0.53 | 0.53 |
| | 77 | 0.00 | 1.00 | . | 0.53 | 0.53 |
| | 78 | 0.00 | 1.00 | . | 0.53 | 0.53 |
| | 79 | 0.00 | 1.00 | . | 0.53 | 0.53 |
| | 80 | 0.00 | 1.00 | . | 0.53 | 0.53 |
| | 81 | 0.00 | 1.00 | . | 0.53 | 0.53 |
| | 82 | 0.00 | 1.00 | . | 0.53 | 0.53 |
| | 83 | 0.00 | 1.00 | . | 0.53 | 0.53 |

TABLE 14B
Performance Metrics in Predicting Crohn's Disease Status from Number of
Positive Foods Using 95th Percentile of ELISA Signal to determine Positive

| | No. of | | | | | |
|---|---|---|---|---|---|---|
| | Positive | | | Positive | Negative | Overall |
| | Foods as | | | Predictive | Predictive | Percent |
| Sex | Cutoff | Sensitivity | Specificity | Value | Value | Agreement |
| MALE | 1 | 0.97 | 0.20 | 0.35 | 0.95 | 0.43 |
| | 2 | 0.96 | 0.36 | 0.40 | 0.96 | 0.55 |
| | 3 | 0.96 | 0.50 | 0.46 | 0.97 | 0.64 |
| | 4 | 0.96 | 0.61 | 0.52 | 0.97 | 0.71 |
| | 5 | 0.92 | 0.68 | 0.56 | 0.95 | 0.75 |
| | 6 | 0.90 | 0.73 | 0.60 | 0.94 | 0.79 |
| | 7 | 0.89 | 0.78 | 0.63 | 0.94 | 0.81 |
| | 8 | 0.88 | 0.81 | 0.67 | 0.94 | 0.83 |
| | 9 | 0.87 | 0.84 | 0.71 | 0.94 | 0.85 |
| | 10 | 0.86 | 0.86 | 0.73 | 0.93 | 0.86 |
| | 11 | 0.84 | 0.88 | 0.75 | 0.93 | 0.87 |
| | 12 | 0.82 | 0.89 | 0.77 | 0.92 | 0.87 |
| | 13 | 0.79 | 0.91 | 0.78 | 0.91 | 0.87 |
| | 14 | 0.75 | 0.92 | 0.79 | 0.90 | 0.87 |
| | 15 | 0.73 | 0.92 | 0.80 | 0.89 | 0.86 |
| | 16 | 0.71 | 0.93 | 0.81 | 0.88 | 0.86 |
| | 17 | 0.70 | 0.94 | 0.82 | 0.88 | 0.86 |
| | 18 | 0.68 | 0.94 | 0.83 | 0.87 | 0.86 |
| | 19 | 0.67 | 0.95 | 0.83 | 0.87 | 0.86 |
| | 20 | 0.65 | 0.95 | 0.84 | 0.86 | 0.86 |
| | 21 | 0.64 | 0.95 | 0.85 | 0.86 | 0.85 |
| | 22 | 0.62 | 0.95 | 0.86 | 0.85 | 0.85 |
| | 23 | 0.59 | 0.96 | 0.87 | 0.85 | 0.85 |
| | 24 | 0.57 | 0.97 | 0.88 | 0.84 | 0.84 |
| | 25 | 0.54 | 0.97 | 0.89 | 0.83 | 0.84 |
| | 26 | 0.52 | 0.97 | 0.89 | 0.82 | 0.83 |
| | 27 | 0.49 | 0.98 | 0.90 | 0.82 | 0.83 |
| | 28 | 0.47 | 0.98 | 0.91 | 0.81 | 0.82 |
| | 29 | 0.44 | 0.98 | 0.91 | 0.80 | 0.82 |
| | 30 | 0.42 | 0.98 | 0.92 | 0.80 | 0.82 |
| | 31 | 0.40 | 0.98 | 0.92 | 0.79 | 0.81 |
| | 32 | 0.38 | 0.98 | 0.91 | 0.79 | 0.81 |
| | 33 | 0.37 | 0.98 | 0.91 | 0.79 | 0.80 |
| | 34 | 0.36 | 0.98 | 0.91 | 0.78 | 0.80 |
| | 35 | 0.34 | 0.98 | 0.91 | 0.78 | 0.80 |
| | 36 | 0.33 | 0.98 | 0.91 | 0.78 | 0.79 |
| | 37 | 0.33 | 0.98 | 0.91 | 0.77 | 0.79 |
| | 38 | 0.32 | 0.98 | 0.91 | 0.77 | 0.79 |
| | 39 | 0.32 | 0.98 | 0.91 | 0.77 | 0.79 |
| | 40 | 0.31 | 0.98 | 0.91 | 0.77 | 0.79 |
| | 41 | 0.31 | 0.98 | 0.91 | 0.77 | 0.78 |
| | 42 | 0.30 | 0.98 | 0.91 | 0.77 | 0.78 |
| | 43 | 0.30 | 0.99 | 0.91 | 0.77 | 0.78 |
| | 44 | 0.29 | 1.00 | 1.00 | 0.76 | 0.78 |
| | 45 | 0.29 | 1.00 | 1.00 | 0.76 | 0.78 |
| | 46 | 0.28 | 1.00 | 1.00 | 0.76 | 0.78 |
| | 47 | 0.27 | 1.00 | 1.00 | 0.76 | 0.78 |
| | 48 | 0.26 | 1.00 | 1.00 | 0.76 | 0.77 |
| | 49 | 0.25 | 1.00 | 1.00 | 0.76 | 0.77 |
| | 50 | 0.24 | 1.00 | 1.00 | 0.75 | 0.77 |
| | 51 | 0.23 | 1.00 | 1.00 | 0.75 | 0.77 |
| | 52 | 0.22 | 1.00 | 1.00 | 0.75 | 0.76 |
| | 53 | 0.21 | 1.00 | 1.00 | 0.75 | 0.76 |
| | 54 | 0.21 | 1.00 | 1.00 | 0.74 | 0.76 |
| | 55 | 0.19 | 1.00 | 1.00 | 0.74 | 0.76 |
| | 56 | 0.18 | 1.00 | 1.00 | 0.74 | 0.75 |
| | 57 | 0.16 | 1.00 | 1.00 | 0.73 | 0.75 |
| | 58 | 0.14 | 1.00 | 1.00 | 0.73 | 0.74 |
| | 59 | 0.13 | 1.00 | 1.00 | 0.73 | 0.74 |
| | 60 | 0.12 | 1.00 | 1.00 | 0.73 | 0.74 |
| | 61 | 0.11 | 1.00 | 1.00 | 0.72 | 0.73 |
| | 62 | 0.10 | 1.00 | 1.00 | 0.72 | 0.73 |
| | 63 | 0.08 | 1.00 | 1.00 | 0.72 | 0.73 |
| | 64 | 0.07 | 1.00 | 1.00 | 0.71 | 0.72 |
| | 65 | 0.07 | 1.00 | 1.00 | 0.71 | 0.72 |
| | 66 | 0.04 | 1.00 | 1.00 | 0.71 | 0.71 |
| | 67 | 0.04 | 1.00 | 1.00 | 0.71 | 0.71 |
| | 68 | 0.04 | 1.00 | 1.00 | 0.71 | 0.71 |
| | 69 | 0.04 | 1.00 | 1.00 | 0.71 | 0.71 |
| | 70 | 0.03 | 1.00 | 1.00 | 0.70 | 0.71 |
| | 71 | 0.03 | 1.00 | 1.00 | 0.70 | 0.71 |
| | 72 | 0.03 | 1.00 | 1.00 | 0.70 | 0.71 |
| | 73 | 0.03 | 1.00 | 1.00 | 0.70 | 0.71 |
| | 74 | 0.03 | 1.00 | 1.00 | 0.70 | 0.71 |
| | 75 | 0.03 | 1.00 | 1.00 | 0.70 | 0.71 |
| | 76 | 0.03 | 1.00 | 1.00 | 0.70 | 0.70 |
| | 77 | 0.00 | 1.00 | 1.00 | 0.70 | 0.70 |
| | 78 | 0.00 | 1.00 | 1.00 | 0.70 | 0.70 |
| | 79 | 0.00 | 1.00 | 1.00 | 0.70 | 0.70 |
| | 80 | 0.00 | 1.00 | 1.00 | 0.70 | 0.70 |
| | 81 | 0.00 | 1.00 | 1.00 | 0.70 | 0.70 |
| | 82 | 0.00 | 1.00 | . | 0.70 | 0.70 |
| | 83 | 0.00 | 1.00 | . | 0.70 | 0.70 |

1.-100. (canceled)

101. A method of identifying one or more trigger foods that when consumed by a subject diagnosed with or suspected to have Crohn's Disease, then causes or exacerbates the symptoms of Crohn's Disease, comprising:
obtaining test results for a plurality of distinct food preparations, wherein the test results are derived from a process that includes contacting the plurality of distinct food preparations with bodily fluids from patients diagnosed with or suspected of having Crohn's Disease, and bodily fluids from a control group not diagnosed with or not suspected of having Crohn's Disease;
identifying a plurality of distinct Crohn's Disease trigger food preparations, wherein a Crohn's Disease trigger food preparation exhibits a raw p-value of ≤0.07 or an FDR multiplicity adjusted p-value of ≤0.10 with respect to triggering symptoms of Crohn's Disease;
contacting a plurality of the distinct Crohn's Disease trigger food preparations with serum of a subject that is diagnosed with or suspected to have Crohn's Disease, wherein the step of contacting is performed under conditions that allow IgG from the serum to bind to food antigens of each of the plurality of distinct food preparations;
measuring IgG bound to the food antigens of each of the plurality of distinct Crohn's Disease trigger food preparations to obtain a signal for each of the plurality of distinct Crohn's Disease trigger food preparations;
comparing the signal obtained for each of the plurality of distinct Crohn's Disease trigger food preparations to a reference value for the distinct Crohn's Disease trigger food preparation; and
identifying one or more of the plurality of distinct Crohn's Disease trigger foods for the subject known to have or suspected of having Crohn's Disease based on the comparison of the signal to the reference signal for each of the plurality of distinct Crohn's Disease trigger food preparations.

102. The method of claim 101, wherein the reference value of each of the plurality of distinct Crohn's Disease trigger food preparation is set as the 90th percentile rank, or higher, of signals obtained by contacting serum from a control group of subjects that is not diagnosed with or suspected of having Crohn's Disease with each of the distinct Crohn's Disease trigger food preparations, and wherein a Crohn's Disease trigger food preparation is identified if the signal for the distinct Crohn's Disease trigger food preparation is larger than the reference value.

103. The method of claim 101, wherein the test result is an ELISA test result.

104. The method of claim 101, wherein the Crohn's Disease trigger food preparation exhibits a raw p-value of ≤0.05 or an FDR multiplicity adjusted p-value of ≤0.08 with respect to triggering symptoms of Crohn's Disease.

105. The method of claim 101, wherein the Crohn's Disease trigger food preparation exhibits a raw p-value of ≤0.025 or an FDR multiplicity adjusted p-value of ≤0.07 with respect to triggering symptoms of Crohn's Disease.

106. The method of claim 101, wherein the bodily fluid of the patient is whole blood, plasma, serum, saliva, or a fecal suspension.

107. The method of claim 101, further comprising a step of normalizing the measured IgG to the patient's total IgG.

108. The method of claim 101, further comprising a step of normalizing the measured IgG to a global mean of the patient's food specific IgG results.

109. A method of for identifying one or more Crohn's Disease trigger foods for a subject diagnosed with or suspected to have Crohn's Disease, comprising:
contacting a plurality of distinct Crohn's Disease trigger food preparations with serum of a subject that is diagnosed with or suspected to have Crohn's Disease, wherein the Crohn's Disease trigger food preparations are food preparations selected from the group consisting of almond, apple, avocado, barley, broccoli, buckwheat, cabbage, sugar cane, cantaloupe, carrot, cauliflower, celery, chili pepper, chocolate, clam, cola nut, corn, cucumber, eggplant, garlic, grapefruit, green pea, green pepper, honey, lemon, lettuce, lima bean, malt, mustard, oat, olive, onion, orange, oyster, peach, pinto bean, potato, rice, rye, safflower, sardine, scallop, soybean, spinach, squashes, strawberry, string bean, sunflower seed, sweet potato, tea, tobacco, tomato, walnut, wheat, baker's yeast, brewer's yeast, peanut, pineapple, sole, blueberry, grape, chicken, cinnamon, turkey, butter, cottage cheese, cashew, yogurt, cow's milk, egg, millet, coffee, halibut, beef, Swiss cheese, lobster, parsley, pork, shrimp, cheddar cheese, goat's milk, banana, and American cheese, wherein the step of contacting is performed under conditions that allow IgG from the serum to bind to food antigens of each of the plurality of distinct food preparations;
measuring IgG bound to the food antigens of each of the plurality of distinct Crohn's Disease trigger food preparations to obtain a signal for each of the plurality of distinct Crohn's Disease trigger food preparations;
comparing the signal obtained for each of the plurality of distinct Crohn's Disease trigger food preparations to a reference value for the distinct Crohn's Disease trigger food preparation; and
identifying one or more of the plurality of distinct Crohn's Disease trigger foods for the subject known to have or suspected of having Crohn's Disease based on the comparison of the signal to the reference signal for each of the plurality of distinct Crohn's Disease trigger food preparations.

110. The method of claim 109, wherein the reference value of each of the plurality of distinct Crohn's Disease trigger food preparation is set as the 90th percentile rank, or higher, of signals obtained by contacting serum from a control group of subjects that is not diagnosed with or suspected of having Crohn's Disease with each of the distinct Crohn's Disease trigger food preparations, and wherein a Crohn's Disease trigger food preparation is identified if the signal for the distinct Crohn's Disease trigger food preparation is larger than the reference value.

111. The method of claim 109, wherein the Crohn's Disease trigger food preparations are food preparations selected from the group consisting of almond, apple, avocado, barley, broccoli, buckwheat, cabbage, sugar cane, cantaloupe, carrot, cauliflower, celery, chili pepper, chocolate, clam, cola nut, corn, cucumber, eggplant, garlic, grapefruit, green pea, green pepper, honey, lemon, lettuce, lima bean, malt, mustard, oat, olive, onion, orange, oyster, peach, pinto bean, potato, rice, rye, safflower, sardine, scallop, soybean, spinach, squashes, strawberry, string bean, sunflower seed, sweet potato, tea, tobacco, tomato, walnut, wheat, baker's yeast, brewer's yeast, peanut, pineapple, sole, blueberry, grape, chicken, cinnamon, turkey, butter, cottage cheese, cashew, yogurt, cow's milk, egg, millet, coffee, halibut, beef, Swiss cheese, lobster, parsley, pork, shrimp, and cheddar cheese.

112. The method of claim 109, wherein the Crohn's Disease trigger food preparations are food preparations selected from the group consisting of almond, apple, avocado, barley, broccoli, buckwheat, cabbage, sugar cane, cantaloupe, carrot, cauliflower, celery, chili pepper, chocolate, clam, cola nut, corn, cucumber, eggplant, garlic, grapefruit, green pea, green pepper, honey, lemon, lettuce, lima bean, malt, mustard, oat, olive, onion, orange, oyster, peach, pinto bean, potato, rice, rye, safflower, sardine, scallop, soybean, spinach, squashes, strawberry, string bean, sunflower seed, sweet potato, tea, tobacco, tomato, walnut, wheat, baker's yeast, brewer's yeast, peanut, pineapple, sole, blueberry, grape, chicken, cinnamon, turkey, butter, cottage cheese, cashew, yogurt, cow's milk, egg, millet, coffee, halibut, beef, Swiss cheese, lobster, and parsley.

113. The method of claim 109, wherein the Crohn's Disease trigger food preparations are food preparations selected from the group consisting of almond, apple, avocado, barley, broccoli, buckwheat, cabbage, sugar cane, cantaloupe, carrot, cauliflower, celery, chili pepper, chocolate, clam, cola nut, corn, cucumber, eggplant, garlic, grapefruit, green pea, green pepper, honey, lemon, lettuce, lima bean, malt, mustard, oat, olive, onion, orange, oyster, peach, pinto bean, potato, rice, rye, safflower, sardine, scallop, soybean, spinach, squashes, strawberry, string bean, sunflower seed, sweet potato, tea, tobacco, tomato, walnut, wheat, baker's yeast, brewer's yeast, peanut, pineapple, sole, blueberry, grape, chicken, cinnamon, turkey, butter, cottage cheese, cashew, yogurt, cow's milk, egg, millet, coffee, and halibut.

114. The method of claim 109, further comprising a step of normalizing the measured IgG to the patient's total IgG.

115. The method of claim 109, further comprising a step of normalizing the measured IgG to a global mean of the patient's food specific IgG results.
